(19) United States
(12) Patent Application Publication
(10) Pub. No.: US 2013/0178453 A1
(43) Pub. Date: Jul. 11, 2013

(54) Cannabinoid Agonists

(71) Applicants: Jason Rohde, Rockville, MD (US); Bo Peng, Arlington, MA (US); Takashi Nakai, Newton, MA (US); Kevin Sprott, Nedham, MA (US); Ara Mermerían, Melrose, MA (US); Wayne C. Schairer, Westboro, MA (US); Galen John Carey, Stow, MA (US)

(72) Inventors: Jason Rohde, Rockville, MD (US); Bo Peng, Arlington, MA (US); Takashi Nakai, Newton, MA (US); Kevin Sprott, Nedham, MA (US); Ara Mermerían, Melrose, MA (US); Wayne C. Schairer, Westboro, MA (US); Galen John Carey, Stow, MA (US)

(73) Assignee: Ironwood Pharmaceuticals, Inc., Cambridge, MA (US)

(21) Appl. No.: 13/578,009

(22) PCT Filed: Feb. 9, 2011
(86) PCT No.: PCT/US2011/024237

§ 371 Date: Mar. 22, 2013

Related U.S. Application Data
(60) Provisional application No. 61/302,746, filed on Feb. 9, 2010.

(51) Int. Cl. C07D 495/04; C07D 491/048; A61K 31/5377; A61K 31/437; A61K 31/4709; A61K 31/429; C07D 513/04; A61K 45/06; A61K 31/407; A61K 31/404; A61K 31/424

(52) U.S. Cl. CPC C07D 495/04; A61K 31/404; C07D 491/048; A61K 31/5377; A61K 31/437; A61K 31/424; A61K 31/429; C07D 513/04; A61K 45/06; A61K 31/407; A61K 31/4709
USPC 514/171; 548/454; 514/414; 548/453; 544/143; 514/233.8; 546/113; 514/300; 514/412; 548/231; 514/392; 514/367; 548/153; 514/221; 546/168; 514/314; 435/375

(57) ABSTRACT

The present disclosure relates to compounds useful as agonists of the cannabinoid receptors. The disclosure also provides pharmaceutically acceptable compositions comprising the compounds of the disclosure and methods of using the compositions in the treatment of various disorders, either alone or in combination therapy. The compounds of the invention have Formula (I).

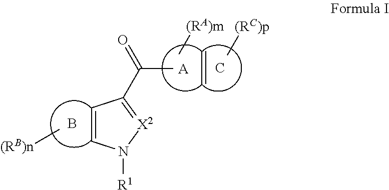

### PRIORITY CLAIM

This application claims priority to U.S. Provisional Application No. 61/302,746, filed on Feb. 9, 2010. The entire contents of the aforementioned application are incorporated herein by reference.

### TECHNICAL FIELD

The present disclosure relates to compounds useful as agonists of the cannabinoid receptors (CB receptors). The disclosure also provides pharmaceutically acceptable compositions comprising the compounds of the disclosure and methods of using the compositions in the treatment of various disorders either alone or in combination therapy.

### BACKGROUND

Cannabinoids are a group of compounds found in Cannabis sativa (also known as marijuana). Cannabis has been used in the treatment of various illnesses ranging from lack of appetite, emesis, cramps, menstrual pain, cancer pain and spasticity. Despite its clinical benefits, the therapeutic usage of cannabis is limited by its psychoactive effects including hallucination, addiction and dependence.

The physiological effects of cannabinoids are mediated by at least two G-protein coupled receptors, cannabinoid receptors 1 and 2 (CB1 and CB2). CB 1 receptors are expressed primarily in the central nervous system and are thought to mediate many of the psychoactive effects of cannabis. CB2 receptors are predominantly found in the immune system. CB2 receptors are expressed on inflammatory cells (T cells, B cells, macrophages, mast cells) and mediate immune suppression through inhibition of cellular interaction/inflammatory mediator release. More recent data also suggests a role for CB2 receptor activation in the central nervous system (CNS). CB2 receptor expression appears to be induced by inflammatory pain in a rat spinal cord model, which coincided with the appearance of activated microglia. CB2 receptor agonists also have been shown to reduce mechanically evoked responses and wind-up of wide dynamic range neurons in spinal cord dorsal horn in animal models of inflammatory pain. CB2 up-regulation was also observed in lesioned areas of brains in an animal model mimicking Alzheimer's disease. In addition, hepatic expression of CB2 receptors has also been observed in patients with chronic liver disease, and activation of CB2 receptors can trigger potent growth inhibitory and apoptotic effects, two major antifibrogenic properties, in hepatic myofibroblasts.

Cannabinoid receptor (CB) agonists may be used for treating immune disorders, liver fibrosis, inflammation and disorders that have an inflammatory component, such as cardiovascular disease, osteoporosis and renal ischemia. In addition, CB agonists may also be used in the treatment of emesis and pain including acute, chronic, inflammatory, post-operative, cancer and neuropathic pain.

### SUMMARY

The compounds disclosed herein, and pharmaceutically acceptable compositions thereof, are effective as agonists of the cannabinoid receptors. These compounds have the general Formula I, or are pharmaceutically acceptable salts thereof:

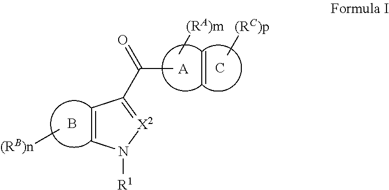

wherein
R¹ is -V-R⁸;
V is a single bond or a divalent linker between R⁸ and the nitrogen to which V is bonded, wherein said linker is a C₁₋₆ aliphatic; and wherein up to two saturated carbons of said C₁₋₆ aliphatic are optionally and independently replaced by —O—, —C(O)—, —C(S)—, —C(O)N(R)—, —N(R)C(O)—, —(R)NC(O)N(R)—, —C(O)O—, —OC(O)—, —N(R)—, —N(R)S(O)₂—, —S(O)₂N(R)—, N(R)S(O)₂N(R)— or —S(O)_{q}—;
each q is an integer independently selected from 0, 1 or 2;
each occurrence of R is independently selected from hydrogen, a C₁₋₄ alkyl, C₁₋₄ haloalkyl, C₃₋₆ cycloalkyl or —C(O)(C₁₋₄ alkyl);
R⁸ is selected from hydrogen, halogen, —CN, —NO₂, phenyl, a 5-6-membered heteroaryl ring, a C₃₋₁₀ cycloaliphatic ring or a 3-10-membered heterocyclyl ring; wherein each said phenyl, heteroaryl, cycloaliphatic or heterocyclyl ring is optionally and independently substituted with up to 6 instances of R¹⁵;
each occurrence of R¹⁵ is independently selected from halogen, oxo, —NO₂, —CN, —OR¹⁶, —N(R¹⁶)₂, —C(O)OR¹⁶, —C(O)R¹⁶, —N(R′)C(O)R¹⁶, —C(O)N(R¹⁶)₂, —OC(O)R¹⁶, —SR¹⁶, —S(O)₂R¹⁶, —SO₂N(R¹⁶)₂, —S(O)R¹⁶, a C₁₋₆ aliphatic or a C₃₋₆ cycloaliphatic; wherein each of said C₁₋₆ aliphatic and C₃₋₆ cycloaliphatic groups is optionally and independently substituted by up to six instances of halogen, oxo, C₁₋₄ alkyl, C₁₋₄ haloalkyl, —CN, C₁₋₄ alkoxy, —N(R¹⁰)₂ or C₁₋₄ haloalkoxy;
each occurrence of R′ is independently selected from hydrogen, C₁₋₄ alkyl, C₁₋₄ haloalkyl, C₃₋₆ cycloalkyl or —C(O)(C₁₋₄ alkyl);
each occurrence of R¹⁶ is independently selected from hydrogen, a C₁-C₄ aliphatic, a C₁-C₄ haloaliphatic, a C₃₋₇ cycloaliphatic or a 3-7-membered heterocyclyl; or two R¹⁶ groups attached to the same nitrogen atom, together with the nitrogen atom to which they are attached, form a 3-7-membered heterocycle; wherein each instance of R¹⁶ and each cycle formed by two R¹⁶ groups is optionally and independently substituted by up to 6 instances of halogen, —CN, oxo, C₁₋₄ alkyl, C₁₋₄ haloalkyl, —N(R¹⁰)₂, C₁₋₄ alkoxy or C₁₋₄ haloalkoxy;
each occurrence of R¹⁰ is independently selected from hydrogen or C₁₋₄ alkyl; or 2 instances of R¹⁰ attached to the same nitrogen atom, which, together with the nitrogen atom to which they are attached, form a 3-7-membered heterocyclic ring;
X² is selected from N or CR²;
R² is selected from hydrogen, halogen, —CN, —NO₂, —CO₂R¹⁷, —C(O)N(R¹⁷)₂, a C₁₋₆ alkyl, —O(C₁₋₄ alkyl) or a C₃₋₆ cycloaliphatic ring; wherein said alkyl, —O(alkyl) or cycloaliphatic ring is optionally and independently substituted with up to 6 instances of halogen, —CN, oxo, —O(C₁₋₂ alkyl) or —O(C₁₋₂ haloalkyl);
each R¹⁷ is independently selected from hydrogen, a C₁₋₄ alkyl, C₁₋₄ haloalkyl, C₃₋₆ cycloalkyl, phenyl or a 5-6-membered heteroaryl;
ring A is selected from a benzene ring or a 5-6-membered heteroaryl ring having 1-3 heteroatoms independently selected from N, O or S;
m is an integer selected from 0, 1, 2 or 3;
each occurrence of R^{A} is independently selected from halogen, —NO₂, —CN, oxo, —OR¹³, —S(O)₂R′³, —SO₂N(R¹³)₂, —N(R¹³)₂, —C(O)OR¹³, —C(O)R′³, —N(R¹³)C(O)R¹³, —N(R¹³)S(O)₂R¹³, —C(O)N(R¹³)₂, —OC(O)R¹³ or a aliphatic; wherein each of said aliphatic groups is optionally and independently substituted with up to four instances of R¹⁸;
each occurrence of R¹³ is independently selected from hydrogen, a C₁-C₄ aliphatic, a C₃₋₇ cycloaliphatic or a 3-7-membered heterocyclyl; wherein each of said aliphatic, cycloaliphatic and heterocyclyl groups is independently and optionally substituted with up to 6 instances of R¹⁸; or two instances of R¹³ attached to the same nitrogen atom, together with the nitrogen atom to which they are attached, form a 3-7-membered heterocycle; wherein each of said heterocycle rings is optionally and independently substituted with up to 6 instances of R¹⁸; provided that when R^{A} is a substituent on a nitrogen atom it can also be one of the R¹ groups defined above as possible substituents on the pyrrole ring nitrogen;
ring C is benzene or a 5-6-membered heteroaryl ring having 1 to 3 heteroatoms independently selected from N, O or S;
ring C is fused to ring A;
p is an integer selected from 0, 1, 2 or 3;
each occurrence of R^{C} is independently selected from halogen, —NO₂, —CN, oxo, —OR¹⁴, —SR¹⁴, —S(O)₂R¹⁴, —SO₂N(R¹⁴)₂, —N(R¹⁴)₂, —C(O)OR¹⁴, —C(O)R¹⁴, —N(R¹⁴)C(O)R¹⁴, —N(R¹⁴)S(O)₂R¹⁴, —C(O)N(R¹⁴)₂, —OC(O)R¹⁴ or a C₁₋₄ aliphatic; wherein each of said aliphatic groups is optionally and independently substituted with up to four instances of R¹⁸; or two instances of R^{C} attached to two vicinal ring C atom(s), together with the ring atom(s) to which they are attached, form a 3-7-membered heterocycle or C₃₋₇ cycloaliphatic; wherein said cycloaliphatic or heterocyclic rings are independently and optionally substituted with up to 6 instances of halogen, —CN, —OH, C₁₋₂ alkyl, C₁₋₂ haloalkyl, C₁₋₂ alkoxy or C₁₋₂ haloalkoxy;
provided that at least one of the bicyclic system formed by ring B and the fused pyrrole or pyrazole moiety, or the bicyclic system formed by rings A and C, consists of two fused 5-membered rings;
each R¹⁴ is independently selected from hydrogen, a C₁₋₄ aliphatic, a C₃₋₇ cycloaliphatic or a 3-7-membered heterocyclyl; wherein each of said aliphatic, cycloaliphatic and heterocyclyl groups is independently and optionally substituted with up to 6 instances of halogen; or two instances of R¹⁴ attached to the same or different atom(s), together with the atom(s) to which they are attached, form a 3-7-membered heterocycle; wherein said heterocycle is optionally and independently substituted with up to 6 instances of halogen, CN, OH, C₁₋₂ alkyl, C₁₋₂ haloalkyl, C₁₋₂ alkoxy or C₁₋₂ haloalkoxy;
each occurrence of R¹⁸ is independently selected from halogen, —OR¹⁹, —SR¹⁹, —CN, —OCOR¹⁹, —CO₂R¹⁹, —C(O)N(R¹⁹)₂, —N(R¹⁹)C(O)R¹⁹, —N(R¹⁹)₂, a C₁₋₄ aliphatic, a C₁₋₄ haloaliphatic, a C₃₋₆ cycloaliphatic or a 3-6-membered heterocyclyl; wherein each of said cycloaliphatic and heterocyclyl rings is optionally and independently substituted with up to 6 instances of halogen, —CN, —OH, C₁₋₂ alkyl, C₁₋₂ haloalkyl, C₁₋₂ alkoxy or C₁₋₂ haloalkoxy;
each occurrence of R¹⁹ is independently selected from hydrogen, a C₁₋₄ aliphatic or a C₁₋₄ haloaliphatic;
ring B is benzene, a 5-6-membered heteroaryl ring containing up to 3 heteroatoms selected from N, O and S, or a 5-8-membered carbocyclic or heterocyclic ring; wherein said benzene, heteroaryl, carbocyclic or heterocyclic ring is optionally and independently substituted with up to 6 instances of halogen, —CN, —OH, C₁₋₂ alkyl, C₁₋₂ haloalkyl, C₁₋₂ alkoxy or C₁₋₂ haloalkoxy;
n is an integer selected from 0, 1, 2 or 3;
each occurrence of R^{B} is independently selected from halogen, —CN, —NO₂, oxo, —C(O)NR^{X}, —C(O)OR^{X}, a C₁₋₄ aliphatic or a C₃₋₆ cycloaliphatic; wherein each of said aliphatic and cycloaliphatic groups is independently and optionally substituted with up to 6 instances of halogen, —CN, —OH, oxo, —O(C₁₋₂ alkyl), —O(C₁₋₂ haloalkyl), —C₁₋₂ alkyl or —C₁₋₂ haloalkyl; and
each R^{X} is independently selected from hydrogen, a C₁₋₄ alkyl, C₁₋₄ haloalkyl, C₃₋₆ cycloalkyl, phenyl or a 5-6-membered heteroaryl.

In another aspect, this disclosure provides pharmaceutical compositions comprising a compound of Formula I, or a pharmaceutically acceptable salt thereof, and a pharmaceutically acceptable carrier, vehicle or adjuvant.

In a third aspect, these compounds, and pharmaceutically acceptable compositions thereof, are useful, either alone or in combination therapy, for treating or lessening the severity of a variety of disorders in a patient. These disorders include but are not limited to pain, including acute, chronic, inflammatory, post-operative, cancer and neuropathic pain; immune disorders, including autoimmune disorders; inflammation; disorders that have an inflammatory component; emesis; and liver fibrosis.

### BRIEF DESCRIPTION OF THE FIGURES

FIG. 1 shows a nuclear magnetic resonance spectrum of compound I-76 using the parameters described therein.

FIG. 2 shows a nuclear magnetic resonance spectrum of compound I-87 using the parameters described therein.

FIG. 3 shows a nuclear magnetic resonance spectrum of compound I-35 using the parameters described therein.

FIG. 4 shows a nuclear magnetic resonance spectrum of compound I-65 using the parameters described therein.

The figures are provided by way of examples and are not intended to limit the scope of the present invention.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### Description of Exemplary Compounds

### Definitions and General Terminology

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in Organic Chemistry, Thomas Sorrell, University Science Books, Sausalito: 1999, and March's Advanced Organic Chemistry, 5^{th} Ed., Smith, M. B. and March, J., eds. John Wiley & Sons, New York: 2001, which are herein incorporated by reference in their entirety.

As described herein, compounds of the invention may optionally be substituted with one or more substituents, such as illustrated generally below, or as exemplified by particular classes, subclasses, and species of the invention. The phrase “optionally substituted” is used interchangeably with the phrase “substituted or unsubstituted.” In general, the term “substituted” refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. If a substituent radical or structure is not identified or defined as “optionally substituted”, the substituent radical or structure is not substituted. As it will be apparent to one of ordinary skill in the art, groups such as H, halogen, —NO₂, —CN, —OH, —NH₂ or —OCF₃ would not be substitutable groups.

The phrase “up to”, as used herein, refers to zero or any integer number that is equal or less than the number following the phrase. For example, “up to 3” means any one of 0, 1, 2, or 3. As described herein, a specified number range of atoms includes any integer therein. For example, a group having from 1-4 atoms could have 1, 2, 3 or 4 atoms. It will be understood by one of ordinary skill in the art that when a group is characterized as substituted (as opposed to optionally substituted) with, e.g., “up to 3” substituents, it can only be substituted with 1, 2 or 3 substituents.

When any variable occurs more than one time at any position, its definition on each occurrence is independent from every other occurrence.

Selection of substituents and combinations envisioned by this disclosure are only those that result in the formation of stable or chemically feasible compounds. Such choices and combinations will be apparent to those of ordinary sill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. In some embodiments, a stable compound or chemically feasible compound is one that is not substantially altered when kept at a temperature of 25° C. or less, in the absence of moisture or other chemically reactive conditions, for at least a week.

A compound, such as the compounds of the invention or other compounds herein disclosed, may be present in its free form (e.g., an amorphous form, a crystalline form or polymorphs). Under certain conditions, compounds may also form salts, and/or other multi-component crystalline forms (e.g., solvates, hydrates and co-crystals). As used herein, the term co-form is synonymous with the term multi-component crystalline form. When one of the components in the co-form has clearly transferred a proton to the other component, the resulting co-form is referred to as a “salt”. When both compounds in a multi-component crystalline form are independently solids at room temperature, the resulting co-form is referred to as a “co-crystal”. In co-crystals, no proton transfer takes place between the different components of the co-form. The formation of a salt or a co-crystal is determined by the size of the difference in the pKas between the partners that form the mixture. As used herein, a “solvate” refers to an association or complex of one or more solvent molecules and a compound disclosed herein (or its salts or co-crystals). A “hydrate” is a particular type of solvate in which the solvent is water. Examples of solvents that can form solvates include, but are not limited to: water, isopropanol, ethanol, methanol, (dimethyl sulfoxide) DMSO, ethyl acetate, acetic acid, ethanolamine, tetrahydrofuran (THF), dichloromethane (DCM), N,N-dimethylformamide (DMF).

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are within the scope of the disclosure.

The present disclosure also embraces isotopically labeled compounds that are identical to those recited herein, but for the fact that one or more atoms are replaced by an atom having an atomic mass or mass number different from the atomic mass or mass number usually found in nature. All isotopes of any particular atom or element as specified are contemplated as being within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron-emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The term “aliphatic” or “aliphatic group”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”. In some instances the term can be used in the phrase “aromatic carbocycle”, and in this case it refers to an “aryl group” as defined below.

The term “cycloaliphatic” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, Spiro or bridged. In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3-7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

“Heterocycle” (or “heterocyclyl” or “heterocyclic”), as used herein, refers to a ring system in which one or more ring members are an independently selected heteroatom, which is completely saturated or that contains one or more units of unsaturation but which is not aromatic, and which has a single point of attachment to the rest of the molecule. Unless otherwise specified, throughout this disclosure heterocycle is used as a synonym of “non-aromatic heterocycle”). In some instances the term can be used in the phrase “aromatic heterocycle”, and in this case it refers to a “heteroaryl group” as defined below. The term heterocycle also includes fused, spiro or bridged heterocyclic ring systems. Unless otherwise specified, a heterocycle may be monocyclic, bicyclic or tricyclic. In some embodiments, the heterocycle has 3-18 ring members in which one or more ring members is a heteroatom independently selected from oxygen, sulfur or nitrogen, and each ring in the system contains 3 to 7 ring members. In other embodiments, a heterocycle may be a monocycle having 3-7 ring members (2-6 carbon atoms and 1-4 heteroatoms) or a bicycle having 7-10 ring members (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is in the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl.

As defined herein, the term “benzene” ring, used alone or as part of a larger moiety, refers to a six-membered aromatic carbocyclic ring. A benzene ring can be either isolated or fused to another ring.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. A bicyclic 6,5 heteroaromatic system, as used herein, for example, is a six-membered heteroaromatic ring fused to a second five-membered ring wherein the radical or point of attachment is on the six-membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl; and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tri-cyclic ring systems including cycloaliphatic, heterocyclic, aryl or heteroaryl, each of which has been previously defined.

“Fused” bicyclic ring systems comprise two rings which share two adjoining ring atoms.

“Bridged” bicyclic ring systems comprise two rings which share three or four adjacent ring atoms. As used herein, the term “bridge” refers to a bond or an atom or a chain of atoms connecting two different parts of a molecule. The two atoms that are connected through the bridge (usually but not always, two tertiary carbon atoms) are referred to as “bridgeheads.” Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl.

“Spiro” bicyclic ring systems share only one ring atom (usually a quaternary carbon atom).

The term “ring atom” refers to an atom such as C, N, O or S that is part of the ring of an aromatic group, a cycloaliphatic group or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example, N (as in 3,4-dihydro-2H-pyrrolyl), NH (as in pyrrolidinyl) or NR⁺ (as in N-substituted pyrrolidinyl).

Non-aromatic nitrogen-containing heterocyclic rings that are substituted on a ring nitrogen and attached to the remainder of the molecule at a ring carbon atom are said to be N-substituted. For example, an N-alkyl piperidinyl group is attached to the remainder of the molecule at the two, three or four position of the piperidinyl ring and substituted at the ring nitrogen with an alkyl group. Non-aromatic nitrogen-containing heterocyclic rings such as pyrazinyl that are substituted on a ring nitrogen atom and attached to the remainder of the molecule at a second ring nitrogen atom are said to be N′-substituted N-heterocycles. For example, an N′-acyl N-pyrazinyl group is attached to the remainder of the molecule at one ring nitrogen atom and substituted at the second ring nitrogen atom with an acyl group.

In some embodiments, two independent occurrences of a variable may be taken together with the atom(s) to which each variable is bound to form a 5-8-membered, heterocyclyl, aryl, or heteroaryl ring or a 3-8-membered cycloalkyl ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of —ORₒ as in Formula D1:

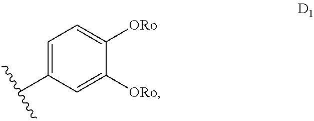

these two occurrences of —ORₒ are taken together with the carbon ring atoms to which they are bound to form a fused 6-membered oxygen containing ring as in Formula D2:

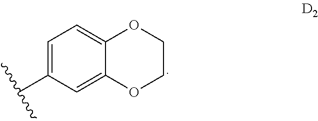 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

In some embodiments, an alkyl or aliphatic chain can be optionally interrupted with another atom or group. This means that a methylene unit of the alkyl or aliphatic chain can optionally be replaced with said other atom or group. Unless otherwise specified, the optional replacements form a chemically stable compound. Optional interruptions can occur both within the chain and/or at either end of the chain, i.e., both at the point of attachment(s) to the rest of the molecule and/or at the terminal end. Two optional replacements can also be adjacent to each other within a chain so long as it results in a chemically stable compound. Unless otherwise specified, if the replacement or interruption occurs at a terminal end of the chain, the replacement atom is bound to a H on the terminal end. For example, if —CH₂CH₂CH₃ were optionally interrupted with —O—, the resulting compound could be —OCH₂CH₃, —CH₂OCH₃, or —CH₂CH₂OH. In another example, if the divalent linker —CH₂CH₂CH₂— were optionally interrupted with —O—, the resulting compound could be —OCH₂CH₂—, —CH₂OCH₂—, or —CH₂CH₂O—. The optional replacements can also completely replace all of the carbon atoms in a chain. For example, a C₃ aliphatic can be optionally replaced by —N(R^{$})—, —C(O)—, and —N(R^{$})— to form —N(R^{$})C(O)N(R^{$})— (a urea).

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent at the end of the substituent bound to the rest of the chemical structure. Alkylcarboxy (e.g., alkyl-C(O)O— or alkyl-O(CO)—) and alkylcarboxyaryl (e.g., alkyl-C(O)O-aryl or alkyl-O(CO)-aryl-) are examples of carboxy groups used internally.

As described herein, a bond drawn from a substituent to the center of one ring within a multiple-ring system (as shown below), represents substitution of the substituent at any substitutable position in any of the rings within the multiple ring system. For example, formula D3 represents possible substitution in any of the positions shown in formula D4:

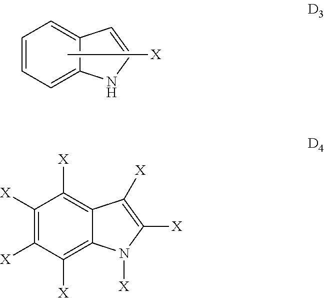

This also applies to multiple ring systems fused to optional ring systems (which would be represented by dotted lines). For example, in Formula D5, X is an optional substituent both for ring A and ring B.

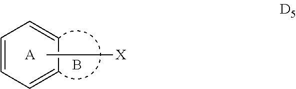

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

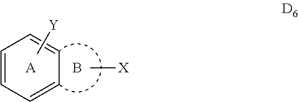

As used herein, the terms “alkoxy” or “alkylthio” refer to an alkyl group, as previously defined, attached to the molecule, or to another chain or ring, through an oxygen (“alkoxy,” i.e, —O-alkyl) or a sulfur (“alkylthio,” i.e., —S-alkyl) atom.

The terms Cₙ₋ₘ “alkoxyalkyl”, Cₙ₋ₘ “alkoxyalkenyl”, Cₙ₋ₘ“alkoxyaliphatic”, and Cₙ₋ₘ “alkoxyalkoxy” mean alkyl, alkenyl, aliphatic or alkoxy, as the case may be, substituted with one or more alkoxy groups, wherein the combined total number of carbons of the alkyl and alkoxy groups, alkenyl and alkoxy groups, aliphatic and alkoxy groups or alkoxy and alkoxy groups, combined, as the case may be, is between the values of n and m. For example, a C₄₋₆ alkoxyalkyl has a total of 4-6 carbons divided between the alkyl and alkoxy portion, e.g., —CH₂OCH₂CH₂CH₃, —CH₂CH₂OCH₂CH₃ or —CH₂CH₂CH₂OCH₃.

When the moieties described in the preceding paragraph are optionally substituted, they can be substituted in either or both of the portions on either side of the oxygen or sulfur. For example, an optionally substituted C₄ alkoxyalkyl could be, for instance, —CH₂CH₂OCH₂(Me)CH₃ or —CH₂(OH)OCH₂CH₂CH₃; a C₅ alkoxyalkenyl could be, for instance, —CH═CHOCH₂CH₂CH₃ or —CH═CHCH₂OCH₂CH₃.

The terms aryloxy, arylthio, benzyloxy or benzylthio, refer to an aryl or benzyl group attached to the molecule, or to another chain or ring, through an oxygen (“aryloxy”, “benzyloxy,” e.g., —O-Ph, —OCH₂Ph) or sulfur (“arylthio,” e.g., —S-Ph, —S—CH₂Ph) atom. Further, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or aliphatic will be indicated separately. Thus, a 5-6-membered aryloxy(C₁₋₄alkyl) is a 5-6-membered aryl ring, attached via an oxygen atom to a C₁₋₄ alkyl chain which, in turn, is attached to the rest of the molecule via the terminal carbon of the C₁₋₄ alkyl chain.

An “aralkyl” refers to an aryl ring attached to an alkyl chain, wherein the point of attachment is on the alkyl chain. Unless otherwise indicated, as used in this disclosure an optionally substituted aralkyl is optionally substituted only in the aryl portion. The same principle applies to, for example, an optionally substituted aralkoxy (i.e., an aryl ring attached to an alkoxy), which would be attached to the rest of the molecule through the oxygen of the alkoxy and substituted on the aryl portion. A substituted aryloxyalkyl would be attached to the rest of the molecule through the alkyl chain and substituted on the aryl ring, and the aryl and alky would, in turn, be attached to each other through an oxygen atom. For example, an optionally substituted 6-membered aryloxy(C₃alkyl) group could be, for instance, —(CH₃)CH₂-[p-(MeO)-Ph]; an optionally substituted 6-membered heteroaryloxy(C₄alkyl) could be, for instance, —CH₂CH₂CH₂CH₂—O-(3-F-2-pyridyl) or —CH(CH₃)—O—CH₂CH₂-(5,6-dimethyl-1,3-pyrimidine). An alkyl chain on the “aralkyl” group that is also optionally substituted will be specifically indicated. For instance, an optionally substituted 6-membered heteroaryloxy(C₄alkyl) that is also optionally substituted on the alkyl would be referred to as “an optionally substituted 6-membered heteroaryloxy(C₄alkyl), wherein said C₄ alkyl chain is optionally substituted.” An example of this latter group could be —CH(OH)—CF(CH₃)—CH₂—O-(5,6-dimethyl-1,3-pyrimidine), wherein the alkyl chain is substituted with F and with —OH.

As used herein, the terms “halogen” or “halo” mean F, Cl, Br, or I.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example, a C₁₋₃ haloalkyl could be —CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN or C≡N.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. For example a C₁₋₃ cyanoalkyl could be —C(CN)₂CH₂CH₃ and a C₁₋₂ cyanoalkenyl could be ═CHC(CN)H₂.

As used herein, an “amino” group refers to —NH₂.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups. For example a C₁₋₃ aminoalkyl could be —CH(NH₂)CH₂CH₂NH₂ and a C₁₋₂ aminoalkoxy could be —OCH₂CH₂NH₂.

The term “hydroxyl” or “hydroxy” refers to —OH.

The terms “hydroxyalkyl”, “hydroxyalkenyl”, “hydroxyaliphatic”, and “hydroxyalkoxy” mean alkyl, alkenyl, aliphatic or alkoxy, as the case may be, substituted with one or more —OH groups. For example a C₁₋₃ hydroxyalkyl could be —CH₂(CH₂OH)CH₃ and a C₄ hydroxyalkoxy could be —OCH₂C(CH₃)(OH)CH₃.

As used herein, an “aroyl” or “heteroaroyl” refers to an —C(O)-aryl or a —C(O)-heteroaryl. The aryl and heteroaryl portion of the aroyl or heteroaroyl is optionally substituted as previously defined.

As used herein, a “carbonyl”, used alone or in connection with another group refers to —C(O)— or —C(O)H. For example, as used herein, an “alkoxycarbonyl,” refers to a group such as —C(O)O(alkyl).

As used herein, an “oxo” refers to ═O, wherein oxo is usually, but not always, attached to a carbon atom. An aliphatic chain can be optionally interrupted by a carbonyl group or can optionally be substituted by an oxo group, and both expressions refer to the same, e.g., —CH₂—C(O)—CH₃.

As used herein, a “sulfonamide” group refers to the structure —S(O)₂—NR^{x}R^{y} or —NR^{x}—S(O)₂—R^{z} when used terminally; or —S(O)₂—NR^{x}— or —NR^{x}—S(O)₂— when used internally.

As used herein, a “sulfonyl” or “sulfone” group refers to —S(O)₂—R_{X} when used terminally and —S(O)₂— when used internally.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, in the context of resin chemistry (e.g., using solid resins or soluble resins or beads), the term “linker” refers to a bifunctional chemical moiety attaching a compound to a solid support or soluble support.

In all other situations, a “linker”, as used herein, refers to a divalent group in which the two free valences are on different atoms (e.g., carbon or heteroatom) or are on the same atom but can be substituted by two different substituents. For example, a methylene group can be C₁ alkyl linker (—CH₂—) which can be substituted by two different groups, one for each of the free valences (e.g., as in Ph-CH₂-Ph, wherein methylene acts as a linker between two phenyl rings). Ethylene can be C₂ alkyl linker (—CH₂CH₂—) wherein the two free valences are on different atoms. The amide group, for example, can act as a linker when placed in an internal position of a chain (e.g., —CONH—). A linker can be the result of interrupting an aliphatic chain by certain functional groups or of replacing methylene units on said chain by said functional groups. E.g., a linker can be a C₁₋₆ aliphatic chain in which up to two methylene units are substituted by —C(O)— or —NH— (as in —CH₂—NH—CH₂C(O)—CH₂— or —CH₂NH—C(O)—CH₂—). An alternative way to define the same —CH₂—NH—CH₂—C(O)CH₂— and —CH₂—NH—C(O)—CH₂— groups is as a C₃ alkyl chain optionally interrupted by up to two —C(O)— or —NH— moieties. Cyclic groups can also form linkers: e.g., a 1,6-cyclohexanediyl can be a linker between two R groups, as in

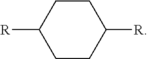

A linker can additionally be optionally substituted in any portion or position.

Divalent groups of the type R—CH═ or R₂C═, wherein both free valences are in the same atom and are attached the same substituent, are also possible. In this case, they will be referred to by their IUPAC accepted names. For instance an alkylidene (such as, for example, a methylidene (═CH₂) or a ethylidene (═CH—CH₃)) would not be encompassed by the definition of a linker in this disclosure.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W., Wuts, P. G in Protective Groups in Organic Synthesis, Third Edition, John Wiley & Sons, New York: 1999, the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified above, and certain exemplary nitrogen protecting groups are also detailed in Chapter 7 in Greene, T. W., Wuts, P. G in Protective Groups in Organic Synthesis, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

As used herein, “amide coupling agent” or “amide coupling reagent” means a compound that reacts with the hydroxyl moiety of a carboxy moiety thereby rendering it susceptible to nucleophilic attack. Exemplary amide coupling agents include DIC (diisopropylcarbodiimide), EDCI (1-Ethyl-3-(3-dimethylaminopropyl)carbodiimide), DCC (dicyclohexylcarbodiimide), BOP (Benzotriazol-1-yloxy-tris(dimethylamino)-phosphonium hexafluorophosphate), pyBOP ((Benzotriazol-1-yloxy)tripyrrolidinophosphonium Hexafluorophosphate), etc.

The compounds of the invention are defined herein by their chemical structures and/or chemical names. Where a compound is referred to by both a chemical structure and a chemical name, and the chemical structure and chemical name conflict, the chemical structure is determinative of the compound's identity.

In one aspect, the invention is a compound of Formula I, or a pharmaceutically acceptable salt thereof,

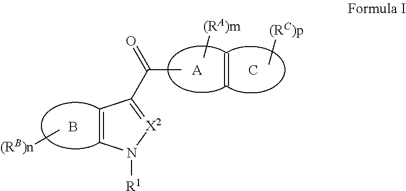

wherein
R¹ is -V-R⁸;
V is a single bond or a divalent linker between R⁸ and the nitrogen to which V is bonded, wherein said linker is a C₁-6 aliphatic; and wherein up to two saturated carbons of said C₁ aliphatic are optionally and independently replaced by —O—, —C(O)—, —C(S)—, —C(O)N(R)—, —N(R)C(O)—, —(R)NC(O)N(R)—, —C(O)O—, —OC(O)—, —N(R)—, —N(R)S(O)₂—, —S(O)₂N(R)—, N(R)S(O)₂N(R)— or —S(O)_{q}—;
each q is an integer independently selected from 0, 1 or 2;
each occurrence of R is independently selected from hydrogen, a C₁₋₄ alkyl, C₁ haloalkyl, C₃₋₆ cycloalkyl or —C(O)(C₁₋₄ alkyl);
R⁸ is selected from hydrogen, halogen, —CN, —NO₂, phenyl, a 5-6-membered heteroaryl ring, a C₃₋₁₀ cycloaliphatic ring or a 3-10-membered heterocyclyl ring; wherein each said phenyl, heteroaryl, cycloaliphatic or heterocyclyl ring is optionally and independently substituted with up to 6 instances of R¹⁵;
each occurrence of R¹⁵ is independently selected from halogen, oxo, —NO₂, —CN, —OR¹⁶, —N(R¹⁶)₂, —C(O)OR¹⁶, —C(O)R¹⁶, —N(R′)C(O)R¹⁶, —C(O)N(R¹⁶)₂, —OC(O)R¹⁶, —SR¹⁶, —S(O)₂R¹⁶, —SO₂N(R¹⁶)₂, —S(O)R¹⁶, a C₁₋₆ aliphatic or a C₃₋₆ cycloaliphatic; wherein each of said C₁₋₆ aliphatic and C cycloaliphatic groups is optionally and independently substituted by up to six instances of halogen, oxo, C₁₋₄ alkyl, C₁₋₄ haloalkyl, —CN, C₁₋₄ alkoxy, —N(R¹⁰)₂ or C₁₋₄ haloalkoxy;
each occurrence of R′ is independently selected from hydrogen, C₁₋₄ alkyl, C₁₋₄ haloalkyl, C₃₋₆ cycloalkyl or —C(O)(C₁₋₄ alkyl);
each occurrence of R¹⁶ is independently selected from hydrogen, a C₁-C₄ aliphatic, a C₁-C₄ haloaliphatic, a C₃₋₄ cycloaliphatic or a 3-7-membered heterocyclyl; or two R¹⁶ groups attached to the same nitrogen atom, together with the nitrogen atom to which they are attached, form a 3-7-membered heterocycle; wherein each instance of R¹⁶ and each cycle formed by two R¹⁶ groups is optionally and independently substituted by up to 6 instances of halogen, —CN, oxo, C₁₋₄ alkyl, C, haloalkyl, —N(R¹⁰)₂, C₁₋₄ alkoxy or C₁₋₄ haloalkoxy;
each occurrence of R¹⁰ is independently selected from hydrogen or C₁₋₄ alkyl; or 2 instances of R¹⁰ attached to the same nitrogen atom, which, together with the nitrogen atom to which they are attached, form a 3-7-membered heterocyclic ring;
X² is selected from N or CR²;
R² is selected from hydrogen, halogen, —CN, —NO₂, —CO₂R¹⁷, —C(O)N(R¹⁷)₂, a C₁₋₆ alkyl, —O(C₁₋₄ alkyl) or a C cycloaliphatic ring; wherein said alkyl, —O(alkyl) or cycloaliphatic ring is optionally and independently substituted with up to 6 instances of halogen, —CN, oxo, —O(C₁₋₂ alkyl) or —O(C₁₋₂ haloalkyl);
each R¹⁷ is independently selected from hydrogen, a C₁₋₄ alkyl, C₁₋₄ haloalkyl, C₃₋₆ cycloalkyl, phenyl or a 5-6-membered heteroaryl;
ring A is selected from a benzene ring or a 5-6-membered heteroaryl ring having 1-3 heteroatoms independently selected from N, O or S;
m is an integer selected from 0, 1, 2 or 3;
each occurrence of R^{A} is independently selected from halogen, —NO₂, —CN, oxo, —OR¹³, —SR¹³, —S(O)₂R¹³, —SO₂N(R¹³)₂, —N(R¹³)₂, —C(O)OR¹³, —C(O)R¹³, —N(R¹³)C(O)R¹³, —N(R¹³)S(O)₂R¹³, —C(O)N(R¹³)₂, —OC(O)R¹³ or a C, aliphatic; wherein each of said aliphatic groups is optionally and independently substituted with up to four instances of R¹⁸;
each occurrence of R¹³ is independently selected from hydrogen, a C₁-C₄ aliphatic, a C₃₋₇ cycloaliphatic or a 3-7-membered heterocyclyl; wherein each of said aliphatic, cycloaliphatic and heterocyclyl groups is independently and optionally substituted with up to 6 instances of R¹⁸; or two instances of R¹³ attached to the same nitrogen atom, together with the nitrogen atom to which they are attached, form a 3-7-membered heterocycle; wherein each of said heterocycle rings is optionally and independently substituted with up to 6 instances of R¹⁸; provided that when R^{A} is a substituent on a nitrogen atom it can also be one of the R¹ groups defined above as possible substituents on the pyrrole ring nitrogen;
ring C is benzene or a 5-6-membered heteroaryl ring having 1 to 3 heteroatoms independently selected from N, O or S;
ring C is fused to ring A;
p is an integer selected from 0, 1, 2 or 3;
each occurrence of R^{C} is independently selected from halogen, —NO₂, —CN, oxo, —OR¹⁴, —SR¹⁴, —S(O)₂R¹⁴, —SO₂N(R¹⁴)₂, —N(R¹⁴)₂, —C(O)OR¹⁴, —C(O)R¹⁴, —N(R¹⁴)C(O)R¹⁴, —N(R¹⁴)S(O)₂R¹⁴, —C(O)N(R¹⁴)₂, —OC(O)R¹⁴ or a C₁₋₄ aliphatic; wherein each of said aliphatic groups is optionally and independently substituted with up to four instances of R¹⁸; or two instances of R^{C} attached to two vicinal ring C atom(s), together with the ring atom(s) to which they are attached, form a 3-7-membered heterocycle or C₃₋₇ cycloaliphatic; wherein said cycloaliphatic or heterocyclic rings are independently and optionally substituted with up to 6 instances of halogen, —CN, —OH, C₁₋₂ alkyl, C₁₋₂ haloalkyl, C₁₋₂ alkoxy or C₁₋₂ haloalkoxy;
provided that at least one of the bicyclic system formed by ring B and the fused pyrrole or pyrazole moiety, or the bicyclic system formed by rings A and C, consists of two fused 5-membered rings;
each R¹⁴ is independently selected from hydrogen, a C₁₋₄ aliphatic, a C₃₋₇ cycloaliphatic or a 3-7-membered heterocyclyl; wherein each of said aliphatic, cycloaliphatic and heterocyclyl groups is independently and optionally substituted with up to 6 instances of halogen; or two instances of R¹⁴ attached to the same or different atom(s), together with the atom(s) to which they are attached, form a 3-7-membered heterocycle; wherein said heterocycle is optionally and independently substituted with up to 6 instances of halogen, —CN, —OH, C₁₋₂ alkyl, C₁₋₂ haloalkyl, C₁₋₂ alkoxy or C₁₋₂ haloalkoxy;
each occurrence of R¹⁸ is independently selected from halogen, —OR¹⁹, —SR¹⁹, —CN, —OCOR¹⁹, —CO₂R¹⁹, —C(O)N(R¹⁹)₂, —N(R¹⁹)C(O)R¹⁹, —N(R¹⁹)₂, a C₁₋₄ aliphatic, a C_{IA} haloaliphatic, a C₃₋₆ cycloaliphatic or a 3-6-membered heterocyclyl; wherein each of said cycloaliphatic and heterocyclyl rings is optionally and independently substituted with up to 6 instances of halogen, —CN, —OH, C₁₋₂ alkyl, C₁₋₂ haloalkyl, C₁₋₂ alkoxy or C₁₋₂ haloalkoxy;
each occurrence of R¹⁹ is independently selected from hydrogen, a C₁₋₄ aliphatic or a C₁₋₄ haloaliphatic;
ring B is benzene, a 5-6-membered heteroaryl ring containing up to 3 heteroatoms selected from N, O and S, or a 5-8-membered carbocyclic or heterocyclic ring; wherein said benzene, heteroaryl, carbocyclic or heterocyclic ring is optionally and independently substituted with up to 6 instances of halogen, —CN, —OH, C₁₋₂ alkyl, C₁₋₂ haloalkyl, C₁₋₂ alkoxy or C₁₋₂ haloalkoxy;
n is an integer selected from 0, 1, 2 or 3;
each occurrence of R¹³ is independently selected from halogen, —CN, —NO₂, oxo, —C(O)NR^{X}, —C(O)OR^{X}, a C₁₋₄ aliphatic or a C₃₋₆ cycloaliphatic; wherein each of said aliphatic and cycloaliphatic groups is independently and optionally substituted with up to 6 instances of halogen, —CN, —OH, oxo, —O(C₁₋₂alkyl), —O(C₁₋₂ haloalkyl), —C₁₋₂ alkyl or —C₁₋₂ haloalkyl; and
each Rx is independently selected from hydrogen, a C₁₋₄ alkyl, C₁₋₄ haloalkyl, C₃₋₆ cycloalkyl, phenyl or a 5-6-membered heteroaryl.

In one embodiment, V is a bond or a C₁₋₆ alkylene linker, wherein up to 2 methylene units of said alkylene linker are optionally and independently replaced by —O—, —C(O)—, —C(O)N(R)—, —N(R)C(O)—, —C(O)O—, —N(R)—, —N(R)S(O)₂—, —S(O)₂N(R)— or —S(O)₂—.

In another embodiment, R⁸ is selected from hydrogen, halogen, a C₃₋₁₀ cycloaliphatic group or a 3-10-membered heterocyclyl ring; wherein said cycloaliphatic or heterocyclyl ring is optionally and independently substituted with up to 6 instances of R¹⁵.

In another embodiment, R¹ is hydrogen.

In still another embodiment, R¹ is selected from the group consisting of:

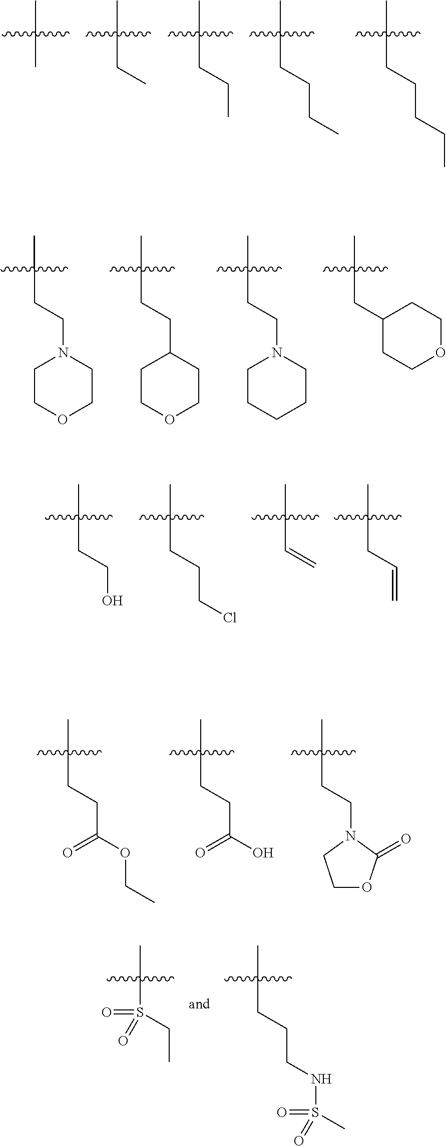

In one embodiment, Ring B is benzene, or a 5-6-membered heteroaryl ring containing up to 3 heteroatoms selected from N, O and S, or a 5-8-membered carbocyclic or heterocyclic ring; wherein said benzene, heteroaryl, carbocyclic or heterocyclic ring is optionally and independently substituted with up to 6 instances of halogen, —OH, C₁₋₂ alkyl or C₁₋₂ alkoxy.

In a further embodiment, Ring B is benzene and is optionally and independently substituted with up to 3 instances of halogen, —OH, C₁₋₂ alkyl or C₁₋₂ alkoxy.

In another embodiment, (Ring B)-(R^{B})n is

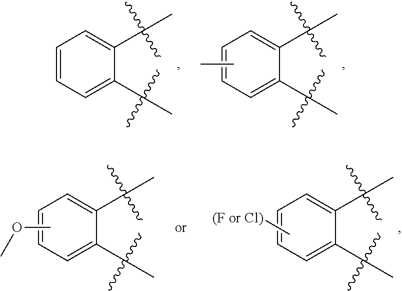

wherein the two broken lines represent the two points of attachment to the pyrrole or pyrazole ring.

In another embodiment, Ring B is a 5-6-membered heteroaryl ring containing up to 3 heteroatoms selected from N, O and S, and is optionally and independently substituted with up to 3 instances of halogen, —OH, C₁₋₂ alkyl or C₁₋₂ alkoxy.

In a further embodiment, Ring B is pyridine, optionally and independently substituted with up to 3 instances of halogen, —OH, C₁₋₂ alkyl or C₁₋₂ alkoxy.

In a further embodiment, Ring B is

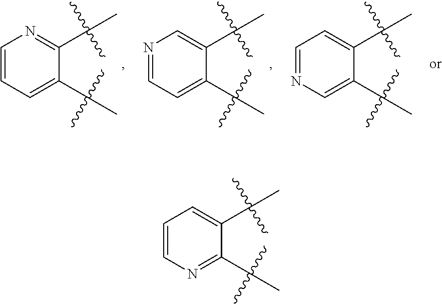 [SMILES: *c1cccnc1* ; *c1ccncc1* ; *c1ccncc1* ; *c1cccnc1*]

and is unsubstituted.

In one embodiment, Ring B is a thiophene, optionally and independently substituted with up to 3 instances of halogen, —OH, C₁₋₂ alkyl or C₁₋₂ alkoxy.

In a further embodiment, Ring B is

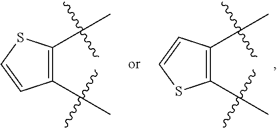 [SMILES: *c1ccsc1* ; *c1ccsc1*]

and is unsubstituted.

In another embodiment, Ring B is a 5-8-membered carbocyclic or heterocyclic ring, wherein said carbocyclic or heterocyclic ring is optionally and independently substituted with up to 3 instances of halogen, —OH, C₁₋₂ alkyl or C₁₋₂ alkoxy.

In another embodiment, (Ring B)-(R^{B})n is

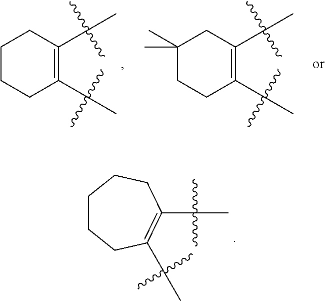

In one embodiment, X² is CR².

In a further embodiment, R² is hydrogen or C₁₋₆ alkyl.

In another embodiment, R² is hydrogen or methyl.

In one embodiment, Ring A is benzene, pyridine, thiophene or pyrrole and Ring A is fused to Ring C.

In another embodiment, each instance of R^{A} is independently selected from halogen or a C₁₋₄ aliphatic; wherein each of said aliphatic groups is optionally and independently substituted with up to two instances of a C₃₋₆ cycloaliphatic or a 3-6-membered heterocyclyl.

In one embodiment, Ring C is benzene, pyridine, furan, thiophene or pyrrole, and Ring C is fused to Ring A.

In another embodiment, each instance of R^{C} is independently selected from halogen, or a C₁₋₄ aliphatic; wherein each of said aliphatic groups is optionally and independently substituted with up to two instances of a C₃₋₆ cycloaliphatic or a 3-6-membered heterocyclyl.

In a further embodiment, the compound is selected from those depicted in the table below:

| | |
|---|---|
| 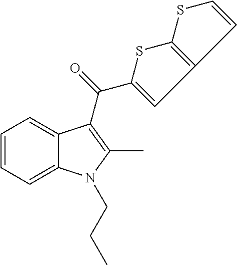 [SMILES: CCCn1c(C)c(C(=O)c2cc3ccsc3s2)c2ccccc21] | I-46 |
| 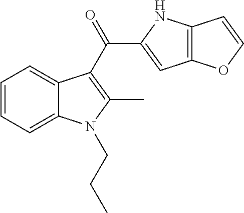 [SMILES: CCCn1c(C)c(C(=O)c2cc3occc3[nH]2)c2ccccc21] | I-71 |
| 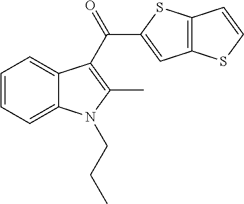 [SMILES: CCCn1c(C)c(C(=O)c2cc3sccc3s2)c2ccccc21] | I-20 |
| 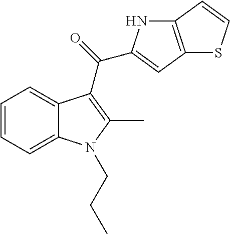 [SMILES: CCCn1c(C)c(C(=O)c2cc3sccc3[nH]2)c2ccccc21] | I-81 |
| 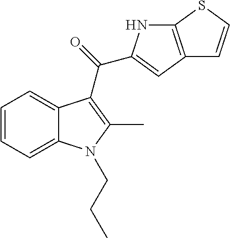 [SMILES: CCCn1c(C)c(C(=O)c2cc3ccsc3[nH]2)c2ccccc21] | I-74 |
| 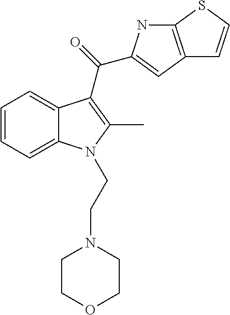 [SMILES: Cc1c(C(=O)c2cc3ccsc3[nH]2)c2ccccc2n1CCN1CCOCC1] | I-82 |
| 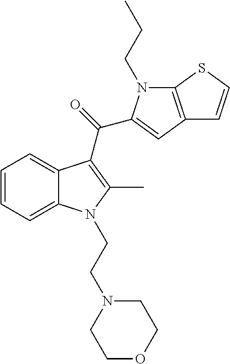 [SMILES: CCCn1c(C(=O)c2c(C)n(CCN3CCOCC3)c3ccccc23)cc2ccsc21] | I-73 |
| 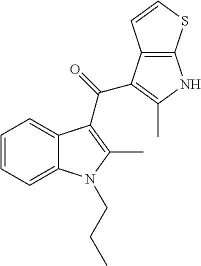 [SMILES: CCCn1c(C)c(C(=O)c2c(C)[nH]c3sccc23)c2ccccc21] | I-6 |
| 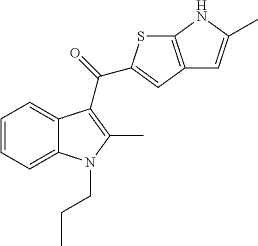 [SMILES: CCCn1c(C)c(C(=O)c2cc3cc(C)[nH]c3s2)c2ccccc21] | I-75 |
| 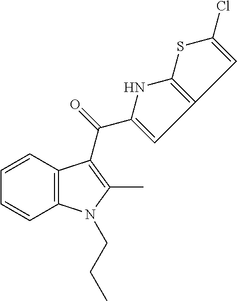 [SMILES: CCCn1c(C)c(C(=O)c2cc3cc(Cl)sc3[nH]2)c2ccccc21] | I-48 |
| 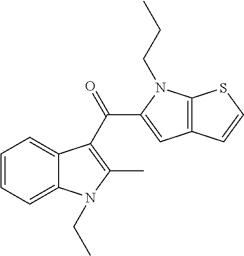 [SMILES: CCCn1c(C(=O)c2c(C)n(CC)c3ccccc23)cc2ccsc21] | I-50 |
| 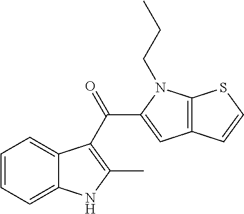 [SMILES: CCCn1c(C(=O)c2c(C)[nH]c3ccccc23)cc2ccsc21] | I-51 |
| 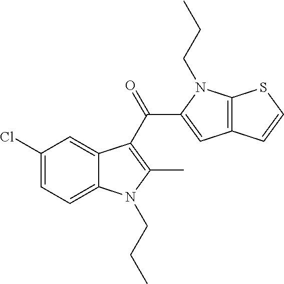 [SMILES: CCCn1c(C)c(C(=O)c2cc3ccsc3n2CCC)c2cc(Cl)ccc21] | I-52 |
| 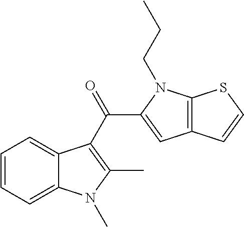 [SMILES: CCCn1c(C(=O)c2c(C)n(C)c3ccccc23)cc2ccsc21] | I-53 |
| 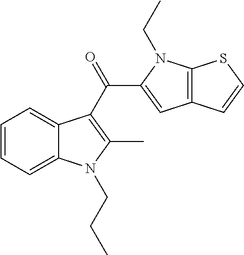 [SMILES: CCCn1c(C)c(C(=O)c2cc3ccsc3n2CC)c2ccccc21] | I-54 |
| 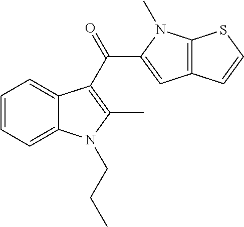 [SMILES: CCCn1c(C)c(C(=O)c2cc3ccsc3n2C)c2ccccc21] | I-55 |
| 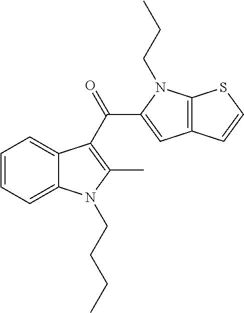 [SMILES: CCCCn1c(C)c(C(=O)c2cc3ccsc3n2CCC)c2ccccc21] | I-58 |
| 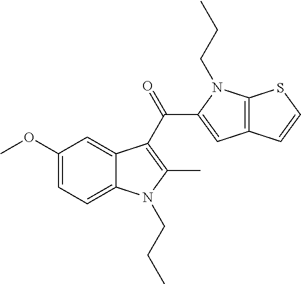 [SMILES: CCCn1c(C)c(C(=O)c2cc3ccsc3n2CCC)c2cc(OC)ccc21] | I-61 |
| 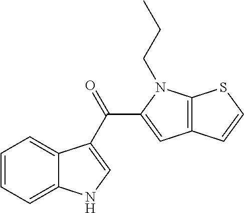 [SMILES: CCCn1c(C(=O)c2c[nH]c3ccccc23)cc2ccsc21] | I-64 |
| 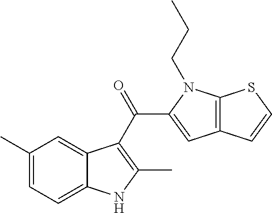 [SMILES: CCCn1c(C(=O)c2c(C)[nH]c3ccc(C)cc23)cc2ccsc21] | I-68 |
| 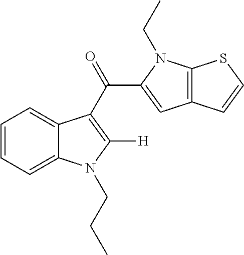 [SMILES: CCCn1cc(C(=O)c2cc3ccsc3n2CC)c2ccccc21] | I-70 |
| 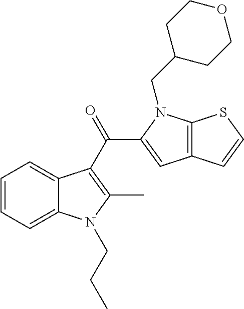 [SMILES: CCCn1c(C)c(C(=O)c2cc3ccsc3n2CC2CCOCC2)c2ccccc21] | I-72 |
| 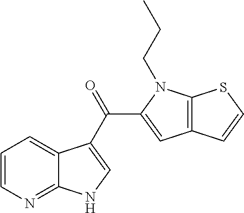 [SMILES: CCCn1c(C(=O)c2c[nH]c3ncccc23)cc2ccsc21] | I-43 |
| 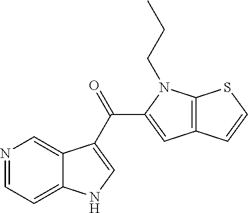 [SMILES: CCCn1c(C(=O)c2c[nH]c3ccncc23)cc2ccsc21] | I-44 |
| 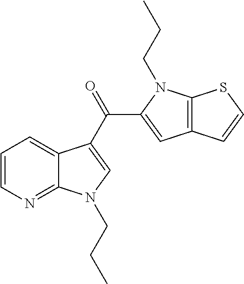 [SMILES: CCCn1cc(C(=O)c2cc3ccsc3n2CCC)c2cccnc21] | I-45 |
| 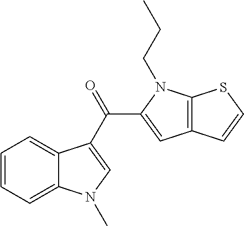 [SMILES: CCCn1c(C(=O)c2cn(C)c3ccccc23)cc2ccsc21] | I-47 |
| 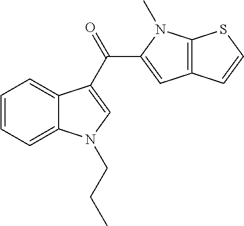 [SMILES: CCCn1cc(C(=O)c2cc3ccsc3n2C)c2ccccc21] | I-32 |
| 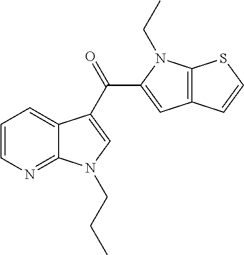 [SMILES: CCCn1cc(C(=O)c2cc3ccsc3n2CC)c2cccnc21] | I-34 |
| 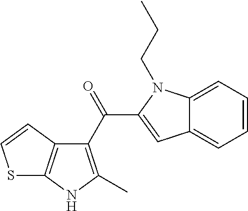 [SMILES: CCCn1c(C(=O)c2c(C)[nH]c3sccc23)cc2ccccc21] | I-13 |
| 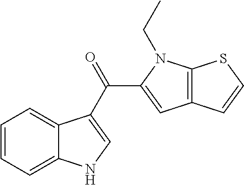 [SMILES: CCn1c(C(=O)c2c[nH]c3ccccc23)cc2ccsc21] | I-37 |
| 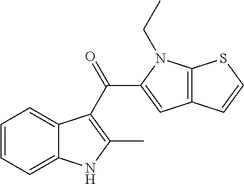 [SMILES: CCn1c(C(=O)c2c(C)[nH]c3ccccc23)cc2ccsc21] | I-38 |
| 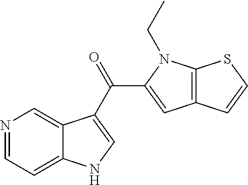 [SMILES: CCn1c(C(=O)c2c[nH]c3ccncc23)cc2ccsc21] | I-39 |
| 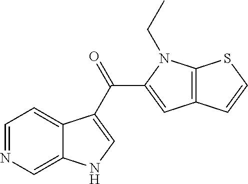 [SMILES: CCn1c(C(=O)c2c[nH]c3cnccc23)cc2ccsc21] | I-40 |
| 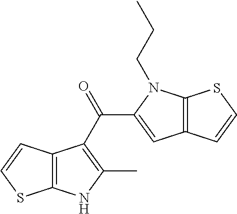 [SMILES: CCCn1c(C(=O)c2c(C)[nH]c3sccc23)cc2ccsc21] | I-84 |
| 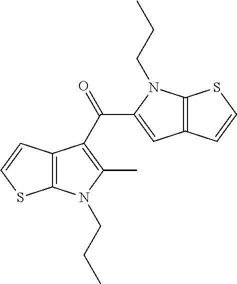 [SMILES: CCCn1c(C(=O)c2c(C)n(CCC)c3sccc23)cc2ccsc21] | I-83 |
| 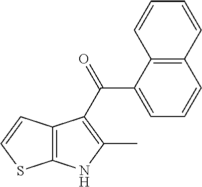 [SMILES: Cc1[nH]c2sccc2c1C(=O)c1cccc2ccccc12] | I-3 |
| 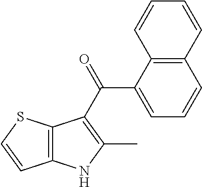 [SMILES: Cc1[nH]c2ccsc2c1C(=O)c1cccc2ccccc12] | I-4 |
| 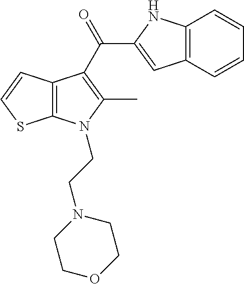 [SMILES: Cc1c(C(=O)c2cc3ccccc3[nH]2)c2ccsc2n1CCN1CCOCC1] | I-10 |
| 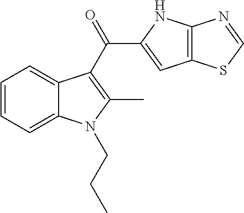 [SMILES: CCCn1c(C)c(C(=O)c2cc3scnc3[nH]2)c2ccccc21] | I-30 |
| 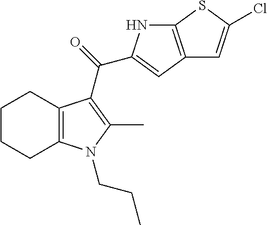 [SMILES: CCCn1c(C)c(C(=O)c2cc3cc(Cl)sc3[nH]2)c2c1CCCC2] | I-76 |
| 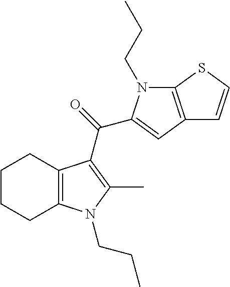 [SMILES: CCCn1c(C)c(C(=O)c2cc3ccsc3n2CCC)c2c1CCCC2] | I-77 |
| 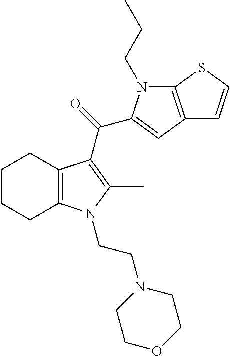 [SMILES: CCCn1c(C(=O)c2c3c(n(CCN4CCOCC4)c2C)CCCC3)cc2ccsc21] | I-79 |
| 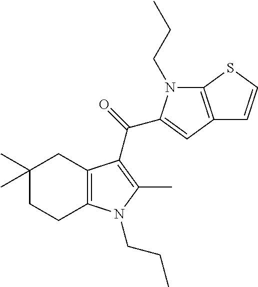 [SMILES: CCCn1c(C)c(C(=O)c2cc3ccsc3n2CCC)c2c1CCC(C)(C)C2] | I-35 |
| 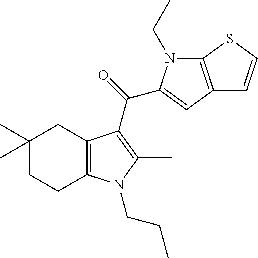 [SMILES: CCCn1c(C)c(C(=O)c2cc3ccsc3n2CC)c2c1CCC(C)(C)C2] | I-65 |
| 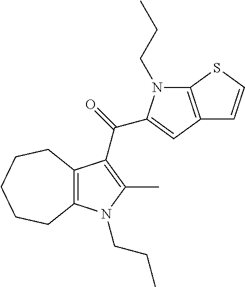 [SMILES: CCCn1c(C)c(C(=O)c2cc3ccsc3n2CCC)c2c1CCCCC2] | I-80 |
| 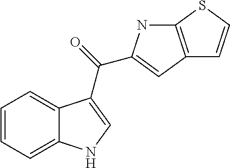 [SMILES: O=C(c1cc2ccsc2[nH]1)c1c[nH]c2ccccc12] | I-29 |
| 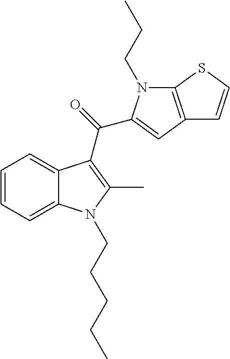 [SMILES: CCCCCn1c(C)c(C(=O)c2cc3ccsc3n2CCC)c2ccccc21] | I-60 |
| 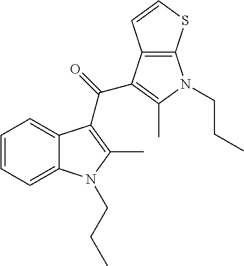 [SMILES: CCCn1c(C)c(C(=O)c2c(C)n(CCC)c3sccc23)c2ccccc21] | I-7 |
| 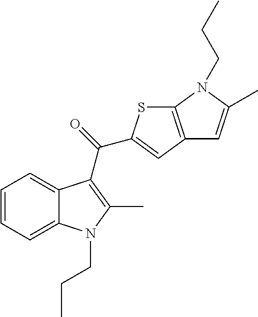 [SMILES: CCCn1c(C)c(C(=O)c2cc3cc(C)n(CCC)c3s2)c2ccccc21] | I-63 |
| 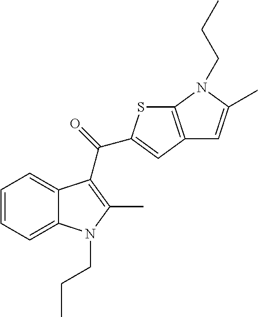 [SMILES: CCCn1c(C)c(C(=O)c2cc3cc(C)n(CCC)c3s2)c2ccccc21] | I-56 |
| 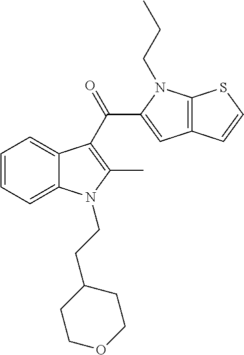 [SMILES: CCCn1c(C(=O)c2c(C)n(CCC3CCOCC3)c3ccccc23)cc2ccsc21] | I-57 |
| 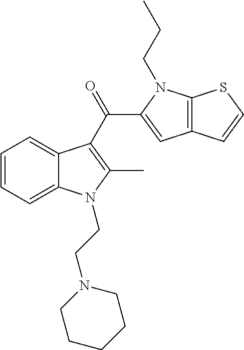 [SMILES: CCCn1c(C(=O)c2c(C)n(CCN3CCCCC3)c3ccccc23)cc2ccsc21] | I-59 |
| 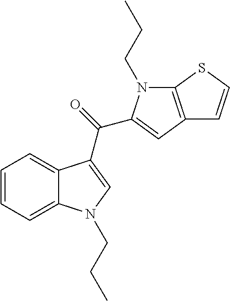 [SMILES: CCCn1cc(C(=O)c2cc3ccsc3n2CCC)c2ccccc21] | I-62 |
| 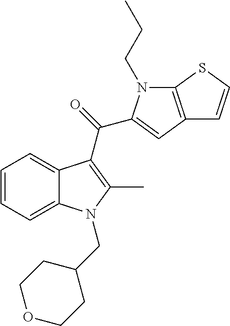 [SMILES: CCCn1c(C(=O)c2c(C)n(CC3CCOCC3)c3ccccc23)cc2ccsc21] | I-66 |
| 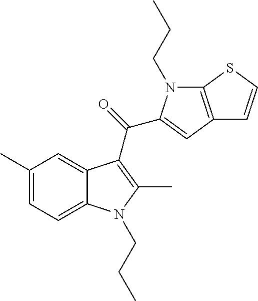 [SMILES: CCCn1c(C)c(C(=O)c2cc3ccsc3n2CCC)c2cc(C)ccc21] | I-69 |
| 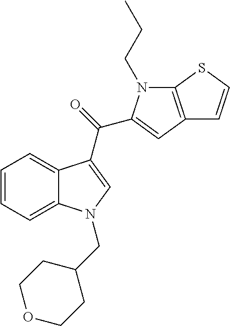 [SMILES: CCCn1c(C(=O)c2cn(CC3CCOCC3)c3ccccc23)cc2ccsc21] | I-41 |
| 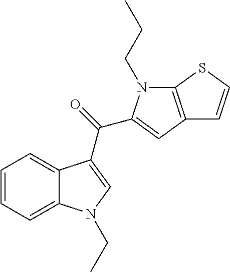 [SMILES: CCCn1c(C(=O)c2cn(CC)c3ccccc23)cc2ccsc21] | I-42 |
| 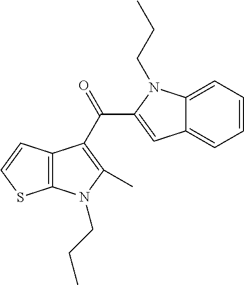 [SMILES: CCCn1c(C(=O)c2c(C)n(CCC)c3sccc23)cc2ccccc21] | I-14 |
| 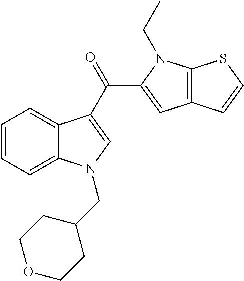 [SMILES: CCn1c(C(=O)c2cn(CC3CCOCC3)c3ccccc23)cc2ccsc21] | I-16 |
| 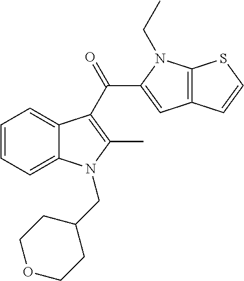 [SMILES: CCn1c(C(=O)c2c(C)n(CC3CCOCC3)c3ccccc23)cc2ccsc21] | I-17 |
| 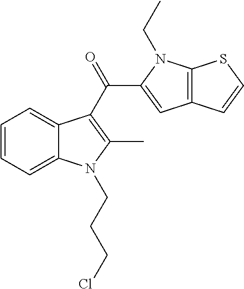 [SMILES: CCn1c(C(=O)c2c(C)n(CCCCl)c3ccccc23)cc2ccsc21] | I-18 |
| 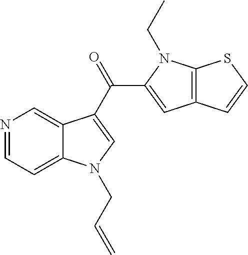 [SMILES: C=CCn1cc(C(=O)c2cc3ccsc3n2CC)c2cnccc21] | I-21 |
| 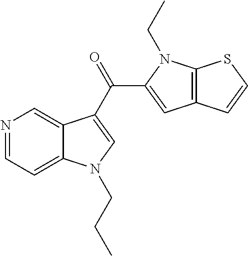 [SMILES: CCCn1cc(C(=O)c2cc3ccsc3n2CC)c2cnccc21] | I-22 |
| 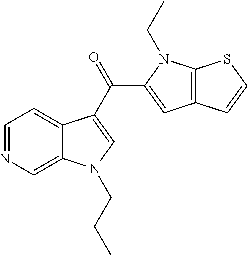 [SMILES: CCCn1cc(C(=O)c2cc3ccsc3n2CC)c2ccncc21] | I-23 |
| 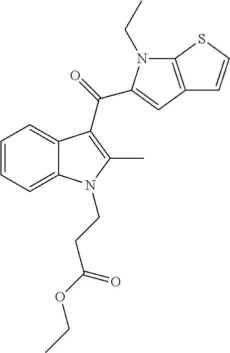 [SMILES: CCOC(=O)CCn1c(C)c(C(=O)c2cc3ccsc3n2CC)c2ccccc21] | I-26 |
| 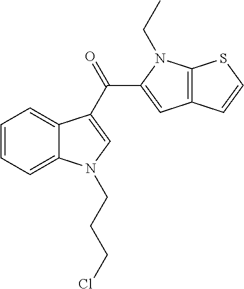 [SMILES: CCn1c(C(=O)c2cn(CCCCl)c3ccccc23)cc2ccsc21] | I-29 |
| 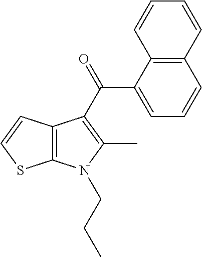 [SMILES: CCCn1c(C)c(C(=O)c2cccc3ccccc23)c2ccsc21] | I-2 |
| 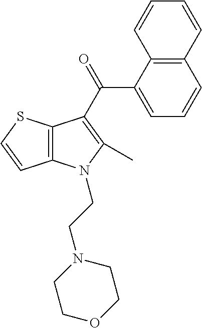 [SMILES: Cc1c(C(=O)c2cccc3ccccc23)c2sccc2n1CCN1CCOCC1] | I-1 |
| 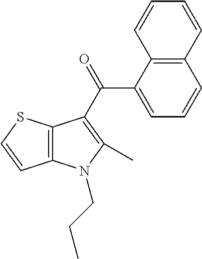 [SMILES: CCCn1c(C)c(C(=O)c2cccc3ccccc23)c2sccc21] | I-12 |
| 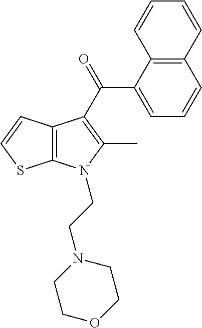 [SMILES: Cc1c(C(=O)c2cccc3ccccc23)c2ccsc2n1CCN1CCOCC1] | I-5 |
| 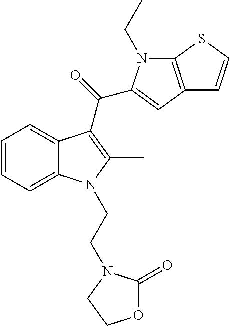 [SMILES: CCn1c(C(=O)c2c(C)n(CCN3CCOC3=O)c3ccccc23)cc2ccsc21] | I-25 |
| 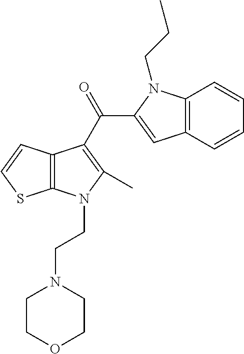 [SMILES: CCCn1c(C(=O)c2c(C)n(CCN3CCOCC3)c3sccc23)cc2ccccc21] | I-11 |
| 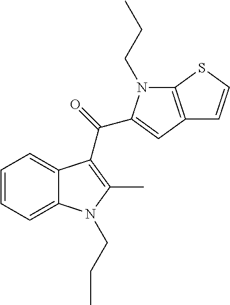 [SMILES: CCCn1c(C)c(C(=O)c2cc3ccsc3n2CCC)c2ccccc21] | I-78 |
| 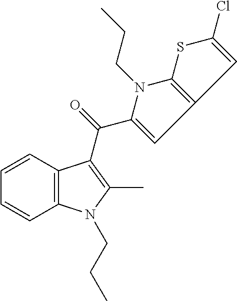 [SMILES: CCCn1c(C)c(C(=O)c2cc3cc(Cl)sc3n2CCC)c2ccccc21] | I-49 |
| 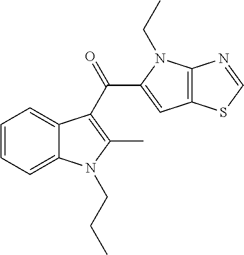 [SMILES: CCCn1c(C)c(C(=O)c2cc3scnc3n2CC)c2ccccc21] | I-31 |
| 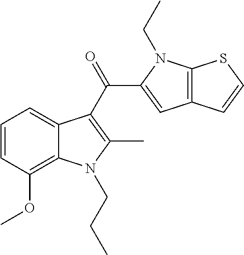 [SMILES: CCCn1c(C)c(C(=O)c2cc3ccsc3n2CC)c2cccc(OC)c21] | I-36 |
| 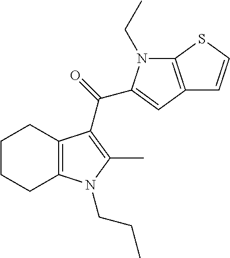 [SMILES: CCCn1c(C)c(C(=O)c2cc3ccsc3n2CC)c2c1CCCC2] | I-85 |
| 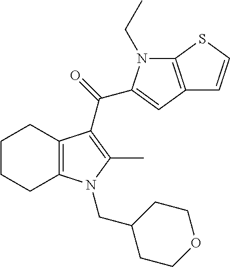 [SMILES: CCn1c(C(=O)c2c3c(n(CC4CCOCC4)c2C)CCCC3)cc2ccsc21] | I-86 |
| 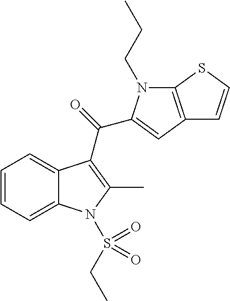 [SMILES: CCCn1c(C(=O)c2c(C)n(S(=O)(=O)CC)c3ccccc23)cc2ccsc21] | I-67 |
| 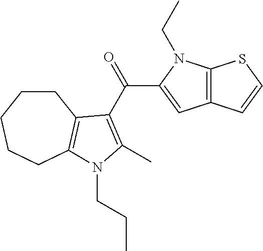 [SMILES: CCCn1c(C)c(C(=O)c2cc3ccsc3n2CC)c2c1CCCCC2] | I-87 |
| 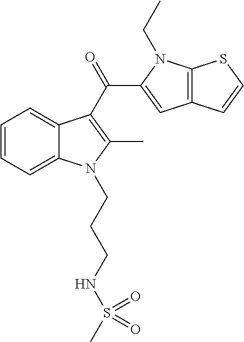 [SMILES: CCn1c(C(=O)c2c(C)n(CCCNS(C)(=O)=O)c3ccccc23)cc2ccsc21] | I-19 |
| 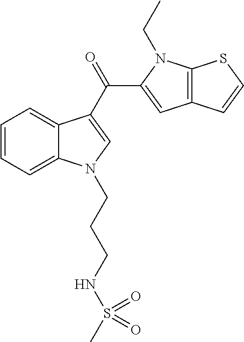 [SMILES: CCn1c(C(=O)c2cn(CCCNS(C)(=O)=O)c3ccccc23)cc2ccsc21] | I-28 |
| 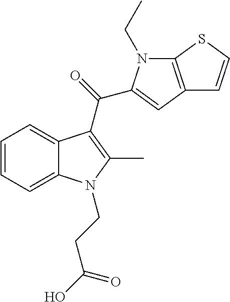 [SMILES: CCn1c(C(=O)c2c(C)n(CCC(=O)O)c3ccccc23)cc2ccsc21] | I-27 |
| 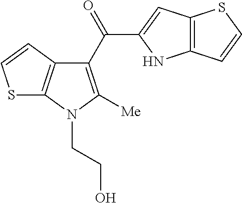 [SMILES: Cc1c(C(=O)c2cc3sccc3[nH]2)c2ccsc2n1CCO] | I-8 |
| 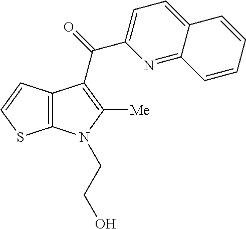 [SMILES: Cc1c(C(=O)c2ccc3ccccc3n2)c2ccsc2n1CCO] | I-9 |
| 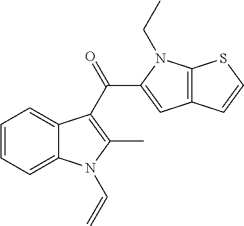 [SMILES: C=Cn1c(C)c(C(=O)c2cc3ccsc3n2CC)c2ccccc21] | I-24 |

### Pharmaceutically Acceptable Salts, Co-Forms and Pro-Drugs of the Invention.

The phrase “pharmaceutically acceptable salt,” as used herein, refers to pharmaceutically acceptable organic or inorganic salts of a compound of Formula I. For use in medicine, the salts of the compounds of Formula I will be pharmaceutically acceptable salts. Other salts may, however, be useful in the preparation of the compounds of Formula I or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound of Formula I is acidic, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of Formula I is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated herein by reference in its entirety.

In addition to the compounds described herein and their pharmaceutically acceptable salts, pharmaceutically acceptable solvates (e.g., hydrates) and co-crystals of these compounds and salts may also be employed in compositions to treat or prevent the herein identified disorders.

As used herein, the term “pharmaceutically acceptable solvate,” is a solvate formed from the association of one or more pharmaceutically acceptable solvent molecules to one of the compounds described herein. As used herein, the term “hydrate” means a compound described herein or a salt thereof that further includes a stoichiometric or non-stoichiometric amount of water bound by non-covalent intermolecular forces. The term solvate includes hydrates (e.g., hemihydrate, monohydrate, dihydrate, trihydrate, tetrahydrate, and the like).

“Pharmaceutically acceptable co-crystals” result when a pharmaceutically active compound crystallizes with another material (e.g., a carboxylic acid, a 4,4′-bipyridine or an excipient) that is also a solid at room temperature. Some pharmaceutically acceptable excipients are described in the next section. Other pharmaceutically acceptable substances that can be used to form co-crystals are exemplified by the GRAS (Generally regarded as safe) list of the US FDA.

In addition to the compounds described herein, pharmaceutically acceptable pro-drugs of these compounds may also be employed in compositions to treat or prevent the herein-identified disorders.

A “pharmaceutically acceptable pro-drug” includes any pharmaceutically acceptable ester, salt of an ester or other derivative or salt thereof of a compound described herein that, upon administration to a recipient, is capable of providing, either directly or indirectly, a compound described herein. Particularly favoured pro-drugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species. The term “pro-drug” encompasses a derivative of a compound that can hydrolyze, oxidize, or otherwise react under biological conditions (in vitro or in vivo) to provide a compound described herein. Examples of pro-drugs include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of pro-drugs include derivatives of compounds that comprise —NO, —NO₂, —ONO, or —ONO₂ moieties. Pro-drugs can typically be prepared using well-known methods, such as those described by Burger's Medicinal Chemistry and Drug Discovery (1995) 172-178, 949-982 (Manfred E. Wolff ed., 5th ed).

### Pharmaceutical Compositions and Methods of Administration.

The compounds herein disclosed, and their pharmaceutically acceptable salts, solvates, co-crystals and pro-drugs thereof may be formulated as pharmaceutical compositions or “formulations”.

A typical formulation is prepared by mixing a compound of Formula I, or a pharmaceutically acceptable salt, solvate, co-crystal or pro-drug thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of Formula I is being formulated. Solvents are generally selected based on solvents recognized by persons skilled in the art as safe (e.g., on the GRAS—Generally Regarded as Safe list) to be administered to a human or other mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g., enteric or slow release) preservatives, antioxidants, opaquing agents, glidants, processing aids, colorants, sweeteners, perfuming agents, flavoring agents and other known additives to provide an elegant presentation of the drug (e.g., a compound of Formula I or pharmaceutical composition thereof) or aid in the manufacturing of the pharmaceutical product (e.g., a medicament).

The formulations may be prepared using conventional dissolution and mixing procedures. For example, the bulk drug substance (e.g., compound of Formula I, a pharmaceutically acceptable salt, solvate, co-crystal or pro-drug thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8. When the agent described herein is a solid amorphous dispersion formed by a solvent process, additives may be added directly to the spray-drying solution when forming the mixture, for instance, the additive is dissolved or suspended in the solution as a slurry which can then be spray dried. Alternatively, the additives may be added following the spray-drying process to aid in the forming of the final formulated product.

The compound of Formula I or a pharmaceutically acceptable salt, solvate, co-crystal or pro-drug thereof is typically formulated into pharmaceutical dosage forms to provide an easily controllable dosage of the drug and to enable patient compliance with the prescribed regimen. Pharmaceutical formulations of compounds of Formula I, or a pharmaceutically acceptable salt, solvate, co-crystal or pro-drug thereof, may be prepared for various routes and types of administration. Various dosage forms may exist for the same compound, since different medical conditions may warrant different routes of administration.

The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. As a general proposition, the initial pharmaceutically effective amount of the inhibitor administered will be in the range of about 0.01-100 mg/kg per dose, namely about 0.1 to 20 mg/kg of patient body weight per day, with the typical initial range of compound used being 0.3 to 15 mg/kg/day.

The term “therapeutically effective amount” as used herein means an amount of active compound or pharmaceutical agent that elicits the biological or medicinal response in a tissue, system, animal or human that is being sought by a researcher, veterinarian, medical doctor or other clinician. The therapeutically or pharmaceutically effective amount of the compound to be administered will be governed by such considerations, and is the minimum amount necessary to ameliorate, cure or treat the disease or disorder or one or more of its symptoms.

The pharmaceutical compositions of Formula I will be formulated, dosed, and administered in a fashion, i.e., an amount, concentration, schedule, course, vehicle, and route of administration consistent with good medical practice. Factors for consideration in this context include the particular disorder being treated, the particular human or other mammal being treated, the clinical condition of the individual patient, the cause of the disorder, the site of delivery of the agent, the method of administration, the scheduling of administration, and other factors known to medical practitioners, such as the age, weight, and response of the individual patient.

The term “prophylactically effective amount” refers to an amount effective in preventing or substantially lessening the chances of acquiring a disease or disorder or in reducing the severity of the disease or disorder or one or more of its symptoms before it is acquired or before the symptoms develop. Roughly, prophylactic measures are divided between primary prophylaxis (to prevent the development of a disease) and secondary prophylaxis (whereby the disease has already developed and the patient is protected against worsening of this process).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively; in colloidal drug-delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”. In general, one can provide for controlled release of the agents described herein through the use of a wide variety of polymeric carriers and controlled release systems including erodible and non-erodible matrices, osmotic control devices, various reservoir devices, enteric coatings and multiparticulate control devices.

“Sustained-release preparations” are the most common applications of controlled release. Suitable examples of sustained-release preparations include semipermeable matrices of solid hydrophobic polymers containing the compound, in which the matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent deaggregation to fine particules. This provides a larger surface area exposed to the dissolution medium, resulting in a faster dissolution rate.

Agents described herein can be incorporated into an erodible or non-erodible polymeric matrix controlled-release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use. One ingredient of this water-swollen matrix is the water-swellable, erodible, or soluble polymer, which may generally be described as an osmopolymer, hydrogel or water-swellable polymer. Such polymers may be linear, branched, or crosslinked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g., chitin, chitosan, dextran and pullulan; gum agar, gum arabic, gum karaya, locust bean gum, gum tragacanth, carrageenans, gum ghatti, guar gum, xanthan gum and scleroglucan), starches (e.g., dextrin and maltodextrin), hydrophilic colloids (e.g., pectin), phosphatides (e.g., lecithin), alginates (e.g., ammonium alginate, sodium, potassium or calcium alginate, propylene glycol alginate), gelatin, collagen, and cellulosics. Cellulosics are cellulose polymers that have been modified by reaction of at least a portion of the hydroxyl groups on the saccharide repeat units with a compound to form an ester-linked or an ether-linked substituent. For example, the cellulosic ethyl cellulose has an ether-linked ethyl substituent attached to the saccharide repeat unit, while the cellulosic cellulose acetate has an ester-linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), carboxymethyl ethyl cellulose (CMEC), hydroxyethyl cellulose (HEC), hydroxypropyl cellulose (HPC), cellulose acetate (CA), cellulose propionate (CP), cellulose butyrate (CB), cellulose acetate butyrate (CAB), cellulose acetate propionate (CAP), cellulose acetate trimelliate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC). In certain embodiments, the cellulosics comprise various grades of low viscosity (MW less than or equal to 50,000 daltons, for example, the Dow Methocel™ series E5, E15LV, E50LV and K100LY) and high viscosity (MW greater than 50,000 daltons, for example, E4MCR, EIOMCR, K4M, K15M and K100M and the Methocel™ K series) HPMC. Other commercially available types of HPMC include the Shin Etsu Metolose 90SH series.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGITO, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

Alternatively, the agents of the present invention may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g methyl acrylate-methyl methacrylate copolymers, polyvinyl chloride, polyethylene), hydrophilic polymers (e.g., ethyl cellulose, cellulose acetate, crosslinked polyvinylpyrrolidone (also known as crospovidone)), and fatty compounds (e.g., carnauba wax, microcrystalline wax, and triglycerides). Such devices are described further in Remington: The Science and Practice of Pharmacy, 20th edition (2000).

As noted above, the agents described herein may also be incorporated into an osmotic control device. Such devices generally include a core containing one or more agents as described herein and a water-permeable, non-dissolving and non-eroding coating surrounding the core which controls the influx of water into the core from an aqueous environment of use so as to cause drug release by extrusion of some or all of the core to the environment of use. In certain embodiments, the coating is polymeric, aqueous-permeable, and has at least one delivery port. The core of the osmotic device optionally includes an osmotic agent that acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head). Non-limiting examples of osmotic control devices are disclosed in U.S. patent application Ser. No. 09/495,061.

The amount of water-swellable hydrophilic polymers present in the core may range from about 5 to about 80 wt % (including for example, 10 to 50 wt %). Non-limiting examples of core materials include hydrophilic vinyl and acrylic polymers, polysaccharides such as calcium alginate, polyethylene oxide (PEO), polyethylene glycol (PEG), polypropylene glycol (PPG), poly (2-hydroxyethyl methacrylate), poly (acrylic) acid, poly (methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolat. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to thereby effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Nonlimiting examples of standard disintegrants include materials such as sodium starch glycolate (e.g., Explotab™ CLV), microcrystalline cellulose (e.g., Avicel™), microcrystalline silicified cellulose (e.g., ProSoIv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 1-25% of the core agent.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP 378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers that are water permeable or impermeable, providing that the membrane deposited on the tablet core is porous or contains water-soluble porosigens or possesses a macroscopic hole for water ingress and drug release. Embodiments of this class of sustained-release devices may also be multilayered, as described, for example, in EP 378404.

When an agent described herein is a liquid or oil, such as a lipid vehicle formulation, for example as described in WO 05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

As further noted above, the agents described herein may be provided in the form of microparticulates, generally ranging in size from about 10 μm to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art. Such multiparticulates may be made by any known process, such as wet- and dry-granulation processes, extrusion/spheronization, roller-compaction, melt-congealing, or by spray-coating seed cores. For example, in wet- and dry-granulation processes, the agent described herein and optional excipients may be granulated to form multiparticulates of the desired size.

The agents can be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (co-emulsifer) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The compounds described herein can be incorporated into pharmaceutically acceptable nanoparticle, nanosphere, and nanocapsule formulations (Delie and Blanco-Prieto, 2005, Molecule 10:65-80). Nanocapsules can generally entrap compounds in a stable and reproducible way (Henry-Michelland et al., 1987, Int. J. Pharm. 35:121; Quintanar-Guerrero et al., 1998, Pharm. Res. 15:1056; Douglas et al., 1987, Crit. Rev. Ther. Drug Carrier Syst., 3:233-261). To avoid side effects due to intracellular polymeric overloading, ultrafine particles (sized around 0.1 μm) can be designed using polymers able to be degraded in vivo (e.g., biodegradable polyalkyl-cyanoacrylate nanoparticles). Such particles are described in the prior art (Couvreur et al., 1980, J. Pharm. Res., 69(2):199-202; Couvreur et al 1988, Crit. Rev. Ther. Drug Carrier Syst., 5:1-20; zur Muhlen et al., 1998, Eur. J. Pharm. Biopharm. 45:149-155; Zambaux et al. 1998, J. Control. Rel. 50:31-40; Pinto-Alphandry et al., 1995, Int. J. Antimicrob. Agents, 13:155-168 and U.S. Pat. No. 5,145,684).

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g., using capsules, powders, solutions, suspensions, tablets, sublingual tablets and the like), by inhalation (e.g., with an aerosol, gas, inhaler, nebulizer or the like), to the ear (e.g., using ear drops), topically (e.g., using creams, gels, liniments, lotions, ointments, pastes, transdermal patches, etc), ophthalmically (e.g., with eye drops, ophthalmic gels, ophthalmic ointments), rectally (e.g., using enemas or suppositories), nasally, buccally, vaginally (e.g., using douches, intrauterine devices, vaginal suppositories, vaginal rings or tablets, etc), via an implanted reservoir or the like, or parenterally depending on the severity and type of the disease being treated. The term “parenteral” as used herein includes, but is not limited to, subcutaneous, intravenous, intramuscular, intra-articular, intra-synovial, infrasternal, intrathecal, intrahepatic, intralesional and intracranial injection or infusion techniques. Preferably, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginici acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time-delay material such as glyceryl monostearate or glyceryl distearate alone or with a wax may be employed. A water-soluble taste-masking material such as hydroxypropyl-methylcellulose or hydroxypropyl-cellulose may be employed.

Formulations of a compound of Formula I that are suitable for oral administration may be prepared as discrete units such as tablets, pills, troches, lozenges, aqueous or oil suspensions, dispersible powders or granules, emulsions, hard or soft capsules, e.g., gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water-soluble carrier such as polyethyleneglycol or an oil medium, for example peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g., for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a longchain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of injectable formulations.

Oily suspensions may be formulated by suspending the compound of Formula I in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds of Formula I contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long-chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacteria-retaining filter, or by incorporating sterilizing agents in the form of sterile solid compositions that can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot-injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers, such as cocoa butter, beeswax, polyethylene glycol or a suppository wax that are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate-controlling membrane or by dispersing the compound in a polymer matrix or gel. Topical application for the lower intestinal tract can be effected in a rectal suppository formulation (see above) or in a suitable enema formulation. Topically transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds of Formula I may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds of Formula I include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a mean particle size for example in the range of 0.1 to 500 microns (including particles with a mean particle size in a range between 0.1 and 500 microns in increments of 0.5, 1, 30, 35 microns, etc.), and may be administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

In another aspect, a compound of Formula I or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Therapeutic Methods

The terms, “disease”, “disorder”, and “condition” may be used interchangeably here to refer to a CB receptor-mediated medical or pathological condition.

As used herein, the terms “subject” and “patient” are used interchangeably. The terms “subject” and “patient” refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

The term “biological sample”, as used herein, refers to an in vitro or ex vivo sample, and includes, without limitation, cell cultures or extracts thereof; biopsied material obtained from a mammal or extracts thereof; blood, saliva, urine, faeces, semen, tears, lymphatic fluid, ocular fluid, vitreous humour, or other body fluids or extracts thereof.

“Treat”, “treating” or “treatment” with regard to a disorder or disease refers to alleviating or abrogating the cause and/or the effects of the disorder or disease. As used herein, the terms “treat”, “treatment” and “treating” refer to the reduction or amelioration of the progression, severity and/or duration of a CB receptor-mediated condition, or the amelioration of one or more symptoms (preferably, one or more discernible symptoms) of said condition, resulting from the administration of one or more therapies (e.g., one or more therapeutic agents such as a compound or composition of the invention). In specific embodiments, the terms “treat”, “treatment” and “treating” refer to the amelioration of at least one measurable physical parameter of a CB2 receptor-mediated condition. In other embodiments the terms “treat”, “treatment” and “treating” refer to the inhibition of the progression of a CB receptor-mediated condition, either physically by, e.g., stabilization of a discernible symptom, physiologically by, e.g., stabilization of a physical parameter, or both.

As used herein, the terms “prevent”, “preventing” and “prevention” with regard to a disorder or disease refer to averting the cause and/or effects of a disease or disorder prior to the disease or disorder manifesting itself. The terms “prophylaxis” or “prophylactic use”, as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease.

The term “chemotherapy” refers to the use of medications, e.g., small molecule drugs (rather than “vaccines”) for treating a disorder or disease.

The term “chemoprophylaxis” refers to the use of medications, e.g., small molecule drugs (rather than “vaccines”) for the prevention of a disorder or disease.

In one embodiment, the methods of the invention are a preventative or “pre-emptive” measure to a patient, preferably a human, having a predisposition to developing a CB receptor-related disease or symptom.

The compounds and pharmaceutical compositions described herein can be used alone or in combination therapy for the treatment or prevention of pain. The pain can be chronic pain, acute pain, perioperative pain (e.g., associated with surgery), postoperative pain, visceral pain, inflammatory pain, cancer pain, headache pain, neuropathic pain, dental pain (such as odontalgia), bone pain, joint pain (e.g., osteoarthritis or rheumatoid arthritis), myofascial pain (e.g., muscular injury, fibromyalgia), labor pain, pain associated with injuries, trauma, allergies, dermatitis, immunodeficiency, Hodgkin's disease, Myasthenia gravis, nephrotic syndrome, scleroderma, or thyroiditis, central and peripheral pathway mediated pain, or pain associated with or the result of injury or age.

Neuropathic pain can be associated with neuronal lesions such as those induced by diabetes, HIV, herpes infection, or stroke. Chronic pain can result from injury and/or inflammation and includes chronic lower back pain, as well as pain from osteoarthritis or rheumatoid arthritis. Acute pain includes, for example, traumatic pain (e.g., bony fracture pain, sprains, strains and soft tissue damage), muscle pain, burn pain, and sunburn pain. Neuropathic pain can be associated with, for example, nerve injury, head trauma, hyperalgesia, allodynia, sciatica, amputation, trigeminal neuralgia, chemotherapeutic neuropathy, AIDS-related neuropathy, diabetic neuropathy, painful traumatic mononeuropathy, painful polyneuropathy, multiple sclerosis, root avulsions, postthoracotomy syndrome, central nervous system injury, non-herpetic neuralgia and post herpetic neuralgia. Neuropathic pain also includes lower back pain, toxin induced pain, chemotherapy induced pain, phantom limb pain, thalamic pain syndrome, post-stroke pain, stump pain, repetitive motion pain, pain induced by post-mastectomy syndrome.

Visceral pain includes, for example, pain associated with pancreatitis, peptic ulcer, interstitial cystitis, renal colic, angina, dysmenorrhoea, menstruation, irritable bowel syndrome (IBS), myocardial ischemia, and non-ulcer dyspepsia. Visceral pain also includes gynecological pain, non-cardiac chest pain, and chronic pelvic pain.

Inflammatory pain includes, for example, pain induced by or associated with disorders such as osteoarthritis, rheumatic fever, rheumatoid arthritis, rheumatic disease, tendonitis, juvenile arthritis, spondylitis, gouty arthritis, psoriatic arthritis, interstitial cystitis, peripheral neuritis, mucositis, fibromyalgia, pancreatitis, enteritis, cellulites, bony fractures, post-operative ileus, irritable bowel syndrome, Crohn's Disease, ulcerative colitis, cholecystitis, teno-synovitis, gout, vulvodynia, fibromyalgia, sprains and strains, systemic lupus erythematosus, myositis, and influenza and other viral infections such as the common cold. Inflammatory pain also includes sympathetically maintained pain, pain due to venomous and non-venomous snake bite, spider bite or insect sting, sports injury pain, myofascial pain (muscular injury, fibromyalgia), muscoskeletal pain, and pain due to inflammatory bowel diseases.

Cancer pain can be induced by or associated with tumors such as lymphatic leukemia, Hodgkin's disease, malignant lymphoma, lymphogranulomatoses, lymphosarcoma, solid malignant tumors, and extensive metastases.

Headache pain includes cluster headache, migraine with and without aura, tension type headache, headaches caused by injury or infection, hangovers, and headaches with unknown origins.

The compounds and pharmaceutical compositions described herein can be used alone or in combination therapy for the treatment or prevention of autoimmune disorders including, for example, alopecia greata (also known as systemic sclerosis (SS)), amyloses, amyotrophic lateral sclerosis, ankylosing spondylarthritis, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CMS), chronic inflammatory demyelinating polyneuropathy (CIPD), cicatricial pemphigold, cold agglutinin disease, connective tissue diseases, crest syndrome, Crohn's disease, Degos' disease, dermatomyositis-juvenile, discoid lupus, essential mixed cryoglobulinemia, fibromyalgia-fibromyositis, graft vs. host disease, transplantation rejection, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, lupus erythematosus, Meniere's disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, rectional arthritis, Reiter's syndrome, rheumatic fever, rheumatoid arthritis, sarcoidosis, scleroderma (progressive systemic sclerosis (PSS)), Sjogren's syndrome, stiff-man syndrome, systemic lupus erythematosus, Takayasu arteritis, temporal arteritis/giant cell arteritis, ulcerative colitis, undifferentiated spondylarthritis, uveitis, vitiligo, and Wegener's granulomatosis.

The compounds and pharmaceutical compositions described herein can be used alone or in combination therapy for the treatment or prevention of inflammatory disorders, including, for example, chronic and acute inflammatory disorders. Examples of disorders with inflammatory components include asthma, atopic allergy, allergy, atherosclerosis, bronchial asthma, eczema, glomerulonephritis, graft vs. host disease, hemolytic anemia, osteoarthritis, sepsis, septic shock (e.g., as antihypovolemic and/or antihypotensive agents), stroke, transplantation of tissue and organs, vasculitis, diabetic retinopathy and ventilator induced lung injury. The compounds and pharmaceutical compositions described herein can also be used alone or in combination therapy for the treatment or prevention of disease-states or indications that are accompanied by inflammatory processes such as:

(1) Lung diseases: e.g., asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral- or bacterial exacerbation of asthma, other non-allergic asthmas and “wheezy-infant syndrome”, pneumoconiosis, including aluminosis, anthracosis, asbestosis, chalicosis, ptilosis, siderosis, silicosis, tabacosis and byssinosis;

(2) Rheumatic diseases or autoimmune diseases or musculoskeletal diseases: e.g., all forms of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis, gout (metabolic arthritis); collagenoses of any genesis, e.g., systemic lupus erythematosus, scleroderma, polymyositis, dermatomyositis, Sjogren syndrome, Still disease, Felty syndrome; and osteoporosis and other bone resorption diseases;

(3) Allergic diseases including all forms of allergic reactions, e.g., allergic rhinitis, allergic conjunctivitis infectious parasitic, angioneurotic edema, hay fever, insect bites, allergic reactions to drugs, blood derivatives, contrast agents, etc., anaphylactic shock (anaphylaxis), urticaria, angioneurotic edema, delayed or immediate hypersensitivity, and contact dermatitis;

(4) Vascular diseases: e.g., panarteritis nodosa, polyarteritis nodosa, periarteritis nodosa, arteritis temporalis, Wegner granulomatosis, giant cell arthritis, atherosclerosis, reperfusion injury and erythema nodosum;

(5) Dermatological diseases: e.g., dermatitis, psoriasis, sunburn, burns, and eczema;

(6) Renal, urinary and pancreatic diseases: e.g., nephrotic syndrome and all types of nephritis (such as glomerulonephritis); pancreatitis; bladder hyperrelexia following bladder inflammation;

(7) Hepatic diseases: e.g., acute liver cell disintegration; acute hepatitis of various genesis (such as viral, toxic, drug-induced) and chronically aggressive and/or chronically intermittent hepatitis, liver fibrosis associated with liver injury or disease, including fibrosis caused or exacerbated by alcoholic liver cirrhosis, chronic viral hepatitis, non-alcoholic steatohepatitis and primary liver cancer;

(8) Gastrointestinal diseases: e.g., inflammatory bowel diseases, irritable bowel syndrome, regional enteritis (Crohn's disease), colitis ulcerosa, gastritis, aphthous ulcer, celiac disease, regional ileitis, and gastroesophageal reflux disease;

(9) Neurodegenerative diseases: e.g., in the treatment of neurodegeneration following stroke, cardiac arrest, pulmonary bypass, traumatic brain injury, spinal cord injury or the like;

(10) Eye diseases: e.g., allergic keratitis, uveitis, or iritis, conjunctivitis, blepharitis, neuritis nervi optici, choroiditis, glaucoma and sympathetic ophthalmia;

(11) Diseases of the ear, nose, and throat (ENT) area: e.g., tinnitus, allergic rhinitis or hay fever, otitis externa, caused by contact eczema, infection, etc., and otitis media;

(12) Neurological diseases: e.g., brain edema, particularly tumor-related brain edema, multiple sclerosis, acute encephalomyelitis, meningitis, acute spinal cord injury, trauma, dementia, particularly degenerative dementia (including senile dementia, Alzheimer's disease, Parkinson's disease and Creutzfeldt-Jacob disease, Huntington's chorea, Pick's disease, motor neuron disease), vascular dementia (including multi-infarct dementia and dementia associated with intracranial space occupying lesions, infections and related conditions such as HIV infection), Guillain-Barre syndrome, myasthenia gravis, stroke, and various forms of seizures (such as nodding spasms);

(13) Blood diseases: e.g., acquired hemolytic anemia, aplastic anemia, and idiopathic thrombocytopenia;

(14) Tumor diseases: e.g., acute lymphatic leukemia, Hodgkin's disease, malignant lymphoma, lymphogranulomatoses, lymphosarcoma, solid malignant tumors, and extensive metastases;

(15) Endocrine diseases: e.g., endocrine opthalmopathy, endocrine orbitopathia, thyrotoxic crisis, Thyroiditis de Quervain, Hashimoto thyroiditis, Morbus Basedow, granulomatous thyroiditis, struma lymphomatosa, Graves disease, type I diabetes (such as insulin-dependent diabetes); Organ and tissue transplantations and graft-versus-host diseases;

(16) Severe states of shock: e.g., septic shock, anaphylactic shock, and systemic inflammatory response syndrome (SIRS); and

(17) Various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, granulocyte transfusion, sarcoidosis, gingivitis, pyrexia, edema resulting from trauma associated with burns, sprains or fracture, cerebral edema and angioedema, and diabetes (such as diabetic vasculopathy, diabetic neuropathy, diabetic retinopathy, post capillary resistance and diabetic symptoms associated with insulitis (e.g., hyperglycemia, diuresis, proteinuria and increased nitrite and kallikrein urinary excretion)).

The compounds and pharmaceutical compositions described herein can be used alone or in combination therapy for the treatment or prevention of substance abuse related syndromes, disorders or diseases including, for example, drug abuse and drug withdrawal. Abused substances can include alcohol, amphetamines, amphetamine-like substances, caffeine, cannabis, cocaine, hallucinogens, inhalants, opioids, nicotine (and/or tobacco products), heroin abuse, barbiturates, phencyclidine (or phencyclidine-like compounds), sedative-hypnotics, benzodiazepines, or combinations of any of the foregoing. The compounds and pharmaceutical compositions can also be used to treat withdrawal symptoms and substance-induced anxiety or mood disorder. In addition, they can be used to reduce tobacco craving; treat nicotine dependency, addiction, or withdrawal; or aid in the cessation or lessening of tobacco in a subject in need thereof.

The compounds and pharmaceutical compositions described herein can be used alone or in combination therapy for the treatment or prevention of psychiatric disorders, such as depression (including, but not limited to, major depressive disorder, bipolar depression, unipolar depression, single or recurrent major depressive episodes (e.g., with or without psychotic features, catatonic features, and/or melancholic features), postpartum onset, seasonal affective disorder, dysthymic disorders (e.g., with early or late onset and with or without atypical features), neurotic depression and social phobia, depression accompanying dementia, anxiety, psychosis, social affective disorders, and/or cognitive disorders), manic-depressive psychoses, bipolar disorders, extreme psychotic states (such as mania, schizophrenia, and excessive mood swings where behavioral stabilization is desired). The compounds and pharmaceutical compositions described herein can also be used alone or in combination therapy for the treatment or prevention of attention disorders such as ADHD (attention deficit hyperactivity disorders), autism, anxiety states, generalized anxiety, agoraphobia, as well as those behavioral states characterized by social withdrawal.

The compounds and pharmaceutical compositions described herein can be used alone or in combination therapy for the treatment or prevention of neurological or neurodegenerative disorders. Examples of neurodegenerative diseases include dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease, metabolism; toxins; anoxia and vitamin deficiency; and mild cognitive impairment associated with aging, particularly Age Associated Memory Impairment. Examples of neurological disorders include amyotrophic lateral sclerosis (ALS), multiple sclerosis, epilepsy, ischemia, traumatic head or brain injury, brain inflammation, eye injury, stroke and neuroinflammation.

The compounds and pharmaceutical compositions described herein can be used alone or in combination therapy for the treatment or prevention of ocular disorders including, for example, glaucoma (such as normal-tension glaucoma), glaucoma-associated intraocular pressure retinitis, retinopathies, uveitis, and acute injury to the eye tissue (e.g., conjunctivitis). Ocular disorders also include neurodegenerative diseases conditions of the retina and the optic nerve, for example, in patients presenting risk factors for glaucoma, such as high intraocular pressure, family history of glaucoma, glaucoma in the contralateral eye and high myopia.

Compounds and compositions of the invention are also useful for veterinary treatment of companion animals, exotic animals and farm animals, including, without limitation, dogs, cats, mice, rats, hamsters, gerbils, guinea pigs, rabbits, horses, pigs and cattle.

In another embodiment, the invention provides a method of increasing CB receptor activity in a biological sample, comprising contacting said biological sample with a compound or composition of the invention. Use of a CB receptor agonist in a biological sample is useful for a variety of purposes known to one of skill in the art. Examples of such purposes include, without limitation, biological assay's and biological specimen storage.

### Combination Therapies

The compounds and pharmaceutical compositions described herein can be used in combination therapy with one or more additional therapeutic agents. For combination treatment with more than one active agent, where the active agents are in separate dosage formulations, the active agents may be administered separately or in conjunction, In addition, the administration of one element may be prior to, concurrent to, or subsequent to the administration of the other agent.

When co-administered with other agents, e.g. when co-administered with another pain medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day

When “combination therapy” is employed, an effective amount can be achieved using a first amount of a compound of Formula I or a pharmaceutically acceptable salt, solvate (e.g., hydrate), co-crystal or pro-drug thereof and a second amount of an additional suitable therapeutic agent (e.g., an agent to treat pain).

In one embodiment of this invention, the compound of Formula I and the additional therapeutic agent are each administered in an effective amount (i.e., each in an amount which would be therapeutically effective if administered alone). In another embodiment, the compound of Structural Formula I and the additional therapeutic agent are each administered in an amount which alone does not provide a therapeutic effect (a sub-therapeutic dose). In yet another embodiment, the compound of Structural Formula I can be administered in an effective amount, while the additional therapeutic agent is administered in a sub-therapeutic dose. In still another embodiment, the compound of Structural Formula I can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When co-administration involves the separate administration of the first amount of a compound of Structural Formula I and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of Formula I and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks prior to), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks subsequent to) the administration of a second therapy (e.g., a prophylactic or therapeutic agent such as an anti-cancer agent) to a subject.

Additional therapeutic agents include, without limitation:

pain-relieving agents such as acetaminophen or paracetamol;

non-steroidal anti-inflammatory drugs (NSAIDs) such as propionic acid derivatives (alminoprofen, benoxaprofen, bucloxic acid, carprofen, fenhufen, fenoprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (meclofenamic acid, mefe-namic acid, and tolfenamic acid), biphenyl-carboxylic acid derivatives, oxicams (isoxicam, meloxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and COX-2 inhibitors, such as the coxibs (celecoxib, deracoxib, valdecoxib, rofecoxib, parecoxib and etoricoxib);

other pain relieving agents such as gabapentin, topical capsaicin, tanezumab, esreboxetine;

cannabinoid-receptor agonists such as Dronabinol, Δ9-THC, CP-55940, WIN-55212-2, HU-210;

opiate-receptor agonists such as morphine, propoxyphene (Darvon), tramadol, buprenorphin;

sodium-channel blockers such as carbamazepine, mexiletine, lamotrigine, pregabaline, tectin, NW-1029, CGX-1002;

N-type calcium-channel blockers such as Ziconotide, NMED-160, SPI-860; serotonergic and noradrenergic modulators such as SR-57746, paroxetine, duloxetine, clonidine, amitriptyline, citalopram;

local anesthetics such as ambroxol, lidocaine;

VR1 agonists and antagonists such as NGX-4010, WL-1002, ALGRX-4975, WL-10001, AMG-517;

agents used for migraine, such as sumatriptan, zolmitriptan, naratriptan, eletriptan, rauwolscine, yohimbine, metoclopramide;

anti-inflammatory and/or immunosuppressive agents such as methotrexate, cyclosporin A (including, for example, cyclosporin microemulsion), tacrolimus, corticosteroids, statins, interferon beta, Remicade (Infliximab), Enbrel (Etanercept) and Humira (Adalimumab);

agents designed to treat tobacco abuse (e.g., nicotine-receptor partial agonists, bupropion hypochloride (also known under the tradename Zyban™) and nicotine replacement therapies);

ADD/ADHD agents (e.g., Ritalin™ (methylphenidate hydrochloride), Strattera™ (atomoxetine hydrochloride), Concerta™ (methylphenidate hydrochloride) and Adderall™ (amphetamine aspartate; amphetamine sulfate; dextroamphetamine saccharate; and dextroamphetamine sulfate);

agents to treat alcoholism, such as opioid antagonists (e.g., naltrexone (also known under the tradename ReVia M) and nalmefene), disulfuram (also known under the tradename Antabuse™), and acamprosate (also known under the tradename Campral™);

agents for reducing alcohol withdrawal symptoms such as benzodiazepines, beta-blockers, clonidine, carbamazepine, pregabalin, and gabapentin (Neurontin™);

antihypertensive agents such as ACE inhibitors and Angiotensin II Receptor blockers such as benazepril, captopril, enalapril, fosinopril, lisinopril, candesartan, eprosartan, Irbesartan, losartan, olmesartan, telmisartan, valsartan, Renin inhibitors such as aliskiren, vasodilators such as minoxidil;

agents used to treat glaucoma such as direct-acting Miotics (cholinergic agonists), indirect-acting Miotics (cholinesterase inhibitors), Carbonic anhydrase inhibitors (e.g., Acetazolamide, Methazolamide, Brinzolamide, Dorzolamide, Selective adrenergic agonists (e.g., Apraclonidine, Brimonidine), Beta-blockers (Timolol, Betaxolol, Carteolol, Levobetaxolol, Levobunolol, Metipranolol), Osmotic diuretics (e.g., Glycerin, Mannitol);

antidepressants, such as SSRIs (e.g., fluoxetine, citalopram, femoxetine, fluvoxamine, paroxetine, indalpine, sertraline, zimeldine), tricyclic antidepressants (e.g., imipramine, amitriptiline, chlomipramine and nortriptiline), dopaminergic antidepressants (e.g., bupropion and amineptine), SNRIs (e.g., venlafaxine and reboxetine);

cognitive improvement agents (e.g., donepezil hydrochloride (Aircept™) and other acetylcholinesterase inhibitors);

anti-emetic agents (e.g., 5HT3 antagonists) such as ondansetron, granisetron, metoclopramide;

neuroprotective agents such as memantine, L-dopa, bromocriptine, pergolide, talipexol, pramipexol, cabergoline, neuroprotective agents currently under investigation including anti-apoptotic drugs (CEP 1347 and CTCT346), lazaroids, bioenergetics, antiglutamatergic agents and dopamine receptors. Other clinically evaluated neuroprotective agents are the monoamine oxidase B inhibitors selegiline and rasagiline, dopamine agonists, and the complex I mitochondrial fortifier coenzyme Q10;

antipsychotic medications (e.g., ziprasidone (Geodon™), risperidone (Risperdal™), and olanzapine (Zyprexa™);

agents used for multiple sclerosis such as beta-interferon (e.g., Avonex™, Betaseron™) and Copaxone.

disease-modifying antirheumatic drugs (DMARDS) such as methotrexate, azathioptrine, leflunomide, pencillinamine, gold salts, mycophenolate mofetil, cyclophosphamide; biological response modifiers (BRMs) such as Enbrel, Remicade, IL-1 antagonists; NSAIDS such as piroxicam, naproxen, indomethacin, ibuprofen and the like; COX-2-selective inhibitors such as Celebrex™; COX-1 inhibitors such as Feldene; immunosuppressives such as steroids, cyclosporine, Tacrolimus, rapamycin and the like;

PDE4 inhibitors such as theophylline, drotaverine hydrochloride, cilomilast, roflumilast, denbufylline, rolipram, tetomilast, enprofylline, arofylline, cipamfylline, tofimilast, filaminast, piclamilast, (R)-(+)-4-[2-(3-cyclopentyloxy-4-methoxyphenyl)-2-phenylethyl]pyridine, mesopram, N-(3,5-dichloro-4-pyridinyl)-2-[1-(4-fluorobenzyl)-5-hydroxy-1H-indol-3-yl]-2-oxoacetamide, CDC-801 (Celgene), CC-1088 (Celgene), Lirimilast, ONO-6126 (Ono), CC-10004 (Celgene) and MN-001 (Kyorin), ibudilast and pentoxifylline, for use in treating inflammation, lung disorders and as bronchodilators;

corticosteroids such as betamethasone, budesonide, cortisone, dexamethasone, hydrocortisone, methylprednisolone, prednisolone, prednisone and triamcinolone;

histamine H1-receptor antagonists such as bromopheniramine, chlorpheniramine, dexchlorpheniramine, triprolidine, clemastine, diphenhydramine, diphenylpyraline, tripelennamine, hydroxyzine, methdiazine, promethazine, trimeprazine, azatadine, cyproheptadine, antazoline, pheniramine pyrilamine, astemizole, terfenadine, loratadine, cetirizine, desloratadine, fexofenadine and levocetirizine;

histamine H2-receptor antagonists such as cimetidine, famotidine and ranitidine;

proton-pump inhibitors such as omeprazole, pantoprazole and esomeprazole;

leukotriene antagonists and 5-lipoxygenase inhibitors such as zafirlukast, montelukast, pranlukast and zileuton;

nicotinic acetylcholine receptor agonists such as ABT-202, A-366833, ABT-594; BTG-102, A-85380, CGX1204;

P2×3-receptor antagonists such as A-317491, ISIS-13920, AZD-9056;

NGF agonists and antagonists such as RI-724, RI-1024, AMG-819, AMG-403, PPH 207;

NK1 and NK2 antagonists such as DA-5018, R-116301; CP-728663, ZD-2249;

NMDA antagonist such as NER-MD-11, CNS-5161, EAA-090, AZ-756, CNP-3381; potassium-channel modulators such as CL-888, ICA-69673, retigabine;

GABA modulators such as lacosamide; and

serotonergic and noradrenergic modulators such as SR-57746, paroxetine, duloxetine, clonidine, amitriptyline, citalopram, flibanserin.

### Methods of Preparing the Compounds

The compounds of Formula I may be prepared according to the schemes and examples depicted and described below. Unless otherwise specified, the starting materials and various intermediates may be obtained from commercial sources, prepared from commercially available compounds or prepared using well-known synthetic methods.

### Syntheses

General synthetic procedures for the compounds of this invention are described below. The synthetic schemes are presented as examples and do not limit the scope of the invention in any way.

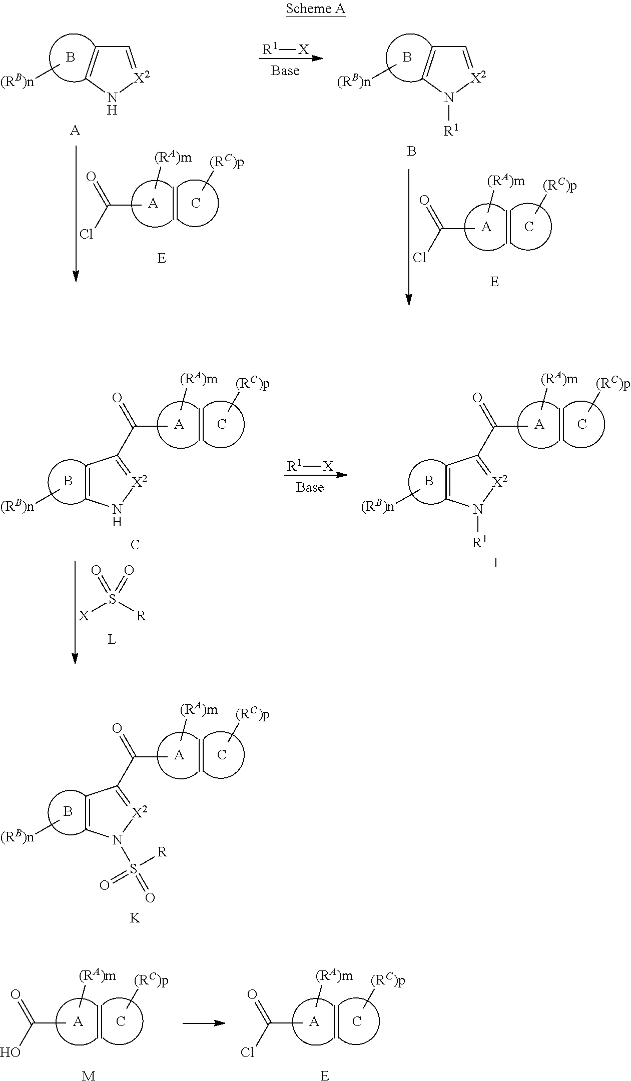

Generally, the compounds of the invention can be prepared from a compound of Formula A by acylation with a compound of Formula E and alkylation by R¹—X, wherein R¹ is defined herein and X is a leaving group. An acid chloride of Formula E can be made by converting the corresponding carboxylic acid of Formula M using a reagent such as thionyl chloride, oxalyl chloride or PCl₅. The alkylation and acylation step may be performed in either order, as shown in Scheme A. Alkylation is typically performed by treating a compound of Formula A or C with a strong base, such as sodium hydride, sodium hydroxide, or sodium carbonate, in the presence of, or followed by the addition of R¹—X. The acylation step can be performed by treating a compound of Formula A or B with a compound of Formula E under appropriate conditions. An example of conditions appropriate for acylation is Friedel-Craft's conditions, wherein the acylation step is performed using an aluminum activating agent, such as diethylaluminum chloride or aluminum trichloride, in a non-polar solvent.

Alternatively, a sulfone of Formula K can be synthesized from a compound of Formula C. This can be accomplished by treating a compound of Formula C with a base, such as sodium hydride, followed by an appropriate sulfonyl compound of formula L, wherein X is a leaving group, preferably chloride.

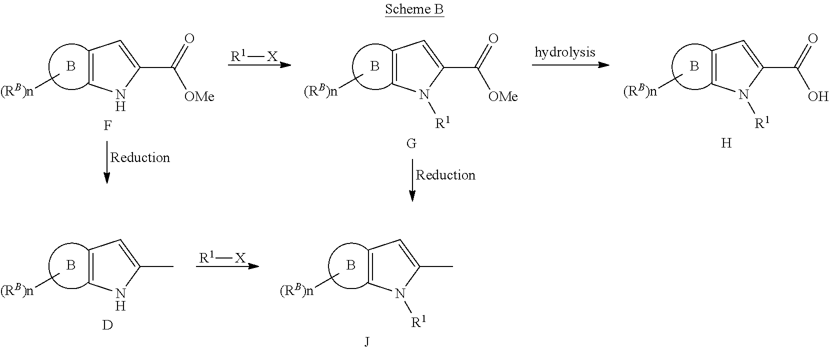

Compounds of Formula A and B, wherein X² is C-Me are represented in Scheme B by Formulas D and J respectively. Compounds of Formula J can be synthesized by alkylating a compound of Formula F with an alkylation agent represented in Scheme B by R¹—X followed by reduction of the methyl ester group. The alkylation and reduction steps can be performed in either order, wherein the alkylation step is described above. The reduction of the ester moiety can be accomplished using a suitable reducing reagent, such as lithium aluminum hydride.

Alternatively, compounds of Formula A, wherein X² is a carboxyl group are represented in Scheme B as Formula H, and can be synthesized from a compound of Formula

F. A carboxylic acid of Formula H can be synthesized by hydrolysis of the ester moiety of Formula G, which is the product of the alkylation of a compound of Formula F.

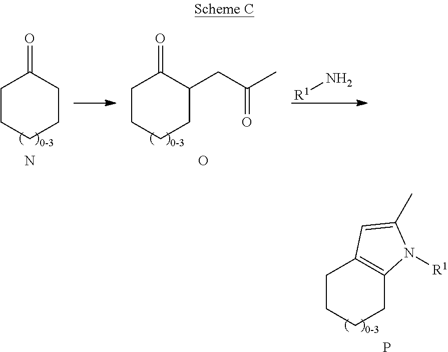

Compounds of Formula A, wherein Ring B is a 5-8-membered carbocyclic ring are represented by Formula P in Scheme C. A compound of Formula 0 can be synthesized in a two-step reaction, wherein a ketone of Formula N is treated with allyl alcohol, 2,2-dimethoxypropane and p-toluenesulfonic acid in the first step, and molecular oxygen, diacetoxycopper hydrate and palladium(II) chloride in the second step. A compound of Formula P is then produced from Formula 0 via a Paal-Knorr pyrrole synthesis using R¹—NH₂.

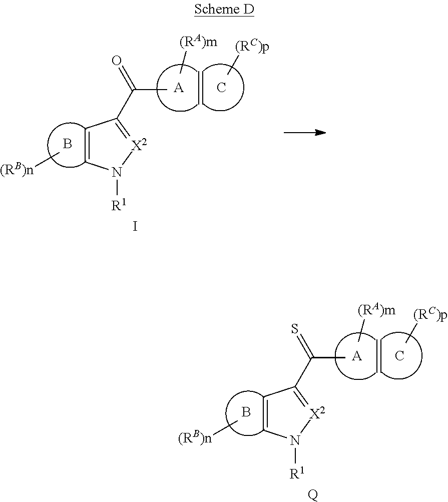

The ketone moieties of compounds of Formula I can also be converted to the corresponding thiocarbonyls using a reagents such as Lawesson's Reagent. Furthermore, if Ring A or Ring C is contains a substitutable nitrogen atom, such as in a pyrrole ring, then that nitrogen can be substituted analogously to the addition of the R¹ reagent shown above.

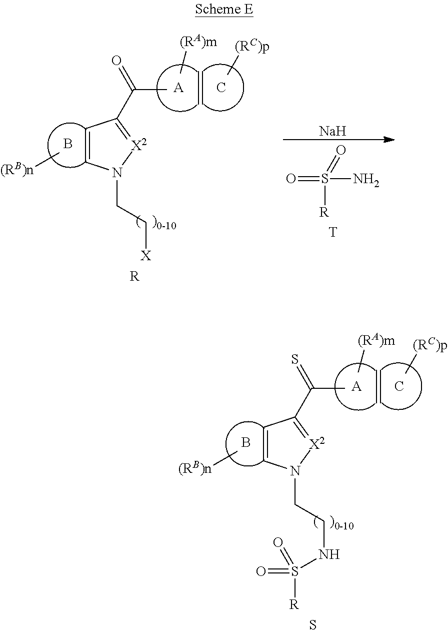

Compounds of Formula I, wherein R¹ is an alkyl chain substituted by a leaving group, X, are represented by Formula R, wherein X is preferably Cl, Br, I, p-toluenesulfonate, trifluoromethylsulfonate or methylsulfonate. Sulfonamides of Formula S can be synthesized from compounds of Formula R by treatment with a base, such as sodium hydride, and a sulfonamide of Formula T.

### EXAMPLES

All references provided in the Examples are herein incorporated by reference. As used herein, all abbreviations, symbols and conventions are consistent with those used in the contemporary scientific literature. See, e.g., Janet S. Dodd, ed., The ACS Style Guide: A Manual for Authors and Editors, 2^{nd} Ed., Washington, D.C.: American Chemical Society, 1997, herein incorporated in its entirety by reference.

### General Analytical Techniques Used

LC/MS was run on a Waters Acquity system, using a Polar C 18 column and a solvent gradient of 5 to 60% acetonitrile/water over 5 min with an ionization method using electrospray.

Automated column chromatography was run using an ISCO system (models used were Companion, Combiflash or Combiflash Rf).

Microwave reactions were run on a Personal Chemistry Optimizer system with the following conditions: temperature 0-240° C.; pressure 0-21 bar; power 0-300 W

Purification by HPLC was carried out using a Varian Prepstar system using Solvent A 0.1% Trifluoroacetic acid in water and Solvent B Trifluoroacetic acid in acetonitrile with the time gradients described below.

| Time | Solvent A | Solvent B | Flow | Inject wait |
|---|---|---|---|---|
| 0:00 | 90 | 10 | 15 | x |
| 35:00 | 5 | 95 | 15 | |
| 43:00 | 5 | 95 | 15 | |
| 45:00 | 95 | 5 | 15 | |
| 50:00 | 98 | 2 | 0 | |

### Example 1

### Alkylation of Bicyclic Ring System Pyrrole Nitrogen (Method A)

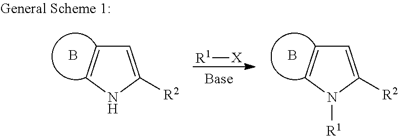

Preparation of Intermediate i

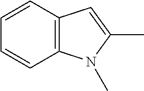

To a solution of 2-methyl-1H-indole (1 g, 7.6 mmol) in DMF (300 mL), at −30° C., was added NaH (60% in mineral oil, 365 mg, 9.12 mmol). The reaction mixture was stirred at −30° C. for 50 min, followed by the addition of iodomethane (0.515 mL, 8.36 mmol). The reaction mixture was stirred at −30° C. for 30 min and slowly allowed to warm up to room temperature. The reaction mixture was then stirred at room temperature overnight. The reaction was quenched by adding water (400 mL) and the mixture was extracted with DCM (3×100 mL). The organic layers were combined, washed with brine, dried over MgSO₄, filtered and concentrated under reduced pressure. The crude product was purified by silica gel flash chromatography (20-70% EtOAc in Hexanes) to give 1,2-dimethyl-1H-indole (529.9 mg, 48%). ¹H NMR (CDCl₃/400 MHz) δ 7.50 (d, 1H), 7.22 (d, 1H), 7.15-7.11 (m, 1H), 7.07-7.05 (m, 1H), 6.23 (s, 1H), 3.60 (s, 3H), 2.39 (s, 3H).

### Example 2

### Introduction of Methyl at Position 2 of Bicyclic Ring System

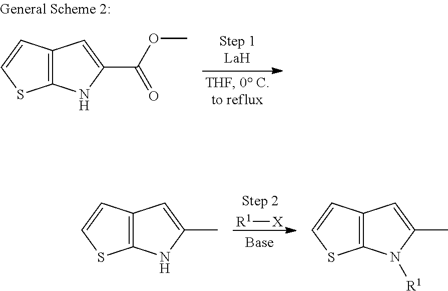

Preparation of Intermediate ii:

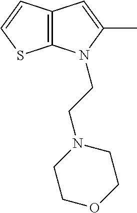 [SMILES: Cc1cc2ccsc2n1CCN1CCOCC1]

### Step 1

A 2.0 M solution of lithium aluminum hydride (LAH; 308 ml, 616 mmol) in THF was slowly added to a 0° C. solution of methyl 6H-thieno[2,3-b]pyrrole-5-carboxylate (40.0 g, 221 mmol) in 400 mL of THF. A significant amount of hydrogen gas evolved. The mixture was heated at reflux for 5 h. The reaction mixture was then cooled to 0° C. and quenched by the slow addition of water (23 mL), followed by a 15% NaOH solution (23 mL) and water (70 mL). The mixture was stirred vigorously throughout the quenching process. The precipitate generated was filtered off and discarded. The filtrate was diluted with Et₂O (200 mL) and washed with H₂O (50 mL). The organic layer was dried over MgSO₄ and concentrated under reduced pressure. It was then azeotroped twice with benzene to afford 5-methyl-6H-thieno[2,3-b]pyrrole as a tan solid (25.1 g, 83%). ¹H NMR (CDCl₃/400 MHz) δ 7.95 (br s, 1H), 6.91 (dd, 1H), 6.77 (d, 1H), 6.13 (dd, 1H), 2.39 (d, 3H).

The product obtained in Step 1 was used directly in Step 2 without further purification.

### Step 2

Potassium hydroxide (4.09 g, 72.9 mmol) and H₂O (110 μL, about 10 drops) were added to a solution of 4-(2-chloroethyl)morpholine hydrochloride (3.26 g, 18.50 mmol) and 5-methyl-6H-thieno[2,3-b]pyrrole (2.54 g, 18.50 mmol) in 31 mL of DMSO. The reaction mixture was heated at 75° C. with stirring for 1.5 h, during which time the reaction mixture darkened to a deep brown. The reaction mixture was diluted with H₂O and extracted with EtOAc (3×). The combined organic layers were washed with H₂O (2×) and the combined organic layers were dried and the solvent evaporated. The residue was purified by flash silica gel chromatography (10%-70% EtOAc in hexanes). The fractions were collected and concentrated under reduced pressure. The crude product was azeotroped twice with benzene to afford 4-(2-(5-methyl-6H-thieno[2,3-b]pyrrol-6-yl)ethyl)morpholine as a light yellow solid (2.95 g, 81%, small crystals). ¹H NMR (CDCl₃/400 MHz) δ 6.92 (d, 1H), 6.76 (d, 1H), 6.12 (d, 1H), 4.04 (d, 2H), 3.71 (t, 4H), 2.71 (t, 2H), 2.49 (t, 4H), 2.37 (d, 3H); MS m/z: 251.14 (M+1).

### Example 3

### Cycloalkylpyrroles

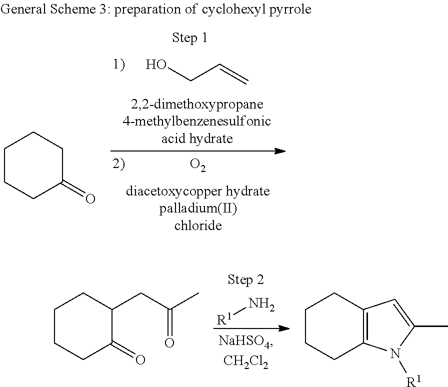

Preparation of Intermediate iii:

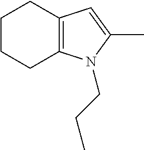

### Step 1

In a 250 ml round bottom flask equipped with a short-path distillation flask, the mixture of cyclohexanone (15.84 mL, 153 mmol), allyl alcohol (31.2 mL, 459 mmol), 2,2-dimethoxypropane (37.5 mL, 306 mmol), 4-methylbenzenesulfonic acid hydrate (0.029 g, 0.153 mmol), and benzene (102 mL) was heated at 130° C. An azetropic mixture of benzene, methanol, and acetone was removed. After the distillation had completed, the mixture was heated at 190° C. The mixture was stirred for an additional 30 min. It was then additionally purified by vacuum distillation (40° C., 1 torr) to give 2-allylcyclohexanone (11.16 g, 81 mmol, 52.8% yield) as a clear oil. ¹H NMR (CDCl₃/400 MHz) δ 5.78-5.76 (m, 1H), 5.04-4.98 (m, 2H), 2.55-2.52 (m, 1H), 2.38-2.24 (m, 3H), 2.15-1.96 (m, 3H), 1.87-1.85 (m, 1H), 1.69-1.63 (m, 3H), 1.38-1.34 (m, 1H).

### Step 2

A 2-necked 500 ml round bottom flask equipped with a stir bar and septa was charged with palladium(II) chloride (0.178 g, 1.006 mmol), diacetoxycopper hydrate (0.803 g, 4.02 mmol), DMA (63.9 ml), and water (3.19 ml). The suspension was stirred under a positive pressure of oxygen (provided by 3 oxygen-filled balloons) at 80° C. for 4 h. The reaction mixture immediately became dark brown and dark residues coated the inside of the flask. Added 2-allylcyclohexanone (5 ml, 33.5 mmol) and a fourth balloon filled with oxygen. The reaction mixture was then stirred at 80° C. overnight. The reaction was 70% complete as determined by LC/MS. The mixture was extracted with a 1:1 EtOAc:H₂O mixture (100 ml×5). The organic layers were combined and washed with water (5×50 ml). The combined organic layers were dried over MgSO₄, filtered, and concentrated under reduced pressure to give an oil. The crude oil was purified by chromatography on silica gel using a Biotage system to give 2-(2-oxopropyl)cyclohexanone (2.62 g, 16.99 mmol, 50.7% yield). ¹H NMR (CDCl₃/400 MHz) δ 3.00-2.94 (m, 2H), 2.38-2.34 (m, 2H), 2.19 (s, 3H), 2.15-2.07 (m, 3H), 1.87-1.83 (m, 1H), 1.74-1.59 (m, 2H), 1.37-1.33 (m, 1H).

### Example 4

### Alkylation of Bicyclic Ring System Pyrrole Nitrogen (Method B)

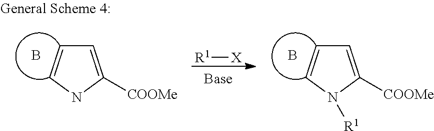

N-Alkylation of Thienopyrrole iv

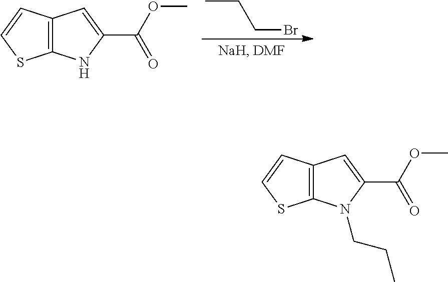

To a solution of 2-methyl 6H-thieno[2,3-b]pyrrole-5-carboxylate (5 g, 27.6 mmol) in DMF (50 mL), at 0° C., was added NaH (60%, 795 mg, 33 mmol). The reaction mixture was stirred at 0° C. for 50 min, followed by the addition of 1-bromopropane (2.7 mL, 30.3 mmol). The resulting mixture was stirred at 0° C. for 30 min and then slowly allowed to warm up to room temperature. The reaction mixture was stirred at this temperature overnight and then was quenched by adding water (200 mL). The mixture was extracted with DCM (3×150 mL). The organic layers were combined, washed with brine, dried over MgSO₄, filtered and concentrated under reduced pressure. The crude product was purified by silica gel flash chromatography (20-70% EtOAc in Hexanes) to give methyl 6-propyl-6H-thieno[2,3-b]pyrrole-5-carboxylate (4.1 g, 66%). ¹H NMR (CDCl₃/400 MHz) δ 7.15 (s, 1H), 6.98 (d, 1H), 6.90 (d, 1H), 4.40 (t, 2H), 3.85 (s, 3H), 1.89 (m, 2H), 0.94 (t, 3H).

### Example 5

### Ester Saponification

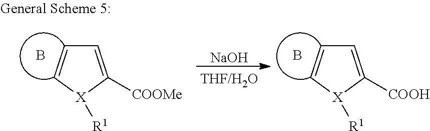

Saponification of Ester v:

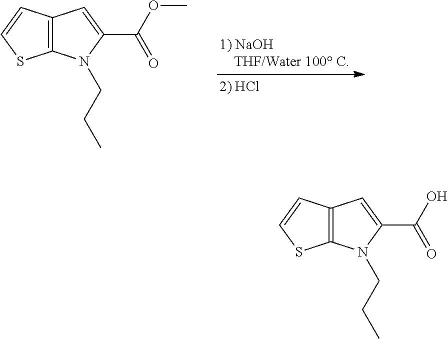

In a 250 ml round bottom flask equipped with a stir bar and a reflux condenser, a mixture of methyl 6-propyl-6H-thieno[2,3-b]pyrrole-5-carboxylate (5 g, 22.4 mmol) and sodium hydroxide (1.79 g, 44.8 mmol) in 1:1 THF/water (200 mL) was heated at reflux. The reaction was complete after heating for 1 h as determined by LC/MS. The mixture was then poured over ice and acidified slowly with 1N HCl (to ca. pH=4). Once the ice had fully melted, the precipitate formed was filtered to provide 6-propyl-6H-thieno[2,3-b]pyrrole-5-carboxylic acid (4.62 g, quantitative). ¹H NMR (CDCl₃/400 MHz) δ 7.29 (s, 1H), 7.00 (d, 1H), 6.93 (d, 1H), 4.42 (t, 2H), 1.91 (q, 2H), 0.95 (t, 3H);

### Example 6

### Formation of Acid Chloride for Coupling

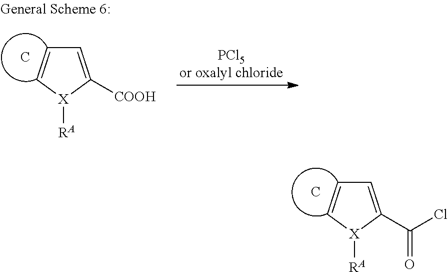

Preparation of Acid Chloride of Compound vi:

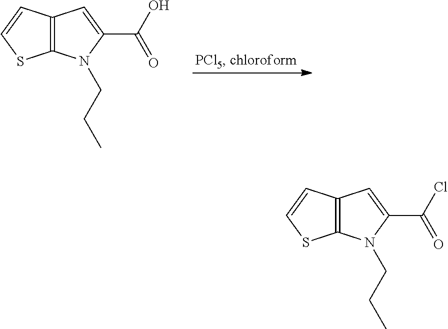

To a mixture of 6-propyl-6H-thieno[2,3-b]pyrrole-5-carboxylic acid (700 mg, 3.35 mmol) in chloroform (5 mL), at room temperature, was added phosphorous pentachloride (731 mg, 3.51 mmol). The reaction mixture was then heated at 50° C. for 4 h. All solvent was removed under reduced pressure and the crude product was dried under reduced pressure and used in next step without further purification. Complete coversion was assumed. ¹H NMR (CDCl₃/400 MHz) δ 7.49 (s, 1H), 7.00 (d, 1H), 6.97 (d, 1H), 4.30 (t, 2H), 1.86 (m, 2H), 0.93 (t, 3H).

Preparation of Acid Chloride of Compound vii:

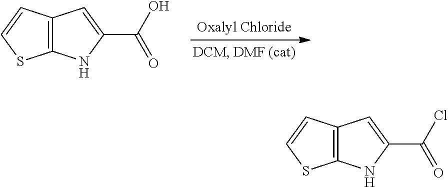 [SMILES: O=C(O)c1cc2ccsc2[nH]1 ; O=C(Cl)c1cc2ccsc2[nH]1]

To a solution of 6-ethyl-6H-thieno[2,3-b]pyrrole-5-carboxylic acid (1.447 g, 8.7 mmol) in DCM (35 mL) at room temperature was added oxalyl chloride (1.10 g, 8.7 mmol), followed by 3 drops of N,N-dimethylformamide (catalytic amount). Gas evolved and the resulting brown solution was stirred at room temperature for 17 h. It was then concentrated to yield a dark brown oil. This was then azeotroped once with toluene and used in the next reaction without further purification. Complete conversion was assumed. ¹H NMR (CDCl₃/400 MHz) δ 7.31 (d, 1H), 6.96 (d, 1H), 6.92 (d, 1H).

Preparation of Acid Chloride of Compound viii:

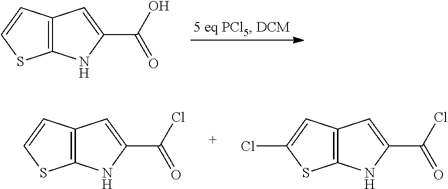 [SMILES: O=C(O)c1cc2ccsc2[nH]1 ; O=C(Cl)c1cc2ccsc2[nH]1 ; O=C(Cl)c1cc2cc(Cl)sc2[nH]1]

To a solution of 6-ethyl-6H-thieno[2,3-b]pyrrole-5-carboxylic acid (1.4 g, 8.4 mmol) in DCM (35 mL) at room temperature was added phosphorous pentachloride (8.7 g, 0.54 mmol), followed by 3 drops of N,N-dimethylformamide (catalytic amount). Gas evolved and the brown solution (reaction darkened over time) was stirred at room temperature for 17 h. It was then concentrated to yield a dark brown oil that was then azeotroped once with toluene. This was used in the next reaction without further purification. By LC/MS, the ratio of 6H-thieno[2,3-b]pyrrole-5-carbonyl chloride and 2-chloro-6H-thieno[2,3-b]pyrrole-5-carbonyl chloride was determined to be about 3:1. Product was used directly in next step without additional purification.

### Example 7

### Coupling of the Two Bicyclic Ring Systems

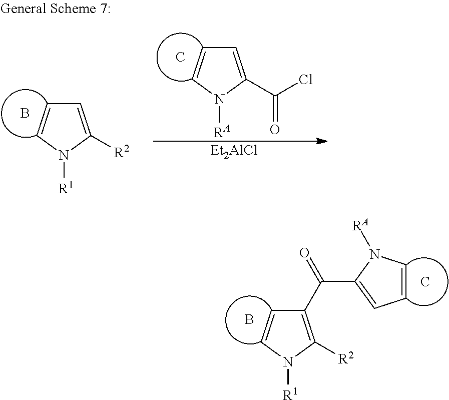

Preparation of Compound ix

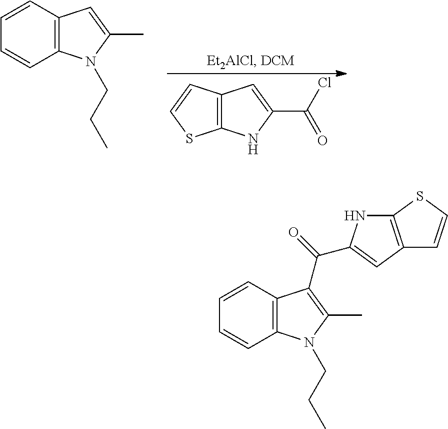 [SMILES: CCCn1c(C)cc2ccccc21 ; O=C(Cl)c1cc2ccsc2[nH]1 ; CCCn1c(C)c(C(=O)c2cc3ccsc3[nH]2)c2ccccc21]

To a solution of 2-methyl-1-propyl-1H-indole (148 mg, 0.46 mmol) in DCM (5 mL), at 0° C., was added diethylaluminum chloride (0.69 mL, 0.69 mmol). The reaction mixture turned dark orange color and was stirred for 5 min at this temperature. To the mixture was then added a solution of 6H-thieno[2,3-b]pyrrole-5-carbonyl chloride (85.0 mg, 0.46 mmol) in DCM (5 mL). The reaction mixture immediately turned to dark brown. The reaction mixture was stirred for 1 h at 0° C., followed by 4 h at room temperature. The reaction was then quenched by adding water (100 mL). The mixture was extracted with DCM (3×50 mL) and the organic layers were combined, washed with brine, dried over MgSO₄, filtered and concentrated under reduced pressure. The crude product was further purified by flash chromatography (20-70% EtOAc in Hexanes) to afford (2-methyl-1-propyl-1H-indol-3-yl)(6H-thieno[2,3-b]pyrrol-5-yl)methanone (16 mg, 11%). ¹H NMR (CDCl₃/400 MHz) δ 10.04 (br s, 1H), 7.89 (d, 1H), 7.33 (d, 1H), 7.23-7.19 (m, 1H), 7.15-7.11 (m, 1H), 7.03 (d, 1H), 6.96 (dd, 1H), 6.89 (d, 1H), 4.12 (t, 2H), 2.69 (s, 3H), 1.85 (q, 2H), 1.01 (t, 3H).

The following compounds were prepared according to Example 7 and General Scheme 7:

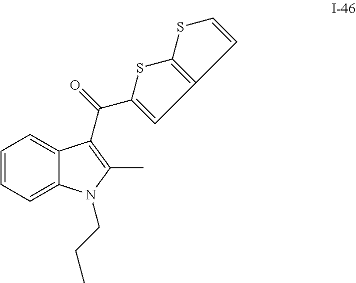 [SMILES: CCCn1c(C)c(C(=O)c2cc3ccsc3s2)c2ccccc21]

¹H NMR (CDCl₃/400 MHz) δ 7.76 (s, 1H), 7.70 (d, 1H), 7.38 (d, 1H), 7.34 (d, 1H), 7.23-7.19 (m, 2H), 7.13-7.09 (m, 1H), 4.12 (t, 2H), 2.64 (s, 3H), 1.85 (q, 2H), 1.01 (t, 3H).

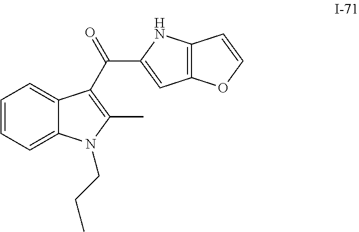 [SMILES: CCCn1c(C)c(C(=O)c2cc3occc3[nH]2)c2ccccc21]

¹H NMR (CDCl₃/400 MHz) δ 9.22 (s, 1H), 7.84 (d, 1H), 7.53 (d, 1H), 7.31 (d, 1H), 7.19 (td, 1H), 7.11 (td, 1H), 6.72 (q, 1H), 6.50 (dd, 1H), 4.10 (t, 2H), 2.64 (s, 3H), 1.84 (q, J=7.6 Hz, 2H), 1.00 (t, 3H).

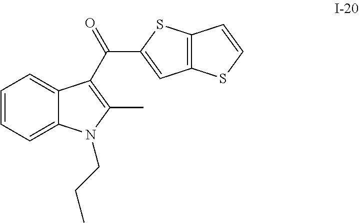 [SMILES: CCCn1c(C)c(C(=O)c2cc3sccc3s2)c2ccccc21]

¹H NMR (CDCl₃/400 MHz) 7.80 (d, 1H), 7.68 (d, 1H), 7.67 (t, 1H), 7.34-7.32 (m, 2H), 7.23-7.19 (m, 1H), 7.13-7.09 (m, 1H), 4.12 (t, 2H), 2.64 (s, 3H), 1.85 (q, 2H), 1.01 (t, 3H).

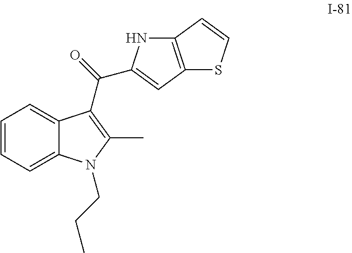 [SMILES: CCCn1c(C)c(C(=O)c2cc3sccc3[nH]2)c2ccccc21]

¹H NMR (CDCl₃/400 MHz) δ 9.55 (br s, 1H), 7.85 (d, 1H), 7.34 (d, 1H), 7.31 (s, 1H), 7.20 (td, 1H), 7.12 (td, 1H), 7.06 (d, 1H), 7.00 (dd, 1H), 4.11 (t, 2H), 2.65 (s, 3H), 1.85 (q, 2H), 1.00 (t, 3H).

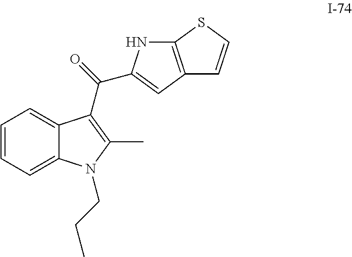 [SMILES: CCCn1c(C)c(C(=O)c2cc3ccsc3[nH]2)c2ccccc21]

¹H NMR (CDCl₃/400 MHz) δ 10.34 (br s, 1H), 7.90 (d, 1H), 7.33 (d, 1H), 7.21 (td, 1H), 7.13 (td, 1H), 7.03 (d, 1H), 6.97 (dd, 1H), 6.90 (d, 1H), 4.12 (t, 2H), 2.69 (s, 3H), 1.85 (q, 2H), 1.01 (t, 3H).

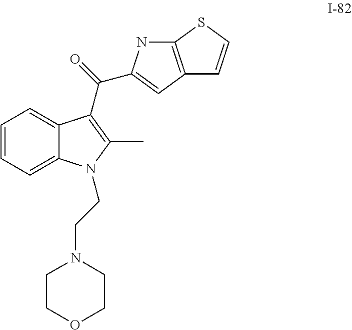 [SMILES: Cc1c(C(=O)c2cc3ccsc3[nH]2)c2ccccc2n1CCN1CCOCC1]

¹H NMR (CDCl₃/400 MHz) δ 10.42 (br s, 1H), 7.84 (d, 1H), 7.28 (d, 1H), 7.17 (td, 1H), 7.08 (td, 1H), 6.96 (d, 1H), 6.90 (d, 1H), 6.84 (d, 1H), 4.23 (t, 2H), 3.67 (d, 4H), 2.68-2.64 (m, 5H), 2.48 (t, 4H).

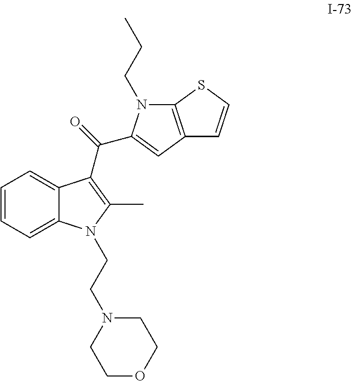 [SMILES: CCCn1c(C(=O)c2c(C)n(CCN3CCOCC3)c3ccccc23)cc2ccsc21]

¹H NMR (CDCl₃/400 MHz) δ 7.50 (d, 1H), 7.37 (d, 1H), 7.12 (t, 1H), 6.98 (t, 1H), 6.94 (d, 1H), 6.87 (d, 1H), 6.75 (s, 1H), 4.39 (t, 2H), 4.30 (t, 2H), 3.61 (t, 4H), 2.68 (t, 2H), 2.52 (s, 3H), 2.49 (br s, 4H), 1.86 (q, 2H) 0.85 (t, 3H).

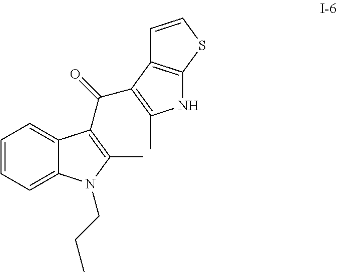 [SMILES: CCCn1c(C)c(C(=O)c2c(C)[nH]c3sccc23)c2ccccc21]

¹H NMR (CDCl₃/400 MHz) δ 8.51 (br s, 1H), 7.52 (d, 1H), 7.31 (d, 1H), 7.16 (td, 1H), 7.02 (td, 1H), 6.69 (t, 1H), 6.47 (d, 1H), 4.11 (t, 2H), 2.57 (s, 3H), 2.56 (s, 3H), 1.84 (q, 2H), 0.99 (t, 3H).

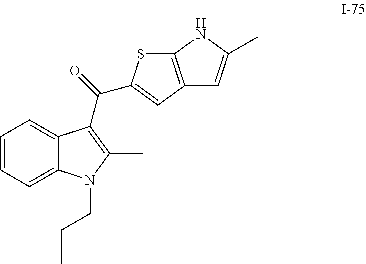 [SMILES: CCCn1c(C)c(C(=O)c2cc3cc(C)[nH]c3s2)c2ccccc21]

¹H NMR (CDCl₃/400 MHz) δ 8.35 (br s, 1H), 7.68 (d, 1H), 7.46 (s, 1H), 7.26 (d, 1H), 7.13 (td, 1H), 7.03 (t, 1H), 6.03 (d, 1H), 4.04 (t, 2H), 2.57 (s, 3H), 2.73 (s, 3H), 1.78 (q, 2H), 0.94 (t, 3H).

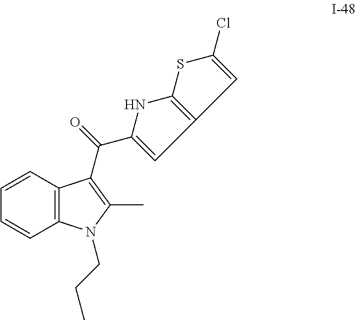 [SMILES: CCCn1c(C)c(C(=O)c2cc3cc(Cl)sc3[nH]2)c2ccccc21]

¹H NMR (MeOD/400 MHz) δ 7.68 (d, 1H), 7.45 (d, 1H), 7.22 (td, 1H), 7.11 (td, 1H), 6.97 (s, 1H), 6.89 (s, 1H), 4.24 (t, 2H), 2.63 (s, 3H), 1.87 (q, 2H), 1.03 (t, 3H).

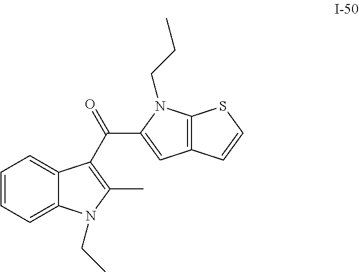 [SMILES: CCCn1c(C(=O)c2c(C)n(CC)c3ccccc23)cc2ccsc21]

¹H NMR (CDCl₃/400 MHz) δ 7.60 (d, 1H), 7.45 (d, 1H), 7.19 (t, 1H), 7.08-7.04 (m, 1H), 7.02 (d, 1H), 6.97 (d, 1H), 6.85 (s, 1H), 4.48 (t, 2H), 4.30 (t, 2H), 2.59 (s, 3H), 1.93 (q, 2H), 1.40 (t, 3H), 0.94 (t, 3H).

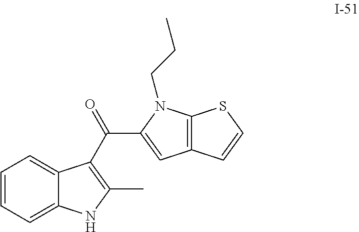 [SMILES: CCCn1c(C(=O)c2c(C)[nH]c3ccccc23)cc2ccsc21]

¹H NMR (CDCl₃/400 MHz) δ 7.61 (d, 1H), 7.34 (d, 1H), 7.11 (td, 1H), 7.04-7.00 (m, 2H), 6.97 (d, 1H), 6.88 (s, 1H), 4.46 (t, 2H), 2.55 (s, 3H), 1.92 (q, 2H), 0.93 (tq, 3H).

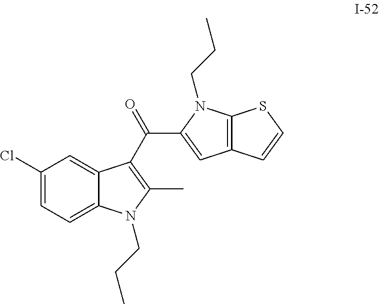 [SMILES: CCCn1c(C)c(C(=O)c2cc3ccsc3n2CCC)c2cc(Cl)ccc21]

¹H NMR (CDCl₃/400 MHz) δ 7.57 (d, 1H), 7.44 (d, 1H), 7.15 (dd, 1H), 7.04 (d, 1H), 6.99 (d, 1H), 6.83 (s, 1H), 4.48 (t, 2H), 4.20 (t, 2H), 2.57 (s, 3H), 1.94 (q, 2H), 1.84 (q, 2H), 1.02-0.93 (m, 6H).

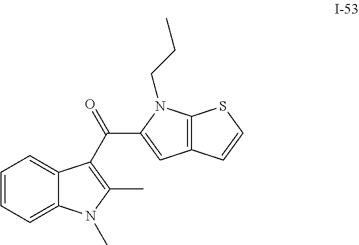 [SMILES: CCCn1c(C(=O)c2c(C)n(C)c3ccccc23)cc2ccsc21]

¹H NMR (CDCl₃/400 MHz) δ 7.60 (d, 1H), 7.44 (d, 1H), 7.20 (t, 1H), 7.07 (t, 1H), 7.02 (d, 1H), 6.97 (d, 1H), 6.85 (s, 1H), 4.78 (t, 2H), 3.79 (s, 3H), 2.58 (s, 3H), 1.93 (q, 2H), 0.94 (t, 3H).

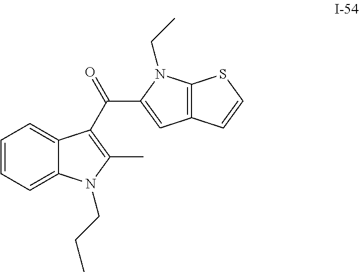 [SMILES: CCCn1c(C)c(C(=O)c2cc3ccsc3n2CC)c2ccccc21]

¹H NMR (CDCl₃/400 MHz) δ 7.60 (d, 1H), 7.44 (d, 1H), 7.18 (t, 1H), 7.08-7.05 (m, 1H), 7.03 (d, 1H), 6.97 (d, 1H), 6.83 (s, 1H), 4.52 (q, 2H), 4.21 (q, 2H), 2.58 (s, 3H), 1.86 (q, 2H), 1.49 (t, 3H), 1.00 (t, 3H).

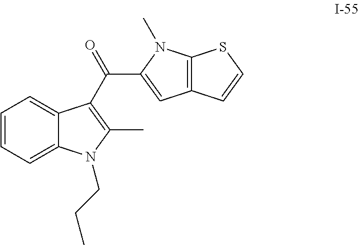 [SMILES: CCCn1c(C)c(C(=O)c2cc3ccsc3n2C)c2ccccc21]

¹H NMR (MeOD/400 MHz) δ 7.61 (d, 1H), 7.43 (d, 1H), 7.17 (td, 1H), 7.06 (d, 1H), 7.03 (d, 1H), 6.96 (d, 1H), 6.84 (s, 1H), 4.20 (t, 2H), 4.04 (s, 3H), 2.58 (s, 3H), 1.84 (q, 2H), 0.99 (t, 3H).

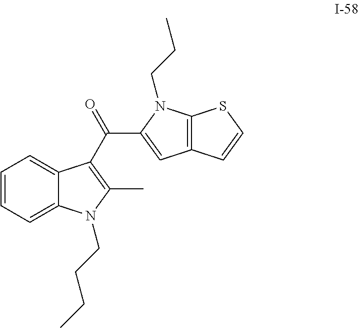 [SMILES: CCCCn1c(C)c(C(=O)c2cc3ccsc3n2CCC)c2ccccc21]

¹H NMR (CDCl₃/400 MHz) δ 7.59 (d, 1H), 7.42 (d, 1H), 7.18 (t, 1H), 7.05 (d, 1H), 7.01 (d, 1H), 6.95 (d, 1H), 6.83 (s, 1H), 4.46 (t, 2H), 4.22 (t, 2H), 2.57 (m, 3H), 1.92 (q, 2H), 1.80-1.73 (m, 2H), 1.46-1.42 (m, 2H), 1.00-0.91 (m, 6H).

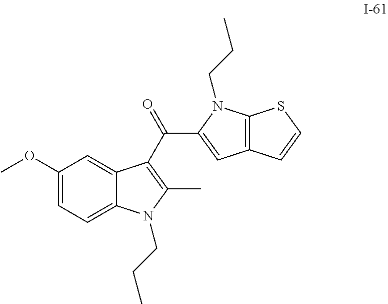 [SMILES: CCCn1c(C)c(C(=O)c2cc3ccsc3n2CCC)c2cc(OC)ccc21]

¹H NMR (CDCl₃/400 MHz) δ 7.32 (s, 1H), 7.05 (s, 1H), 6.90 (q, 2H), 6.71 (s, 1H), 6.21 (s, 1H), 4.55 (t, 2H), 3.98 (t, 2H), 3.81 (s, 3H), 2.43 (s, 3H), 2.00 (q, 2H), 1.75 (q, 2H), 1.02 (t, 3H), 0.91 (t, 3H).

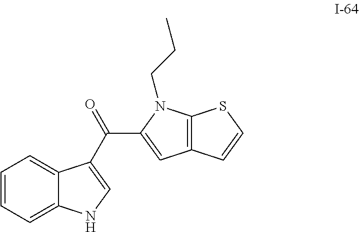 [SMILES: CCCn1c(C(=O)c2c[nH]c3ccccc23)cc2ccsc21]

¹H NMR (CDCl₃/400 MHz) δ 9.14 (br s, 1H), 8.38 (t, 1H), 7.75 (d, 1H), 7.40-7.38 (m, 1H), 7.29-7.25 (m, 2H), 7.00 (dd, 1H), 6.95 (d, 1H), 6.92 (dd, 2H), 4.46 (t, 2H), 1.94 (q, 2H), 0.93 (t, 3H).

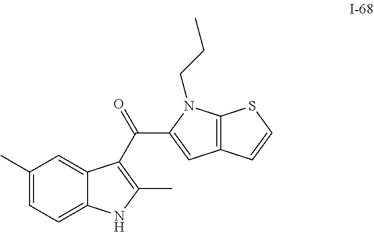 [SMILES: CCCn1c(C(=O)c2c(C)[nH]c3ccc(C)cc23)cc2ccsc21]

¹H NMR (CDCl₃/400 MHz) δ 8.41 (br s, 1H), 7.59 (s, 1H), 7.18 (d, 1H), 6.99-6.96 (m, 2H), 6.91 (s, 2H), 4.51 (t, 2H), 2.53 (s, 3H), 2.37 (s, 3H), 1.97 (q, 2H), 0.97 (t, 3H).

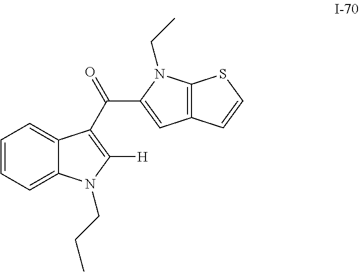 [SMILES: CCCn1cc(C(=O)c2cc3ccsc3n2CC)c2ccccc21]

¹H NMR (CDCl₃/400 MHz) δ 8.43-8.41 (m, 1H), 7.78 (s, 1H), 7.41-7.39 (m, 1H), 7.31 (q, 1H), 7.03 (d, 1H), 6.96 (s, 1H), 6.93 (d, 2H), 4.53 (q, 2H), 4.15 (t, 2H), 1.94 (q, 2H), 1.53 (q, 3H), 0.98 (t, 3H).

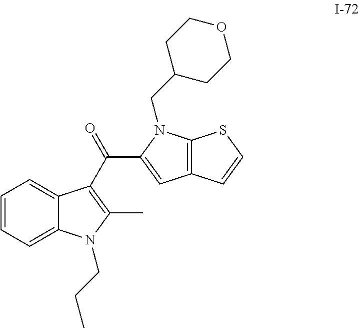 [SMILES: CCCn1c(C)c(C(=O)c2cc3ccsc3n2CC2CCOCC2)c2ccccc21]

¹H NMR (CDCl₃/400 MHz) δ 7.67 (d, 1H), 7.33 (d, 1H), 7.20 (d, 1H), 7.09 (q, 1H), 6.95-6.90 (m, 3H), 4.46 (d, 2H), 4.12 (d, 2H), 3.94 (dd, 2H), 3.33 (td, 2H), 2.63 (s, 3H), 2.31 (m, 1H), 1.86 (q, 2H), 1.58-1.45 (m, 4H), 1.02 (t, 3H).

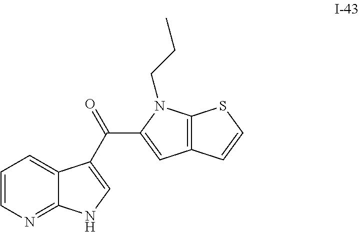 [SMILES: CCCn1c(C(=O)c2c[nH]c3ncccc23)cc2ccsc21]

¹H NMR (CDCl₃/400 MHz) δ 8.49 (dd, 1H), 7.96 (dd, 1H), 7.78 (t, 1H), 7.23 (dd, 1H), 7.01-6.98 (m, 2H), 6.59 (d, 1H), 4.45 (t, 2H), 2.03 (q, 2H), 1.02 (q, 3H).

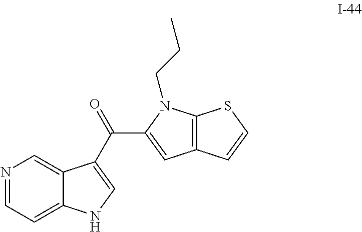 [SMILES: CCCn1c(C(=O)c2c[nH]c3ccncc23)cc2ccsc21]

¹H NMR (CDCl₃/400 MHz) δ 8.98 (d, 1H), 8.51 (d, 1H), 8.14 (d, 1H), 7.78 (d, 1H), 7.02 (dd, 2H), 6.73 (d, 1H), 4.43 (t, 2H), 1.98 (q, 2H), 0.99 (t, 3H).

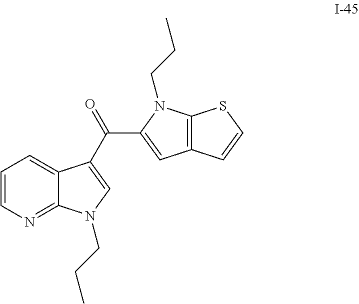 [SMILES: CCCn1cc(C(=O)c2cc3ccsc3n2CCC)c2cccnc21]

¹H NMR (CDCl₃/400 MHz) δ 8.63 (dd, 1H), 8.42 (dd, 1H), 7.90 (s, 1H), 7.25-7.24 (m, 1H), 7.04 (d, 1H), 7.00 (s, 1H), 6.95 (d, 1H), 4.46 (t, 2H), 4.33 (t, 2H), 2.00-1.94 (m, 4H), 1.01-0.85 (m, 6H).

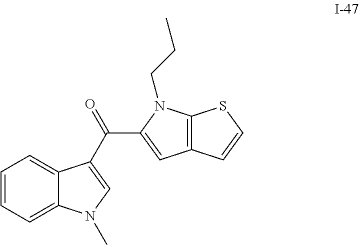 [SMILES: CCCn1c(C(=O)c2cn(C)c3ccccc23)cc2ccsc21]

¹H NMR (CDCl₃/400 MHz) δ 8.40 (d, 1H), 7.74 (s, 1H), 7.40-7.34 (m, 3H), 7.02 (d, 1H), 6.98 (s, 1H), 6.93 (d, 1H), 4.46 (t, 2H), 3.88 (s, 3H), 1.96 (q, 2H), 0.96 (t, 3H).

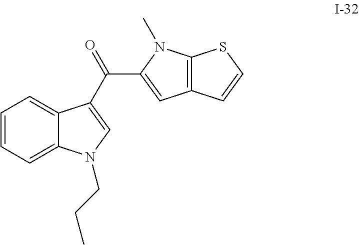 [SMILES: CCCn1cc(C(=O)c2cc3ccsc3n2C)c2ccccc21]

¹H NMR (CDCl₃/400 MHz) δ 8.42-8.40 (m, 1H), 7.79 (s, 1H), 7.42-7.40 (m, 1H), 7.34-7.31 (m, 2H), 7.04 (d, 1H), 6.97 (s 1H), 6.94 (d, 1H), 4.16 (t, 2H), 4.09 (s, 3H), 1.94 (q, 2H), 0.98 (t, 3H).

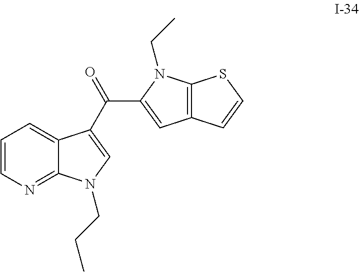 [SMILES: CCCn1cc(C(=O)c2cc3ccsc3n2CC)c2cccnc21]

¹H NMR (CDCl₃/400 MHz) δ 8.64 (dd, 1H), 8.42 (dd, 1H), 7.91 (s, 1H), 7.25-7.24 (m, 1H), 7.05 (d, 1H),), 7.00 (s, 1H), 6.96 (d, 1H), 4.53 (q, 2H), 4.33 (q, 2H), 1.97 (q, 2H), 1.54 (t, 3H), 1.00 (t, 3H).

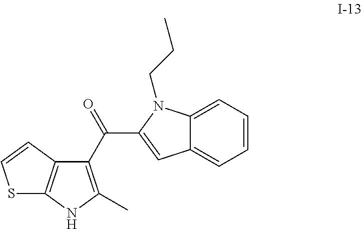 [SMILES: CCCn1c(C(=O)c2c(C)[nH]c3sccc23)cc2ccccc21]

¹H NMR (CDCl₃/400 MHz) δ 8.23 (br s, 1H), 7.78 (s, 1H), 7.72 (d, 1H), 7.45 (d, 1H)., 7.36 (t, 1H), 7.19-7.16 (m, 2H), 6.20 (s, 1H), 4.48 (t, 2H), 2.43 (s, 3H), 1.87 (q, 2H), 0.92 (t, 3H).

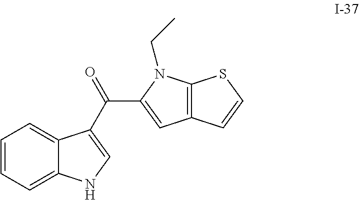 [SMILES: CCn1c(C(=O)c2c[nH]c3ccccc23)cc2ccsc21]

¹H NMR (CDCl₃/400 MHz) δ 8.53 (br s, 1H), 8.41 (t, 1H), 7.87 (d, 1H), 7.45 (d, 1H), 7.33-7.31 (m, 2H), 7.03 (d, 1H), 7.00 (s 1H), 6.94 (d, 1H), 4.55 (q, 2H), 1.54 (t, 3H).

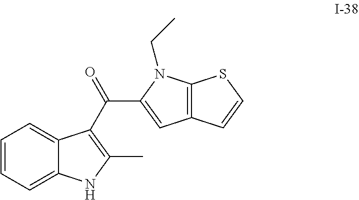 [SMILES: CCn1c(C(=O)c2c(C)[nH]c3ccccc23)cc2ccsc21]

¹H NMR (CDCl₃/400 MHz) δ 8.26 (br s, 1H), 7.77 (d, 1H), 7.34 (d, 1H), 7.18 (d, 1H), 7.11 (t, 1H), 6.97 (d, 1H), 6.93-6.91 (m, 2H), 4.58 (q, 2H), 2.64 (s, 3H), 1.54 (t, 3H).

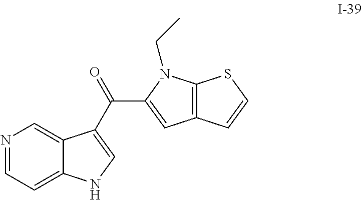 [SMILES: CCn1c(C(=O)c2c[nH]c3ccncc23)cc2ccsc21]

¹H NMR (CDCl₃/400 MHz) δ 9.62 (s, 1H), 8.32 (d, 1H), 8.14 (s, 1H), 7.79 (d, 1H), 7.07 (s, 1H), 6.99 (d, 1H), 6.93 (d, 1H), 4.49 (q, 2H), 1.49 (t, 3H).

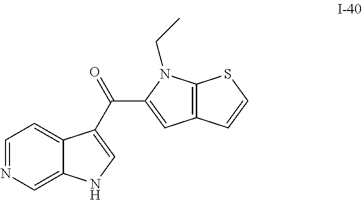 [SMILES: CCn1c(C(=O)c2c[nH]c3cnccc23)cc2ccsc21]

¹H NMR (CDCl₃/400 MHz) δ 8.91 (s, 1H), 8.43 (d, 1H), 8.31 (d, 1H), 8.14 (s, 1H), 7.05 (s, 1H), 7.03 (d, 1H), 6.95 (d, 1H), 4.55 (q, 2H), 1.54 (t, 3H).

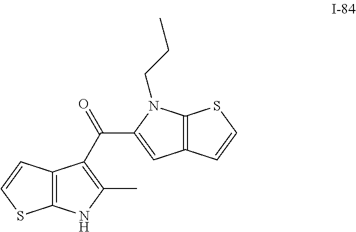 [SMILES: CCCn1c(C(=O)c2c(C)[nH]c3sccc23)cc2ccsc21]

¹H NMR (CDCl₃/400 MHz) δ 8.46 (br s, 1H), 7.04 (s, 1H), 6.99 (d, 2H), 6.91 (d, 1H), 6.81 (d, 1H), 4.81 (t, 2H), 2.66 (s, 3H), 1.95 (q, 2H), 0.95 (t, 3H).

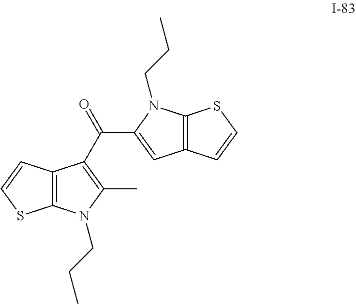 [SMILES: CCCn1c(C(=O)c2c(C)n(CCC)c3sccc23)cc2ccsc21]

¹H NMR (CDCl₃/400 MHz) δ 7.02 (s, 1H), 6.97 (dd, 2H), 6.90 (d, 1H), 6.80 (d, 1H), 4.48 (t, 2H), 3.98 (t, 2H), 2.64 (s, 3H), 1.95-1.90 (m, 4H), 0.99 (t, 3H), 0.94 (t, 3H).

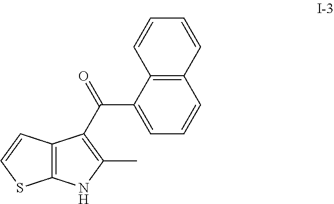 [SMILES: Cc1[nH]c2sccc2c1C(=O)c1cccc2ccccc12]

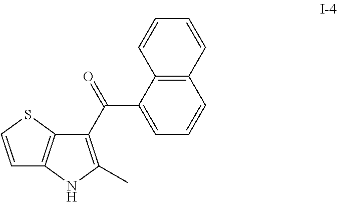 [SMILES: Cc1[nH]c2ccsc2c1C(=O)c1cccc2ccccc12]

¹H NMR (CDCl₃/400 MHz) δ 8.05 (dd, 1H), 8.00 (dd, 1H), 7.96-7.93 (m, 1H), 7.63-7.59 (m, 2H), 7.56-7.52 (m, 1H), 7.49-7.45 (m, 1H), 7.07 (d, 1H), 6.94 (d, 1H), 2.34 (s, 3H).

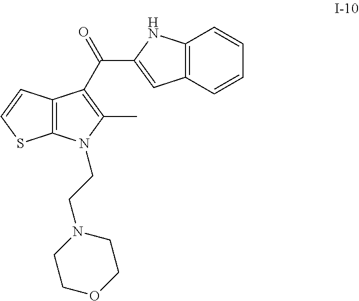 [SMILES: Cc1c(C(=O)c2cc3ccccc3[nH]2)c2ccsc2n1CCN1CCOCC1]

¹H NMR (CDCl₃/400 MHz) δ 9.18 (br s, 1H), 7.69 (d, 1H), 7.47 (dd, 1H), 7.34 (td, 2H), 7.12-7.13 (m, 2H), 6.89 (d, 1H), 4.14 (t, 2H), 3.72 (t, 4H), 2.79 (t, 2H), 2.69 (s, 3H), 2.54 (t, 4H); MS m/z: 394.2 (M+1).

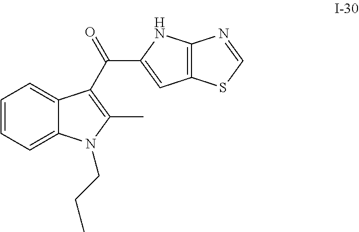 [SMILES: CCCn1c(C)c(C(=O)c2cc3scnc3[nH]2)c2ccccc21]

¹H NMR (CDCl₃/400 MHz) δ 8.76 (s, 1H), 7.84 (d, 1H), 7.35 (d, 1H), 7.23 (t, 1H), 7.14 (td, 1H), 7.09 (s, 1H), 4.13 (t, 2H), 2.68 (s, 3H), 1.86 (q, 2H), 1.02 (t, 3H).

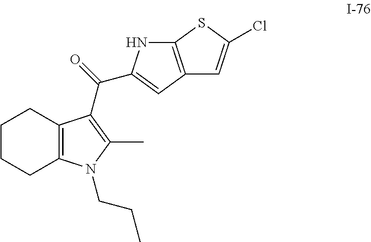 [SMILES: CCCn1c(C)c(C(=O)c2cc3cc(Cl)sc3[nH]2)c2c1CCCC2]

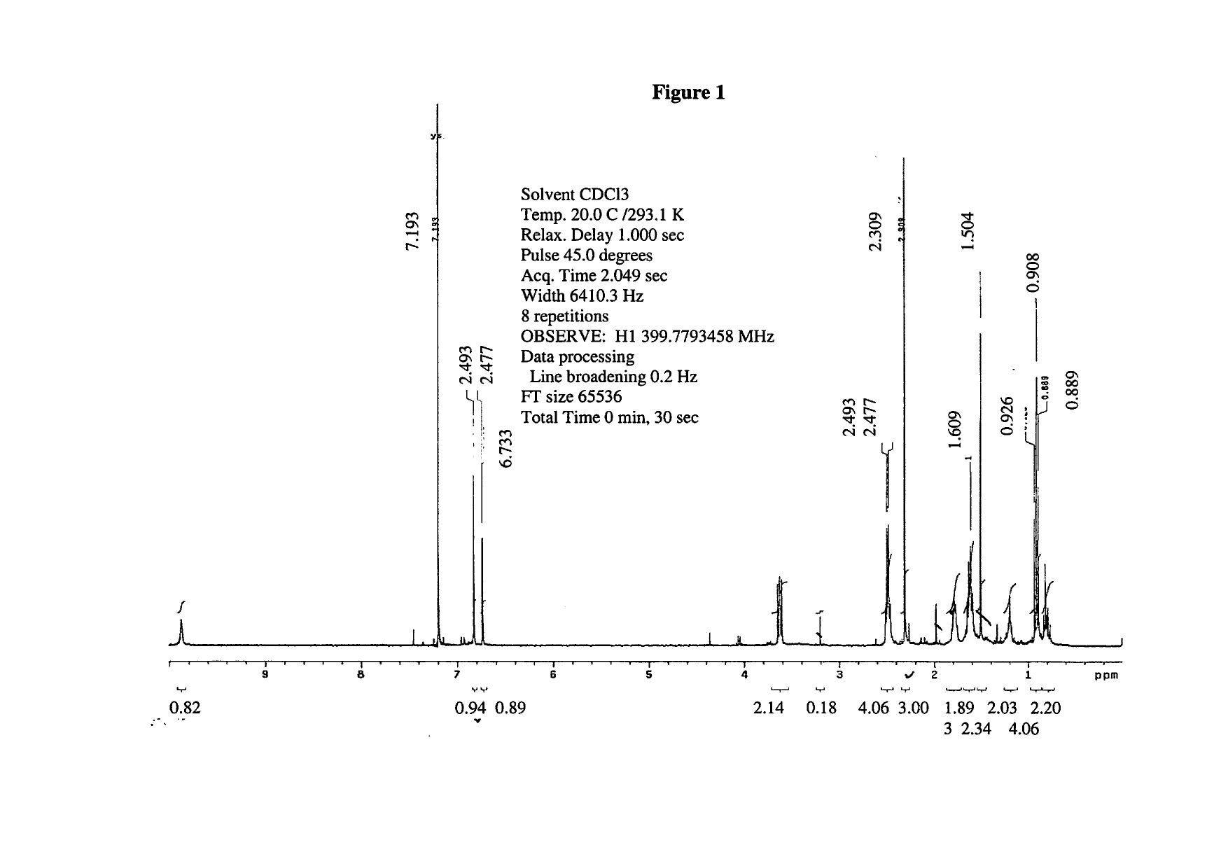

See FIG. 1 for NMR.

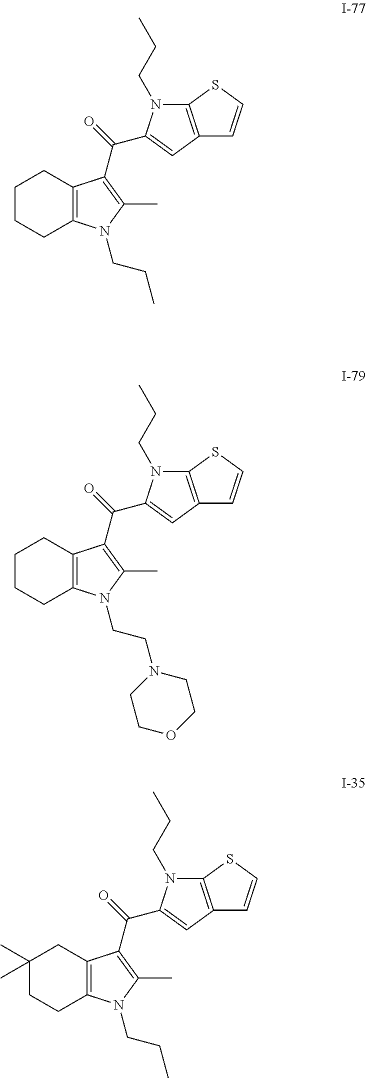 [SMILES: CCCn1c(C)c(C(=O)c2cc3ccsc3n2CCC)c2c1CCCC2 ; CCCn1c(C(=O)c2c3c(n(CCN4CCOCC4)c2C)CCCC3)cc2ccsc21 ; CCCn1c(C)c(C(=O)c2cc3ccsc3n2CCC)c2c1CCC(C)(C)C2]

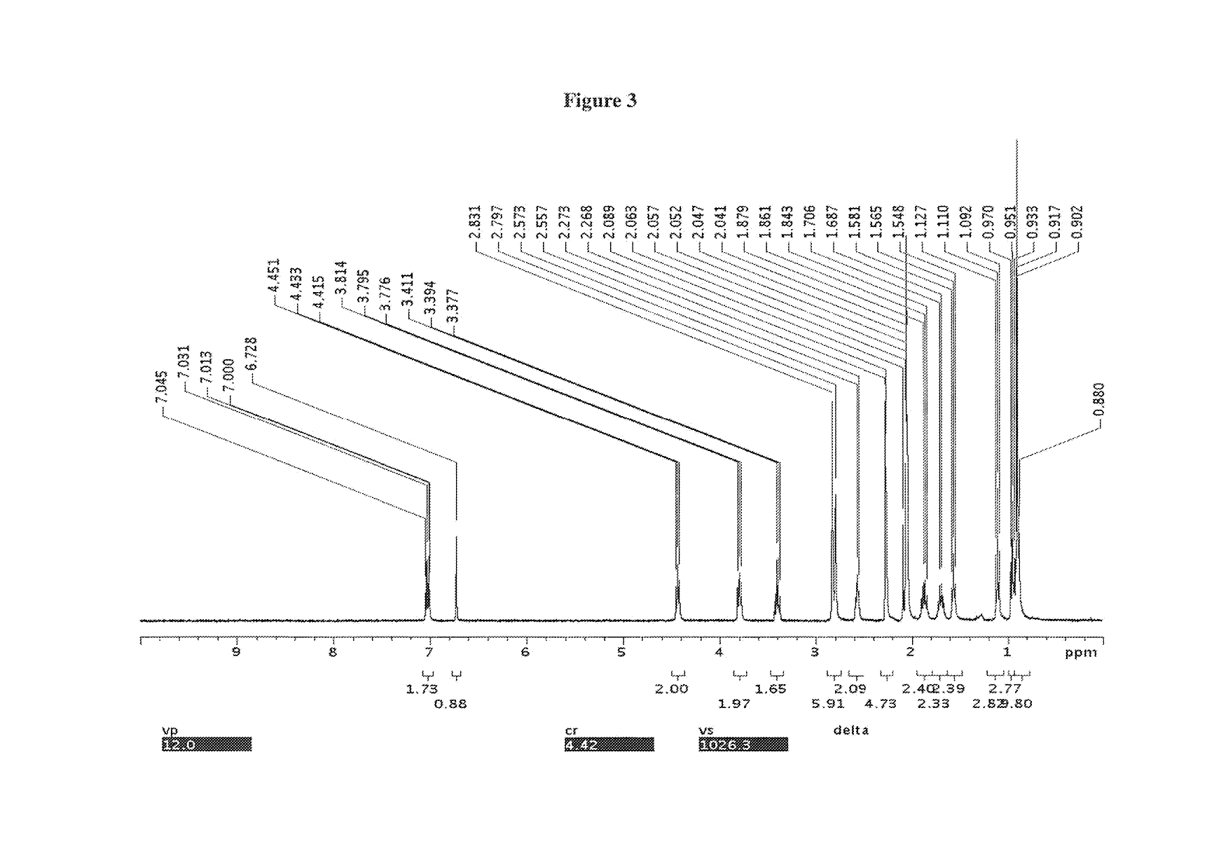

See FIG. 3 for NMR.

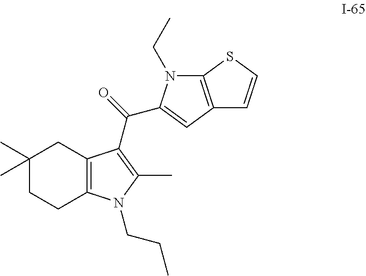 [SMILES: CCCn1c(C)c(C(=O)c2cc3ccsc3n2CC)c2c1CCC(C)(C)C2]

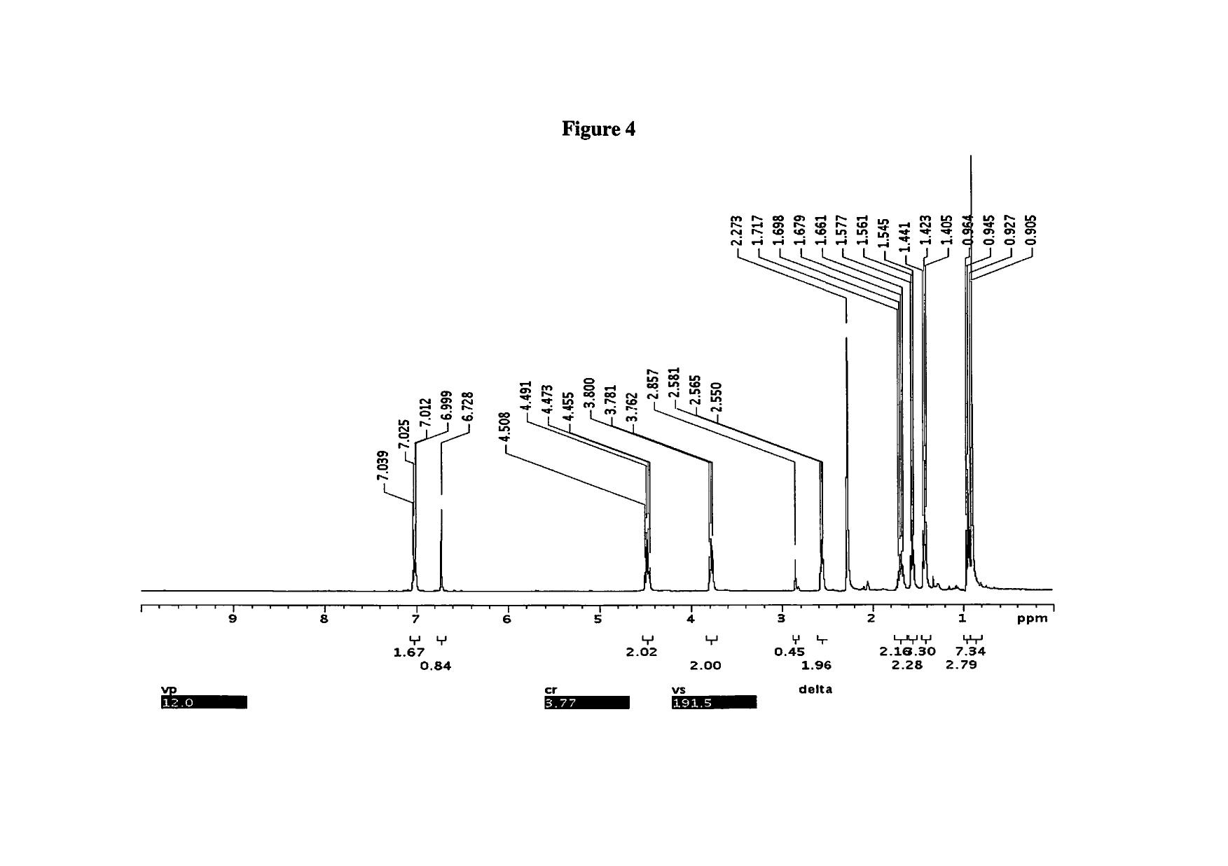

See FIG. 4 for NMR

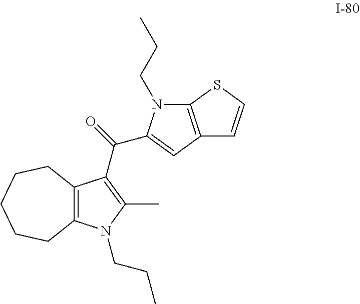 [SMILES: CCCn1c(C)c(C(=O)c2cc3ccsc3n2CCC)c2c1CCCCC2]

¹H NMR (CDCl₃/400 MHz) δ 6.96 (d, 1H), 6.87 (d, 1H), 6.81 (s, 1H), 4.45 (t, 2H), 3.68 (t, 2H), 2.53-2.48 (m, 4H), 2.31 (s, 3H), 1.89 (q, 2H), 1.82-1.81 (m, 2H), 1.70-1.65 (m, 4H), 1.56 (br s, 2H), 0.98-0.86 (s, 6H).

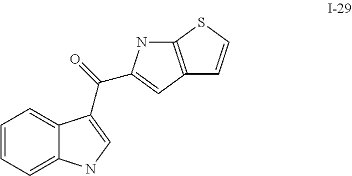 [SMILES: O=C(c1cc2ccsc2[nH]1)c1c[nH]c2ccccc12]

### Example 8

### N-Alkylation after Coupling

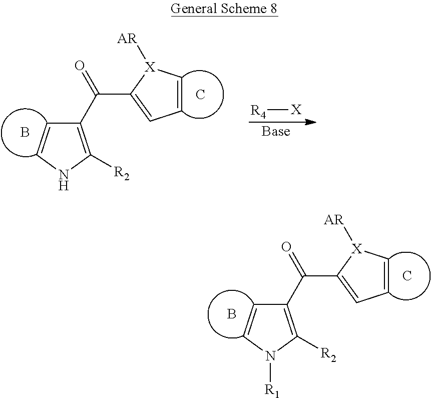

Preparation of Compound x

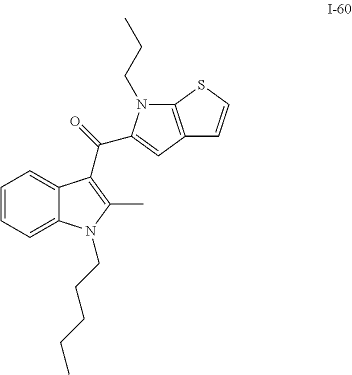 [SMILES: CCCCCn1c(C)c(C(=O)c2cc3ccsc3n2CCC)c2ccccc21]

To a solution of (2-methyl-1H-indol-3-yl)(6-propyl-6H-thieno[2,3-b]pyrrol-5-yl)methanone (150 mg, 0.46 mmol) in DMF (300 mL) at room temperature was added KOH (456 mg, 2.3 mmol), followed by the addition of 1-iodopentane (456 mg, 2.3 mmol). The reaction mixture was stirred overnight at room temperature. The reaction was then quenched by addition of water (200 mL). The resulting mixture was extracted with DCM (3×50 mL). The organic layers were combined, washed with brine, dried over MgSO₄, filtered and concentrated under reduced pressure. The crude product was further purified by flash chromatography (20-70% EtOAc in Hexanes) to give (2-methyl-1-pentyl-1H-indol-3-yl)(6-propyl-6H-thieno[2,3-b]pyrrol-5-yl)methanone (54 mg, 30%). ¹H NMR (CDCl₃/400 MHz) δ 7.58 (d, 1H), 7.42 (d, 1H), 7.18 (td, 1H), 7.04 (td, 1H), 7.01 (d, 1H), 6.95 (d, 1H), 6.83 (s, 1H), 4.64 (t, 2H), 4.22 (t, 2H), 2.57 (s, 3H), 1.92 (q, 2H), 1.82-1.79 (m, 2H), 1.40-1.37 (m, 4H), 0.94-0.92 (m, 6H).

The following compounds were prepared according to Example 8 and General Scheme 8:

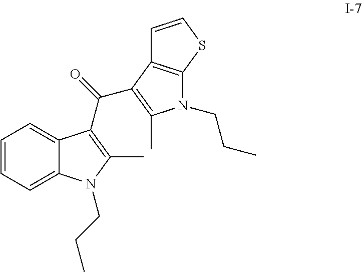 [SMILES: CCCn1c(C)c(C(=O)c2c(C)n(CCC)c3sccc23)c2ccccc21]

¹H NMR (CDCl₃/400 MHz) δ 7.51 (d, 1H), 7.31 (d, 1H), 7.16 (td, 1H), 7.02 (td, 1H), 6.68 (d, 1H), 6.42 (d, 1H), 4.10 (t, 2H), 3.98 (t, 2H), 2.58 (s, 3H), 2.54 (s, 3H), 1.91 (q, 2H), 1.84 (q, 2H), 0.99 (t, 6H).

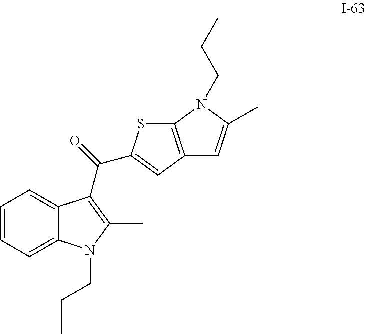 [SMILES: CCCn1c(C)c(C(=O)c2cc3cc(C)n(CCC)c3s2)c2ccccc21]

¹H NMR (CDCl₃/400 MHz) δ 7.68 (d, 1H), 7.46 (s, 1H), 7.26 (d, 1H), 7.13 (t, 1H), 7.03 (t, 1H), 6.05 (d, 1H), 4.04 (t, 2H), 3.86 (t, 2H), 2.56 (s, 3H), 2.29 (d, 3H), 1.86-1.75 (m, 4H), 0.96-0.89 (m, 6H).

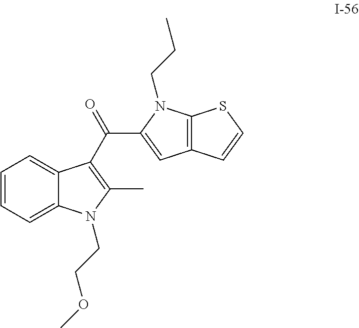 [SMILES: CCCn1c(C(=O)c2c(C)n(CCOC)c3ccccc23)cc2ccsc21]

¹H NMR (MeOD/400 MHz) δ 7.58 (d, 1H), 7.43 (d, 1H), 7.17 (td, 1H), 7.05 (td, 1H), 7.01 (d, 1H), 6.95 (d, 1H), 6.84 (s, 1H), 4.46 (t, 2H), 4.41 (t, 2H), 3.73 (t, 2H), 3.27 (s, 3H), 2.58 (s, 3H), 1.92 (q, 2H), 0.93 (t, 3H).

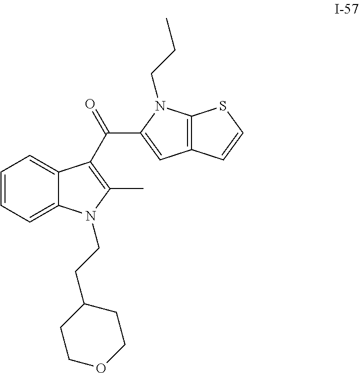 [SMILES: CCCn1c(C(=O)c2c(C)n(CCC3CCOCC3)c3ccccc23)cc2ccsc21]

¹H NMR (MeOD/400 MHz) δ 7.59 (d, 1H), 7.42 (d, 1H), 7.19 (td, 1H), 7.06 (t, 1H), 7.02 (d, 1H), 6.96 (d, 1H), 6.84 (s, 1H), 4.47 (t, 2H), 4.27 (t, 2H), 3.94 (dd, 2H), 3.43 (t, 2H), 2.58 (s, 3H), 1.93 (q, 2H), 1.75-1.72 (m, 5H), 1.44-1.32 (m, 2H), 0.94 (t, 3H).

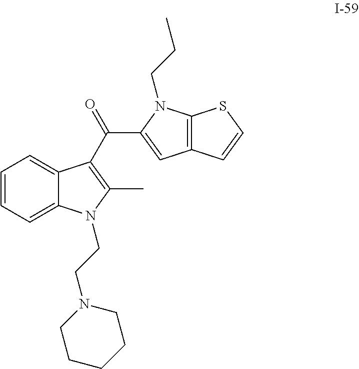 [SMILES: CCCn1c(C(=O)c2c(C)n(CCN3CCCCC3)c3ccccc23)cc2ccsc21]

¹H NMR (MeOD/400 MHz) δ 7.60 (d, 1H), 7.46 (d, 1H), 7.20 (td, 1H), 7.07 (td, 1H), 7.02 (d, 1H), 6.96 (d, 1H), 6.84 (s, 1H), 4.48 (t, 2H) 4.41 (t, 2H), 2.76 (t, 2H), 2.64 (br s, 4H), 2.60 (s, 3H), 1.92 (q, 2H), 1.69-1.64 (m, 4H), 1.53-1.51 (m, 2H), 0.92 (t, 3H).

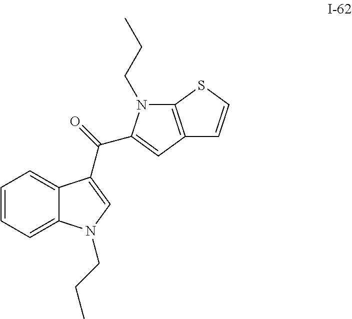 [SMILES: CCCn1cc(C(=O)c2cc3ccsc3n2CCC)c2ccccc21]

¹H NMR (MeOD/400 MHz) 8.26 (d, 1H), 8.00 (s, 1H), 7.53 (d, 1H), 7.32 (td, 1H), 7.26 (td, 1H), 7.05-7.04 (m, 2H), 4.44 (t, 2H), 4.26 (t, 2H), 1.96-1.87 (m, 4H), 0.97-0.91 (m, 6H).

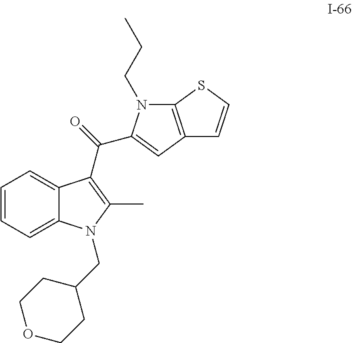 [SMILES: CCCn1c(C(=O)c2c(C)n(CC3CCOCC3)c3ccccc23)cc2ccsc21]

¹H NMR (MeOD/400 MHz) δ 7.61 (d, 1H), 7.48 (d, 1H), 7.21 (t, 1H), 7.08 (t, 1H), 7.03 (d, 1H), 6.97 (d, 1H), 6.85 (s, 1H), 4.50 (t, 2H), 4.15 (d, 2H), 3.94 (d, 1H), 3.39-3.34 (m, 2H), 2.60 (s, 3H), 2.28-2.18 (m, 1H), 1.95 (q, 2H), 1.54-1.51 (m, 4H), 0.96 (t, 3H).

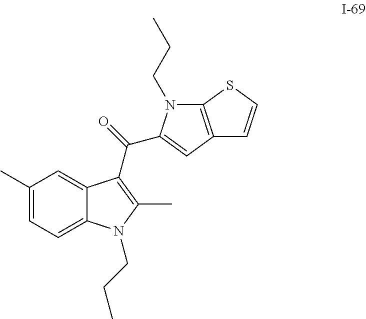 [SMILES: CCCn1c(C)c(C(=O)c2cc3ccsc3n2CCC)c2cc(C)ccc21]

¹H NMR (CDCl₃/400 MHz) δ 7.57 (s, 1H), 7.21 (d, 1H), 7.02 (d, 1H), 6.95 (d, 1H), 6.90 (d, 1H), 6.87 (s, 1H), 4.52 (t, 2H), 4.08 (t, 2H), 2.57 (s, 3H), 2.38 (s, 3H), 1.98 (q, 2H), 1.84 (t, 2H), 1.02-0.96 (m, 6H).

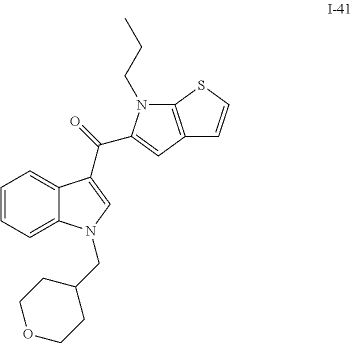 [SMILES: CCCn1c(C(=O)c2cn(CC3CCOCC3)c3ccccc23)cc2ccsc21]

¹H NMR (CDCl₃/400 MHz) δ 8.40 (dd, 1H), 7.72 (s, 1H), 7.39 (dd, 1H), 7.36-7.29 (m, 2H), 7.03 (d, 1H), 6.95-6.93 (m, 2H), 4.46 (t, 2H), 4.07 (d, 2H), 3.98 (dd, 2H), 3.33 (td, 2H), 2.20-2.12 (m, 1H), 1.97 (q, 2H), 1.54 (d, 2H), 1.49-1.38 (m, 3H), 0.97 (t, 3H).

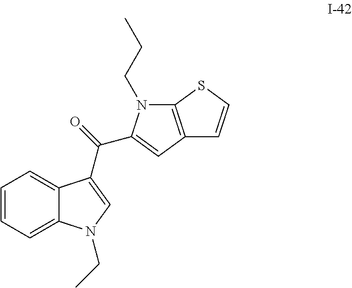 [SMILES: CCCn1c(C(=O)c2cn(CC)c3ccccc23)cc2ccsc21]

¹H NMR (CDCl₃/400 MHz) δ 8.40 (dd, 1H), 7.80 (s, 1H), 7.41 (d, 1H), 7.34-7.31 (m, 2H), 7.03 (d, 1H), 6.97 (s, 1H), 6.93 (d, 1H), 4.46 (t, 2H), 4.25 (q, 2H), 1.96 (q, 2H), 1.55 (t, 3H), 0.96 (t, 3H).

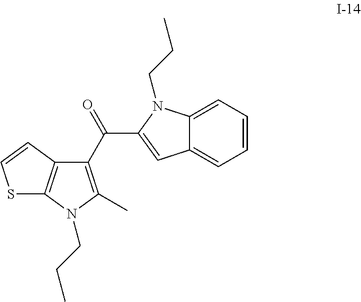 [SMILES: CCCn1c(C(=O)c2c(C)n(CCC)c3sccc23)cc2ccccc21]

¹H NMR (CDCl₃/400 MHz) δ 7.65 (d, 1H), 7.45 (d, 1H), 7.34 (td, 1H), 7.14 (t, 1H), 7.08 (s, 1H), 6.84 (d, 1H), 6.79 (d, 1H), 4.55 (t, 2H), 4.03 (t, 2H), 2.68 (d, 3H), 1.95-1.84 (m, 4H), 1.02 (t, 3H), 0.91 (t, 3H).

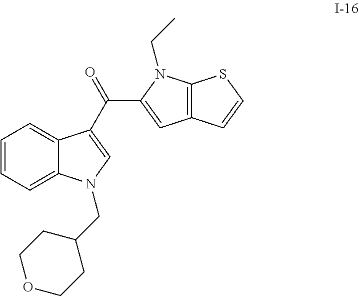 [SMILES: CCn1c(C(=O)c2cn(CC3CCOCC3)c3ccccc23)cc2ccsc21]

¹H NMR (CDCl₃/400 MHz) δ 8.42 (d, 1H), 7.74 (s, 1H), 7.40-7.32 (m, 3H), 7.04 (d, 1H), 6.96-6.94 (m, 2H), 4.54 (q, 2H), 4.08 (d, 2H), 3.99-3.97 (m, 2H), 3.33 (t, 2H), 2.16 (m, 1H), 1.56-1.52 (m, 5H), 1.49-1.40 (m, 2H).

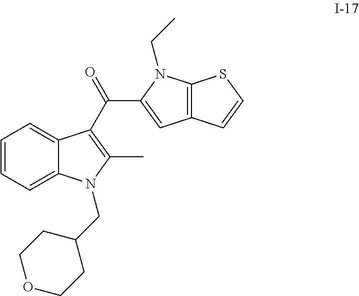 [SMILES: CCn1c(C(=O)c2c(C)n(CC3CCOCC3)c3ccccc23)cc2ccsc21]

¹H NMR (CDCl₃/400 MHz) δ 7.73 (d, 1H), 7.31 (d, 1H), 7.21 (d, 1H), 7.10 (t, 1H), 6.95 (d, 1H), 6.92 (d, 1H), 6.88 (s, 1H), 4.59 (q, 2H), 4.05 (d, 2H), 3.99 (d, 2H), 3.33 (t, 2H), 2.64 (s, 3H), 2.18 (m, 1H), 1.54-1.49 (m, 7H).

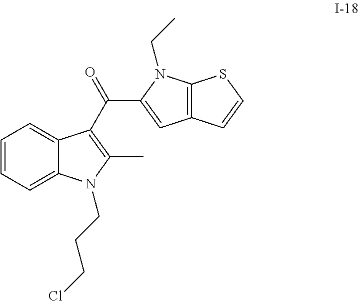 [SMILES: CCn1c(C(=O)c2c(C)n(CCCCl)c3ccccc23)cc2ccsc21]

¹H NMR (CDCl₃/400 MHz) δ 7.74 (d, 1H), 7.38 (d, 1H), 7.21 (t, 1H), 7.10 (t, 1H), 6.95 (dd, 1H), 6.91 (dd, 1H), 6.88 (d, 1H), 4.58 (q, 2H), 4.34 (t, 2H), 3.58 (t, 2H), 2.65 (d, 3H), 2.78 (t, 2H), 1.54 (t, 3H).

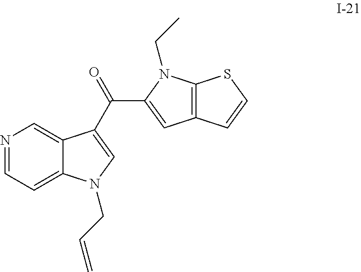 [SMILES: C=CCn1cc(C(=O)c2cc3ccsc3n2CC)c2cnccc21]

¹H NMR (CDCl₃/400 MHz) δ 9.61 (s, 1H), 8.47 (d, 1H), 7.78 (s, 1H), 7.30 (d, 1H), 7.04-7.02 (m, 2H), 6.96 (d, 1H), 6.08-5.99 (m, 1H), 5.35 (dd, 1H), 5.20 (dd, 1H), 4.81 (dd, 2H), 4.55 (q, 2H), 1.54 (t, 3H).

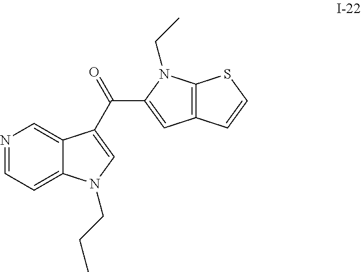 [SMILES: CCCn1cc(C(=O)c2cc3ccsc3n2CC)c2cnccc21]

¹H NMR (CDCl₃/400 MHz) δ 9.60 (s, 1H), 8.47 (d, 1H), 7.78 (s, 1H), 7.32 (d, 1H), 7.04-7.01 (m, 2H), 6.96 (d, 1H), 4.55 (q, 2H), 4.16 (t, 2H), 1.94 (q, 2H), 1.54 (t, 3H), 0.99 (t, 3H).

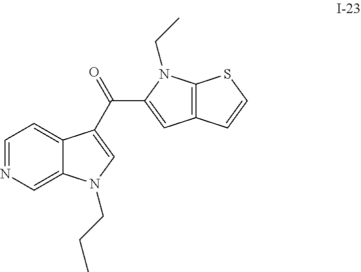 [SMILES: CCCn1cc(C(=O)c2cc3ccsc3n2CC)c2ccncc21]

¹H NMR (CDCl₃/400 MHz) δ 8.86 (s, 1H), 8.44 (d, 1H), 8.22 (d, 1H), 7.89 (s, 1H), 7.04 (d, 1H), 6.98-6.95 (m, 2H), 4.54 (q, 2H), 4.26 (t, 2H), 2.00 (q, 2H), 1.54 (t, 3H), 0.99 (t, 3H).

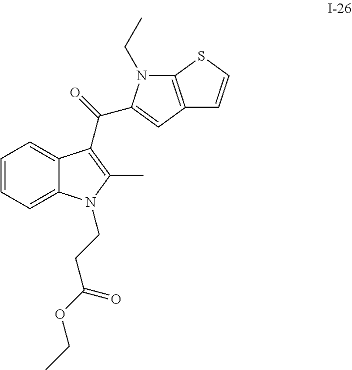 [SMILES: CCOC(=O)CCn1c(C)c(C(=O)c2cc3ccsc3n2CC)c2ccccc21]

¹H NMR (CDCl₃/400 MHz) δ 7.74 (d, 1H), 7.36 (d, 1H), 7.22 (t, 1H), 7.11 (t, 1H), 6.95 (d, 1H), 6.92 (d, 1H), 6.88 (s, 1H), 4.59 (q, 2H), 4.50 (t, 2H), 4.17 (t, 2H), 2.81 (t, 2H), 2.65 (s, 3H), 1.54 (t, 3H), 1.25 (t, 3H).

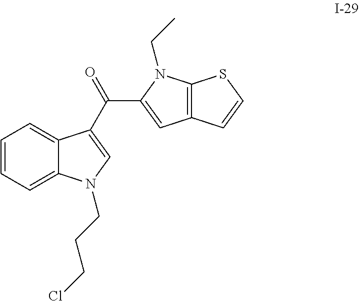 [SMILES: CCn1c(C(=O)c2cn(CCCCl)c3ccccc23)cc2ccsc21]

¹H NMR (CDCl₃/400 MHz) δ 8.42 (dd, 1H), 7.82 (s, 1H), 7.44 (dd, 1H), 7.37-7.32 (m, 2H), 7.04 (d, 1H), 6.98 (s, 1H), 6.95 (d, 1H), 4.54 (q, 2H), 4.44 (t, 2H), 3.51 (t, 2H), 2.35 (t, 2H), 1.54 (t, 3H).

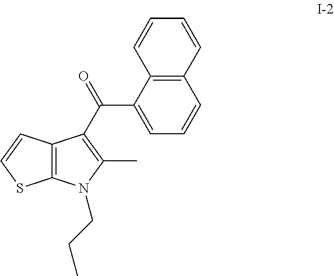 [SMILES: CCCn1c(C)c(C(=O)c2cccc3ccccc23)c2ccsc21]

¹H NMR (CDCl₃/400 MHz) δ 8.07 (dd, 1H), 7.94 (d, 1H), 7.89 (dd, 1H), 7.58 (dd, 1H), 7.51-7.40 (m, 3H), 6.60 (d, 1H), 5.83 (d, 2H), 3.97 (t, 2H), 2.69 (s, 3H), 1.89 (q, 2H), 0.98 (t, 3H).

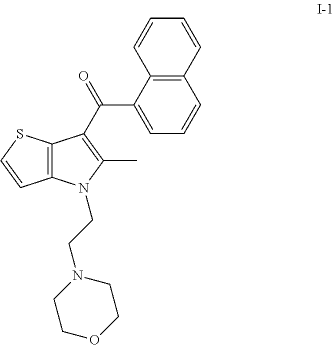 [SMILES: Cc1c(C(=O)c2cccc3ccccc23)c2sccc2n1CCN1CCOCC1]

¹H NMR (CDCl₃/400 MHz) δ 8.02 (d, 1H), 7.96 (d, 1H), 7.89 (d, 1H), 7.60 (dd, 1H), 7.53 (d, 1H), 7.51-7.46 (m, 1H), 7.48-7.40 (m, 1H), 6.92 (d, 1H), 6.86 (d, 1H), 4.16 (t, 2H), 3.68 (t, 4H), 2.69 (t, 2H), 2.59 (s, 3H), 2.48 (t, 4H).

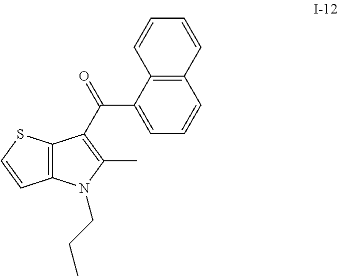 [SMILES: CCCn1c(C)c(C(=O)c2cccc3ccccc23)c2sccc21]

¹H NMR (CDCl₃/400 MHz) δ 8.03 (dd, 1H), 7.96 (d, 1H), 7.89 (d, 1H), 7.60 (dd, 1H), 7.54 (dd, 1H), 7.50-7.46 (m, 1H), 7.44-7.40 (m, 1H), 6.90 (d, 1H), 6.84 (d, 1H), 4.00 (t, 2H), 2.58 (s, 3H), 1.82 (q, 2H), 0.96 (t, 3H).

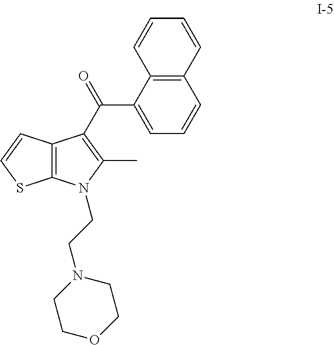 [SMILES: Cc1c(C(=O)c2cccc3ccccc23)c2ccsc2n1CCN1CCOCC1]

¹H NMR (CDCl₃/400 MHz) δ 8.06 (d, 1H), 7.94 (d, 1H), 7.88 (d, 1H), 7.57 (dd, 1H), 7.51-7.40 (m, 3H), 6.61 (d, 1H), 5.85 (t, 1H), 4.10 (t, 2H), 3.69 (t, 4H), 2.75 (t, 2H), 2.69 (s, 3H), 2.50 (t, 4H).

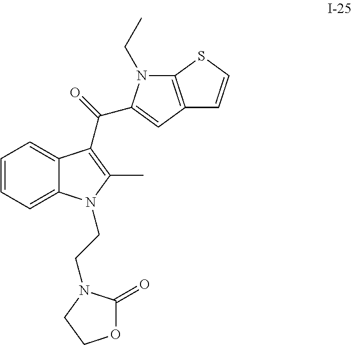 [SMILES: CCn1c(C(=O)c2c(C)n(CCN3CCOC3=O)c3ccccc23)cc2ccsc21]

¹H NMR (CDCl₃/400 MHz) δ 7.72 (d, 1H), 7.39 (d, 1H), 7.22 (t, 1H), 7.11 (t, 1H), 6.95-6.91 (m, 2H), 6.84 (s, 1H), 4.58 (q, 2H), 4.42 (t, 2H), 4.06 (t, 2H), 3.64 (t, 2H), 3.02 (t, 2H), 2.64 (s, 3H), 1.54 (t, 3H).

### Example 9

### Second N-Alkylation of Coupled System

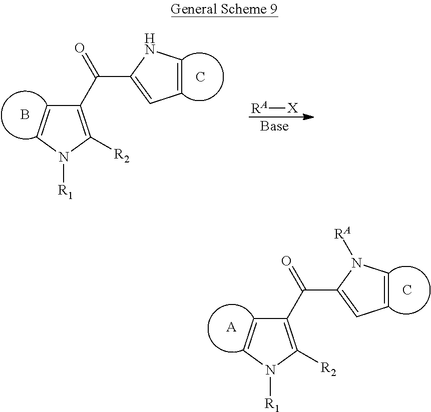

Preparation of Compound xi:

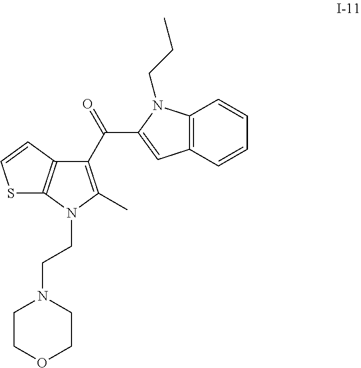 [SMILES: CCCn1c(C(=O)c2c(C)n(CCN3CCOCC3)c3sccc23)cc2ccccc21]

A 40 mL vial equipped with a stir bar was charged with (1H-indol-2-yl)(5-methyl-6-(2-morpholinoethyl)-6H-thieno[2,3-b]pyrrol-4-yl)methanone (86.8 mg, 0.221 mmol) and DMF (1.103 ml). To this mixture was added sodium hydride (17.64 mg, 0.441 mmol). The mixture was then cooled to 0° C. 1-iodopropane (0.026 ml, 0.265 mmol) was then added. The reaction mixture was allowed to warm to room temperature and stirred for 18 h. The reaction mixture was taken up in EtOAc (100 mL) and washed with water (50 ml×5). The organic layer was dried over Na₂SO₄, filtered, and concentrated under reduced pressure to give an oil. The oil was purified with automatic chromatography on silica using a Biotage system (10 g, 2% Et₃N, 0% to 100% EtOAc in hexanes). The purified material was further recrystallized from a mixture of 10:1 hexanes:Et₂O at 0° C. to give (5-methyl-6-(2-morpholinoethyl)-6H-thieno[2,3-b]pyrrol-4-yl)(1-propyl-1H-indol-2-yl)methanone (13.7 mg, 0.031 mmol, 14.26% yield) as a yellow solid. ¹H NMR (CDCl₃/400 MHz) δ 7.65 (d, 1H), 7.45 (d, 1H), 7.35 (d, 1H), 7.14 (t, 1H), 7.07 (s, 1H), 6.84 (d, 1H), 6.79 (d, 1H), 4.55 (t, 2H), 4.14 (t, 2H), 3.73 (t, 4H), 2.79 (t, 2H), 2.70 (s, 3H), 2.54 (t, 4H), 1.86 (q, 2H), 0.91 (t, 3H).

The following compounds were prepared following Example 9 and General Scheme 9:

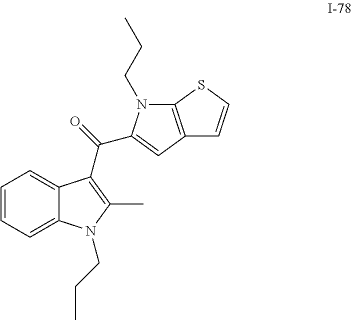 [SMILES: CCCn1c(C)c(C(=O)c2cc3ccsc3n2CCC)c2ccccc21]

¹H NMR (CDCl₃/400 MHz) δ 7.70 (d, 1H), 7.31 (d, 1H), 7.18 (td, 1H), 7.07 (td, 1H), 6.93 (d, 1H), 6.89 (d, 1H), 6.87 (s, 1H), 4.50 (t, 2H), 4.10 (t, 2H), 2.61 (s, 3H), 1.95 (q, 2H), 1.84 (t, 3H), 1.02-0.94 (m, 3H).

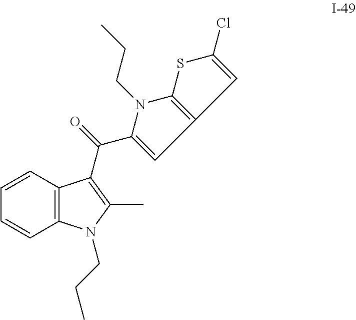 [SMILES: CCCn1c(C)c(C(=O)c2cc3cc(Cl)sc3n2CCC)c2ccccc21]

¹H NMR (CDCl₃/400 MHz) δ 7.47 (d, 1H), 7.35 (d, 1H), 7.11-7.07 (m, 1H), 6.96 (td, 1H), 6.87 (s, 1H), 6.92 (s, 1H), 4.34 (t, 2H), 4.12 (t, 2H), 2.49 (s, 3H), 1.84-1.73 (m, 4H), 0.91 (t, 3H), 0.84 (t, 3H).

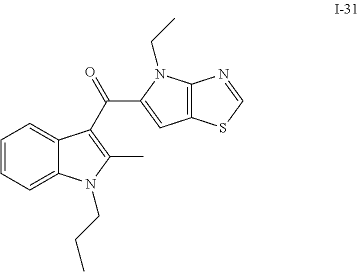 [SMILES: CCCn1c(C)c(C(=O)c2cc3scnc3n2CC)c2ccccc21]

¹H NMR (CDCl₃/400 MHz) δ 8.73 (s, 1H), 7.67 (d, 1H), 7.33 (d, 1H), 7.21 (t, 1H), 7.10 (t, 1H), 6.89 (s, 1H), 4.80 (q, 2H), 4.13 (t, 2H), 2.64 (s, 3H), 1.86 (q, 2H), 1.55 (t, 3H), 1.02 (t, 3H).

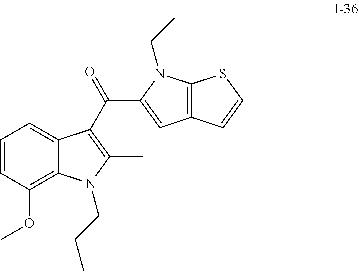 [SMILES: CCCn1c(C)c(C(=O)c2cc3ccsc3n2CC)c2cccc(OC)c21]

¹H NMR (Acetone-d6/400 MHz) δ 7.99 (d, 1H), 7.97 (d, 1H), 7.15 (t, 1H), 7.08 (dd, 1H), 7.04 (s, 1H), 6.84 (d, 1H), 4.50 (td, 4H), 4.00 (s, 3H), 1.91 (q, 2H), 1.46 (t, 3H), 0.93 (t, 3H).

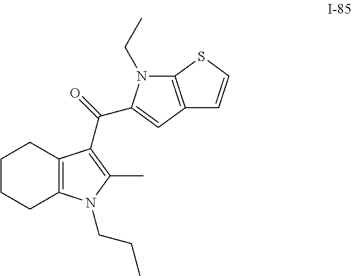 [SMILES: CCCn1c(C)c(C(=O)c2cc3ccsc3n2CC)c2c1CCCC2]

¹H NMR (CDCl₃/400 MHz) δ 6.97 (d, 1H), 6.88 (d, 1H), 6.81 (s, 1H), 4.50 (q, 2H), 3.68 (t, 2H), 2.55-2.47 (m, 4H), 2.31 (s, 3H), 1.84-1.81 (m, 2H), 1.71-1.63 (m, 4H), 1.46 (t, 3H), 0.97 (t, 3H).

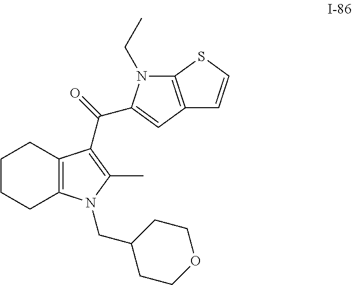 [SMILES: CCn1c(C(=O)c2c3c(n(CC4CCOCC4)c2C)CCCC3)cc2ccsc21]

¹H NMR (CDCl₃/400 MHz) δ 6.97 (d, 1H), 6.89 (d, 1H), 6.79 (s, 1H), 4.50 (q, 2H), 4.00 (dd, 2H), 3.61 (d, 2H), 3.36 (td, 2H), 2.53-2.46 (m, 4H), 2.31 (s, 3H), 1.98-1.88 (m, 1H), 1.84-1.81 (m, 2H), 1.66-1.63 (m, 2H), 1.55 (br s, 1H), 1.48-1.44 (m, 6H).

### Example 10

### N-Sulfonylation

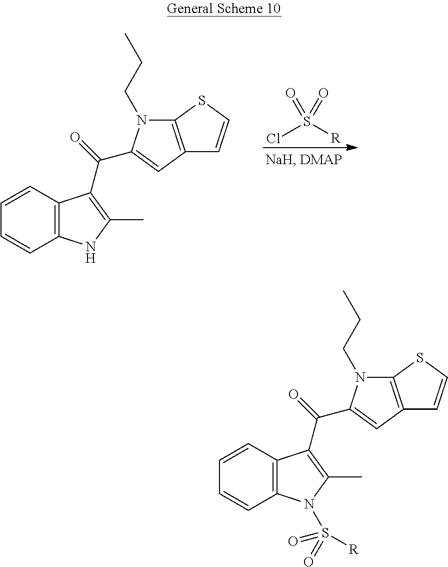

Preparation of Compound I-67

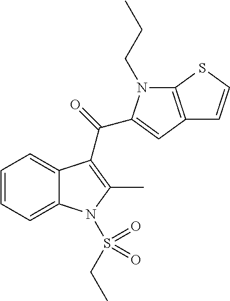 [SMILES: CCCn1c(C(=O)c2c(C)n(S(=O)(=O)CC)c3ccccc23)cc2ccsc21]

¹H NMR (MeOD/400 MHz) δ 8.07 (d, 1H), 7.51 (d, 1H), 7.35 (t, 1H), 7.27 (t, 1H), 7.09 (d, 1H), 6.99 (d, 1H), 6.89 (s, 1H), 4.59 (t, 2H), 3.54 (t, 2H), 2.68 (s, 3H), 2.01 (q, 2H), 1.28 (t, 3H), 1.02 (t, 3H).

Preparation of Compound I-87

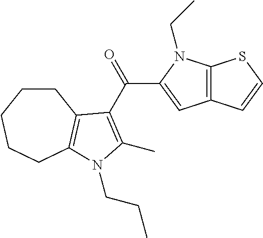 [SMILES: CCCn1c(C)c(C(=O)c2cc3ccsc3n2CC)c2c1CCCCC2]

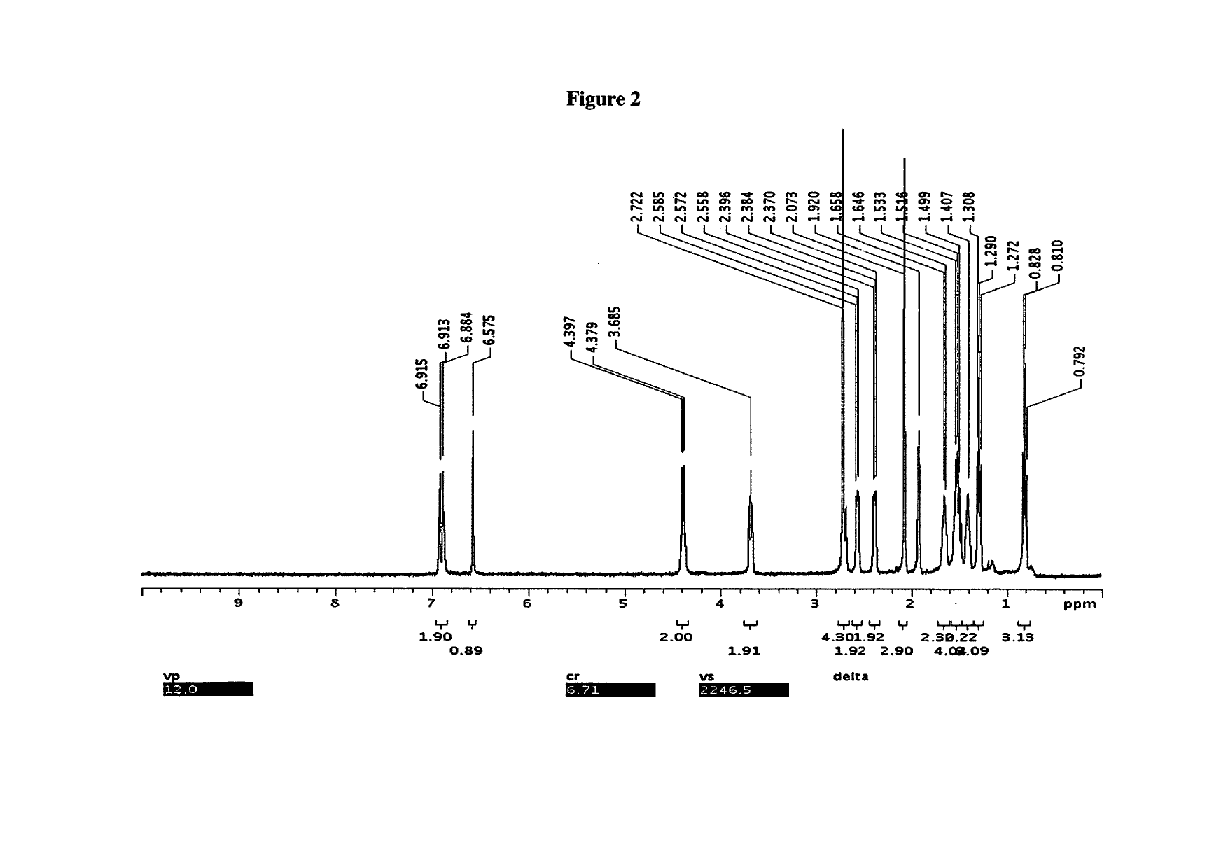

See FIG. 2 for NMR.

### Example 11

### Formation of Thioketone

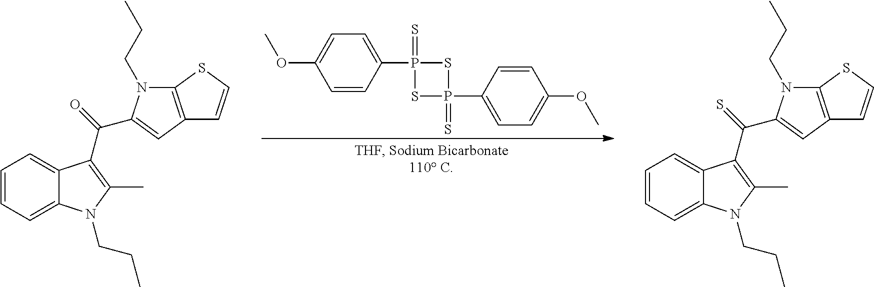

To a microwave vial (20 mL) was added (2-methyl-1-propyl-1H-indol-3-yl)(6-propyl-6H-thieno[2,3-b]pyrrol-5-yl)methanone (20 mg, 0.055 mmol), Lawesson's reagent (13.32 mg, 0.033 mmol) and sodium bicarbonate (9.22 mg, 0.110 mmol) in TI-IF (2 ml), at room temperature. The resulting mixture was heated at 100° C. in a microwave oven for 20 min. The reaction was poured onto water (50 mL) and extracted with DCM (3×10 mL). The organic layers were combined, dried over MgSO₄, filtered and concentrated under vacuum to provide a dark brown oil crude product. The crude product was chromatographed by dry loading the sample and eluting with a gradient of 10% to 40% EtOAc/hexanes onto silica gel. The product was obtained as a bright yellow solid (18.2 mg, 87%). ¹H NMR (MeOD/400 MHz) δ 7.52 (d, 1H), 7.38 (d, 1H), 7.13 (t, 1H), 6.99 (t, 1H), 6.93 (dd, 1H), 6.68 (s, 1H), 4.17-4.09 (m, 4H), 2.36 (s, 3H), 1.82-1.77 (m, 2H), 1.71-1.64 (m, 2H), 0.94 (t, 3H), 0.71 (t, 3H).

### Example 12

### Formation of Sulfonamides

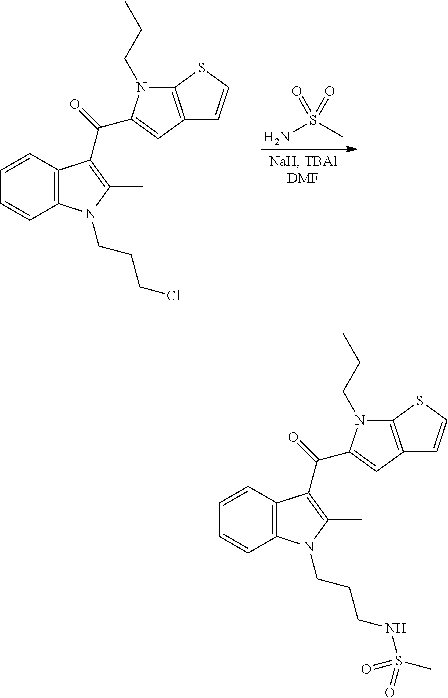

(1-(3-chloropropyl)-2-methyl-1H-indol-3-yl)(6-ethyl-6H-thieno[2,3-b]pyrrol-5-yl)methanone (70 mg, 0.182 mmol) was diluted in DMF (2 mL) at 0° C. and sodium hydride (196 mg, 4.91 mmol) was added. The mixture was stirred at 0° C. for 1 h. To the resulting suspension was added THF (2 mL). The suspension was heated at 50° C. for 20 minutes and then allowed to cool back to room temperature. A solution of (1-(3-chloropropyl)-2-methyl-1H-indol-3-yl)(6-ethyl-6H-thieno[2,3-b]pyrrol-5-yl)methanone (70 mg, 0.182 mmol) in DMF (5 mL) was then added via syringe. The resulting yellow suspension was stirred at room temperature for 40 min. Monitoring by LC/MS showed only a dimer. Tetrabutylammonium iodide (67.2 mg, 0.182 mmol) was added and the suspension was stirred at room temperature for 18 h. The reaction did not show completion after overnight. So it was heated at 60° C. for an additional 3 h, after which, LC/MS showed complete transformation. The reaction mixture was diluted with water and extracted with EtOAc (3×100 mL). The combined organic layers were washed with water (2×100 mL). The organic layer was then dried with MgSO₄ and concentrated under reduced pressure. The crude product was purified by flash chromatography (acetonitrile/MeOH (6:1)/DCM from 5% to 20%) to afford N-(3-(3-(6-ethyl-6H-thieno[2,3-b]pyrrole-5-carbonyl)-2-methyl-1H-indol-1-yl)propyl)methanesulfonamide (17.3 mg, 21%).

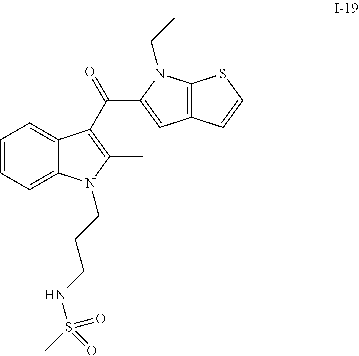 [SMILES: CCn1c(C(=O)c2c(C)n(CCCNS(C)(=O)=O)c3ccccc23)cc2ccsc21]

¹H NMR (CDCl₃/400 MHz) δ 7.74 (d, 1H), 7.32 (d, 1H), 7.22 (t, 1H), 7.11 (t, 1H), 6.95 (d, 1H), 6.92 (d, 1H), 6.88 (s, 1H), 4.58 (q, 2H), 4.35 (m, 1H), 4.28 (t, 2H), 3.22 (q, 2H), 2.95 (s, 3H), 2.63 (s, 3H), 2.09 (t, 2H), 1.57-1.52 (m, 3H).

The following compounds were prepared according to Example 12:

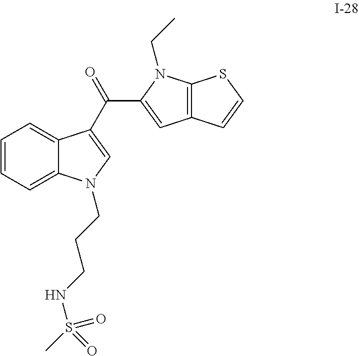 [SMILES: CCn1c(C(=O)c2cn(CCCNS(C)(=O)=O)c3ccccc23)cc2ccsc21]

¹H NMR (CDCl₃/400 MHz) δ 8.41 (dd, 1H), 7.81 (s, 1H), 7.41-7.39 (m, 1H), 7.36-7.31 (m, 2H), 7.04 (d, 1H), 7.00 (s, 1H), 6.94 (d, 1H), 4.53 (q, 2H), 4.34 (t, 2H), 3.15 (q, 2H), 2.93 (s, 3H), 2.18 (t, 2H), 1.53 (t, 3H).

### Example 13

### Saponifiation of Final Product

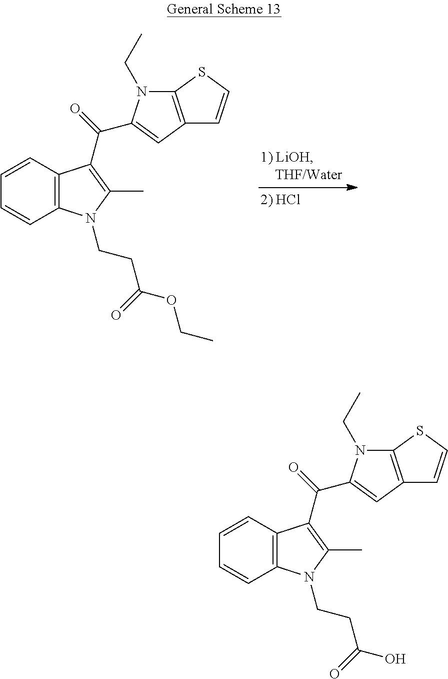 [SMILES: CCOC(=O)CCn1c(C)c(C(=O)c2cc3ccsc3n2CC)c2ccccc21 ; CCn1c(C(=O)c2c(C)n(CCC(=O)O)c3ccccc23)cc2ccsc21]

Preparation of Compound I-27

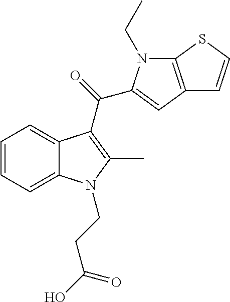 [SMILES: CCn1c(C(=O)c2c(C)n(CCC(=O)O)c3ccccc23)cc2ccsc21]

A microwave vial (20 ml) was charged with ethyl 3-(3-(6-ethyl-6H-thieno[2,3-b]pyrrole-5-carbonyl)-2-methyl-1H-indol-1-yl)propanoate (70 mg, 0.171 mmol) in THF. Lithium hydroxide ((2 ml) 41 mg, 17.1 mmol) in water (0.5 ml) was then added. The mixture was stirred at room temperature for 4 h. HCl (IN) was added to the mixture to achieve pH 2. A white precipitate crashed out. This solid was filtered and washed with water, dried on house vacuum over 2 days. Product was obtained as a white solid. 3-(3-(6-ethyl-6H-thieno[2,3-b]pyrrole-5-carbonyl)-2-methyl-1H-indol-1-yl)propanoic acid (50 mg, 77%). ¹H NMR (CDCl₃/400 MHz) δ 7.74 (d, 1H), 7.36 (d, 1H), 7.22 (t, 1H), 7.12 (t, 1H), 6.95 (d, 1H), 6.91 (d, 1H), 6.88 (s, 1H), 4.58 (q, 2H), 4.51 (t, 2H), 2.89 (t, 2H), 2.65 (s, 3H), 1.54 (t, 3H).

### Example 14

### Preparation of methyl 2-(5-methyl-4-(4H-thieno[3,2-b]pyrrole-5-carbonyl)-6H-thieno[2,3-b]pyrrol-6-yl)acetate (I-8)

### Step 1: Preparation of methyl 2-(5-methyl-6H-thieno[2,3-b]pyrrol-6-yl)acetate

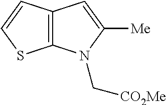 [SMILES: COC(=O)Cn1c(C)cc2ccsc21]

To a solution of 5-methyl-6H-thieno[2,3-b]pyrrole (1.09 g, 7.94 mmol) in acetonitrile (30 ml) was added methyl bromoacetate (2.20 mL, 23.8 mmol), potassium carbonate (1.65 g, 11.9 mmol), and potassium iodide (0.264 g, 1.59 mmol). The reaction was sealed and heated at 95° C. After 36 hours, the reaction was cooled to room temperature, extracted with DCM (3×40 mL), dried (sodium sulfate), filtered and concentrated to an orange oil. This was purified via column chromatography on silica gel using 10 to 35% ethyl acetate in hexanes over 40 minutes. Methyl 2-(5-methyl-6H-thieno[2,3-b]pyrrol-6-yl)acetate (981 mg, 4.69 mmol, 59.0% yield) was isolated as a residue which slowly solidified to a tan solid. ¹H NMR (400 MHz, CDCl₃) δ 6.93 (d, 1H), 6.78 (d, 1H), 6.19 (s, 1H), 3.77 (s, 3H), 2.32 (s, 3H).

### Step 2: preparation of 4H-thieno[3,2-b]pyrrole-5-carbonyl chloride

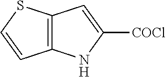 [SMILES: O=C(Cl)c1cc2sccc2[nH]1]

To a slurry of 4H-thieno[3,2-b]pyrrole-5-carboxylic acid (130 mg, 0.778 mmol) in chloroform (5 ml) was added phosphorus pentachloride (170 mg, 0.778 mmol). The reaction was heated to 70° C. for 15 minutes, and became brown and homogeneous. The solvent was then removed, and the tan solid 4H-thieno[3,2-b]pyrrole-5-carbonyl chloride (146 mg, 0.787 mmol, 100% yield) was used in the next step without purification.

### Step 3: Coupling

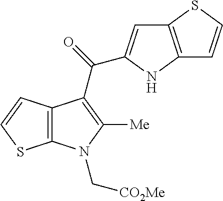 [SMILES: COC(=O)Cn1c(C)c(C(=O)c2cc3sccc3[nH]2)c2ccsc21]

To a 0° C. solution of methyl 2-(5-methyl-6H-thieno[2,3-b]pyrrol-6-yl)acetate (130 mg, 0.621 mmol) in dichloromethane (6 ml) was added 1M solution of diethylaluminum chloride in hexanes (0.932 ml, 0.932 mmol). The mixture was stirred at 0° C. for 30 minutes after which a solution of 4H-thieno[3,2-b]pyrrole-5-carbonyl chloride (138 mg, 0.745 mmol) in dichloromethane (4 ml) was added. The reaction was stirred at 0° C. for two hours then slowly allowed to warm to room temperature and stirred for 12 hours. The reaction mixture was poured directly over ice-cold 1N HCl solution (15 mL), extracted with dichloromethane (3×40 mL), dried (sodium sulfate), filtered and concentrated to a residue. This was purified on silica gel chromatography using 10 to 70% ethyl acetate in hexanes (over 37 minutes). Methyl 2-(5-methyl-4-(4H-thieno[3,2-b]pyrrole-5-carbonyl)-6H-thieno[2,3-b]pyrrol-6-yl)acetate (85.1 mg, 0.237 mmol, 38% yield) was isolated as a solid. ¹H NMR (400 MHz, CDCl₃) δ 9.36 (brs, 1H), 7.37 (d, 1H), 7.22 (d, 1H), 7.15 (d, 1H), 7.01 (d, 1H), 6.90 (d, 1H), 4.76 (s, 2H), 3.82 (s, 3H), 2.60 (s, 3H).

### Step 4

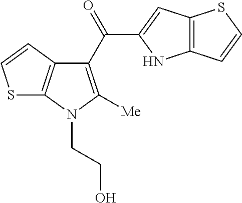 [SMILES: Cc1c(C(=O)c2cc3sccc3[nH]2)c2ccsc2n1CCO]

To a slurry of methyl 2-(5-methyl-4-(4H-thieno[3,2-b]pyrrole-5-carbonyl)-6H-thieno[2,3-b]pyrrol-6-yl)acetate (85.0 mg, 0.237 mmol) in 1,2-dichloroethane (3 ml) was added zinc iodide (114 mg, 0.356 mmol) followed by sodium cyanoborohydride (112 mg, 1.779 mmol). The reaction was sonicated for 30 minutes. After 2 hours, all starting material remained.

Continued to sonicate for another 30 minutes, after which tetrahydrofuran (0.2 ml) was added. The solubility of the reaction improved greatly. Still, no reaction was observed. Sodium borohydride (67.3 mg, 1.78 mmol) was then added to the reaction. After 6 hours, no reaction had taken place. Methanol (1 ml) was then added, and the reaction was heated to 75° C. The reaction ceased to progress after 67% conversion as monitored by LC/MS. The reaction was then quenched with water and extracted with dichloromethane (3×30 mL), dried (sodium sulfate), filtered and concentrated. Purification by column chromatography using silica gel (1 to 7% methanol in dichloromethane) afforded (6-(2-hydroxyethyl)-5-methyl-6H-thieno[2,3-b]pyrrol-4-yl)(4H-thieno[3,2-b]pyrrol-5-yl)methanone (10.7 mg, 0.0320 mmol, 14% yield). ¹H NMR (400 MHz, CD3OD) δ 7.44 (d, 1H), 7.09 (d, 1H), 7.02 (dd, 1H), 7.00 (d, 1H), 6.96 (d, 1H), 4.19 (t, 2H), 3.93 (t, 2H), 2.63 (s, 3H).

### Example 15

### Preparation of (6-(2-hydroxyethyl)-5-methyl-6H-thieno[2,3-b]pyrrol-4-yl)(quinolin-2-yl)methanone (1-9)

### Step 1: preparation of quinoline-2-carbonyl chloride

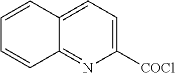 [SMILES: O=C(Cl)c1ccc2ccccc2n1]

Thionyl chloride (1.264 ml, 17.32 mmol) was added to a screwcap vial containing quinoline-2-carboxylic acid (1 g, 5.77 mmol). The reaction was heated at 60° C. for 6 hours and became homogeneous and red. After 6 hours, the reaction was concentrated to afford quinoline-2-carbonyl chloride (1.2 g, 6.26 mmol). The product was used in the next step without further purification. 1H NMR (400 MHz, CDCl₃) δ (ppm): 8.39 (d, 2H), 8.17 (d, 1H), 7.95 (dd, 1H), 7.86 (m, 1H), 7.75 (m, 1H).

### Step 2: Methyl 2-(5-methyl-4-(quinoline-2-carbonyl)-6H-thieno[2,3-b]pyrrol-6-yl)acetate

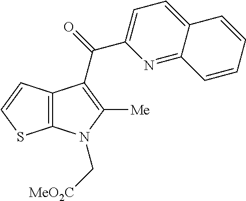 [SMILES: COC(=O)Cn1c(C)c(C(=O)c2ccc3ccccc3n2)c2ccsc21]

To a solution of methyl 2-(5-methyl-6H-thieno[2,3-b]pyrrol-6-yl)acetate (250 mg, 1.20 mmol) in dichloromethane (6 ml) was added diethylaluminum chloride (1.79 mL, 1.79 mmol). After 30 minutes, at 0° C., a solution of quinoline-2-carbonyl chloride (275 mg, 1.43 mmol) in dichloromethane (4 mL) was added. The reaction was allowed to warm to room temperature over 2 hours and stirring continued for 14 hours, after which the reaction was poured over iced 1N HCl, and extracted with dichloromethane (3×40 mL). The aqueous layer was basified and re-extracted with dichloromethane (2×40 mL), dried (sodium sulfate), filtered and concentrated. The crude mixture was purified by column chromatography using silica gel (10 to 100% ethyl acetate (with 1% triethylamine) in hexanes). Methyl 2-(5-methyl-4-(quinoline-2-carbonyl)-6H-thieno[2,3-b]pyrrol-6-yl)acetate (149 mg, 0.409 mmol, 34.2% yield) was isolated as a tan solid. Several fractions were discarded due to a close-running impurity. ¹H NMR (400 MHz, CD3OD) δ 8.53 (d, 1H), 8.03-8.10 (m, 2H), 7.82-7.87 (m, 2H), 7.69-7.74 (m, 1H), 6.84 (d, 1H), 6.22 (d, 1H), 5.01 (s, 2H), 3.79 (s, 3H), 2.61 (s, 3H).

### Step 3: (6-(2-hydroxyethyl)-5-methyl-6H-thieno[2,3-b]pyrrol-4-yl)(quinolin-2-yl)methanone

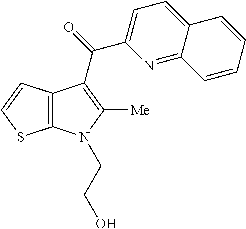 [SMILES: Cc1c(C(=O)c2ccc3ccccc3n2)c2ccsc2n1CCO]

To a solution of methyl 2-(5-methyl-4-(quinoline-2-carbonyl)-6H-thieno[2,3-b]pyrrol-6-yl)acetate (78.9 mg, 0.217 mmol) in methanol (8 ml) was added sodium borohydride (84.0 mg, 2.17 mmol). The reaction was sealed and heated at 85° C. After 16 hours, the reaction was allowed to cool down to room temperature, and then quenched with saturated ammonium chloride solution (15 mL), extracted with dichloromethane (3×35 mL), dried (sodium sulfate), filtered and concentrated. Purification by silica gel chromatography using 1 to 7% methanol in dichloromethane afforded (6-(2-hydroxyethyl)-5-methyl-6H-thieno[2,3-b]pyrrol-4-yl)(quinolin-2-yl)methanone (9.5 mg, 0.028 mmol, 13% yield). ¹H NMR (400 MHz, CD3OD) δ 8.53 (d, 1H), 8.04-8.08 (m, 2H), 7.82-7.87 (m, 2H), 7.69-7.74 (m, 1H), 6.84 (d, 1H), 6.16 (d, 1H), 5.01 (s, 2H), 4.23 (t, 2H), 3.93 (t, 2H), 2.69 (s, 3H).

### Example 16

### Preparation of Compound I-24

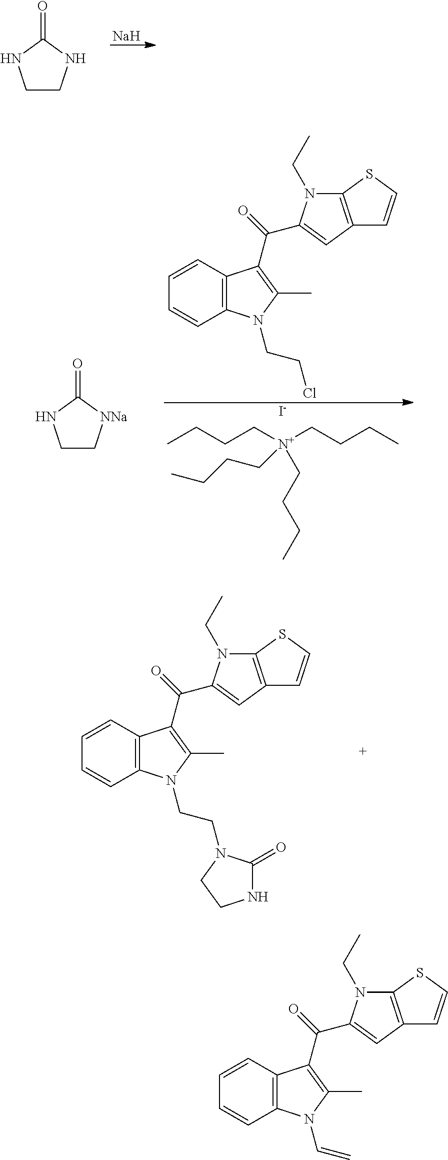

To a round bottomed flask, charged with sodium hydride (70.2 mg, 2.92 mmol) and imidazolidin-2-one (285 mg, 3.31 mmol) was added THF (2 mL). The mixture gave off gas and was heated at 50° C. for 15 minutes (after which time gas no longer evolved). A suspension resulted. To the mixture was added (1-(2-chloroethyl)-2-methyl-1H-indol-3-yl)(6-ethyl-6H-thieno[2,3-b]pyrrol-5-yl)methanone (72.3 mg, 0.195 mmol) as a solution in THF (2 mL). Subsequently, tetrabutylammonium iodide (108 mg, 0.292 mmol) was added. Heating was resumed at 50° C. for 19 h. The reaction mixture was diluted with THF to 3 mL and DMF (3 mL) was added. Heated at 70° C. in a sealed vial for an additional 7 hr. After a brief workup with water and DCM, drying, and concentrating, an oil was obtained (DMF still present). Elimination product is the only product observed. The crude product was added to a 40 g ISCO silica gel column and was purified with a gradient of 0% to 30% EtOAc/hexanes. The NMR of the product was consistent with the olefin. The product is a colorless foam (some remains an oil). ¹H NMR (CDCl₃/400 MHz) δ 7.73 (d, 1H), 7.58 (d, 1H), 7.23 (t, 1H), 7.14 (t, 1H), 7.02 (q, 1H), 6.96 (d, 1H), 6.93-6.92 (m, 2H), 5.56 (d, 1H), 5.37 (d, 1H), 4.60 (q, 2H), 2.61 (s, 3H), 1.55 (t, 3H).

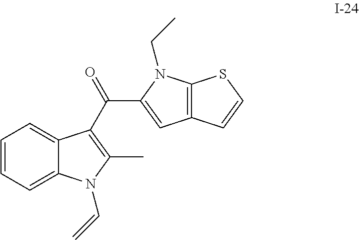 [SMILES: C=Cn1c(C)c(C(=O)c2cc3ccsc3n2CC)c2ccccc21]

¹H NMR (CDCl₃/400 MHz) 7.73 (d, J=8.0 Hz, 1H), 7.58 (d, J=8.0 Hz, 1H), 7.23 (t, J=7.2 Hz, 1H), 7.14 (t, J=7.6 Hz, 1H), 7.02 (q, J=4.8 Hz, 1H), 6.96 (d, J=5.2 Hz, 1H), 6.93-6.92 (m, 2H), 5.56 (d, J=15.2 Hz, 1H), 5.37 (d, J=8.8 Hz, 1H), 4.60 (q, J=6.8 Hz, 2H), 2.61 (s, 3H), 1.55 (t, J=7.2 Hz, 3H).

### Example 17

### Biological Activity Measurements

A. CB1 and CB2 Clones

cDNA expression clones for human CB 1 (hCB1, Genbank Accession No. AY225225) and human CB2 (hCB2, Genbank Accession No. AY242132) expressed in vector pcDNA3.1+ were purchased from UMR cDNA Resource Center, Rolla, Mo. (Clone ID CNR01L000 for hCB1; CNR0200000 for hCB2).

B. Stable and Transient Transfection

Stable, HEK-293-derived cell lines that recombinantly express hCB1 or hCB2 were established. In brief, the clone hCB1 (CNR1L) or hCB2 (CNR2) was transfected into human embryonic kidney cells (HEK-293) using Lipofectamine 2000 (Gibco, Cat#11668-019) according to the manufacturer's protocol. Transfected clones were isolated by single colony purification and clones were screened for receptor expression using a whole cell, ³H—CP 55,940 radioligand binding assay. HEK-293 stable cells were maintained in Dulbecco's modified Eagles medium (DMEM) containing 10% fetal bovine serum, 2 mM L-glutamine and 0.5 mg/mL G-418.

C. Human CB 1 and CB2 Cannabinoid Receptor Radioligand Binding Assays

i) Preparation of Membrane Suspensions

Membranes were isolated from transfected cells as follows. Monolayers of cultured cells were washed twice with phosphate-buffered saline (PBS). Cells were scraped into 20 mM HEPES, pH 7.4, 10 mM EDTA containing complete cocktail protease inhibitors (Roche, Catalog #11 697 498 001), and were homogenized by an electric-powered mechanical probe homogenizer (Omni GLH; probe G7-195S) for 40 seconds at 7000 rpm. Homogenates were centrifuged 10 minutes at 1000×g at 4° C. The supernatant was collected and was centrifuged for 1 hour at 40,000×g. The supernatant was then decanted and the resulting pellet was re-suspended in 20 mM HEPES, pH 7.4, 5 mM MgCl₂, 1 mM EDTA, 10% sucrose with complete cocktail protease inhibitors. Protein concentration of membrane suspensions were measured by Bradford Protein Assay using bovine serum albumin as the standard (BioRad catalog #500-0006). Protein concentrations of membrane suspensions were adjusted with the final buffer in the range of 5 to 10 mg/mL and were stored at −80° C. until further use.

ii) Radioligand Binding Assays

Radioligand binding assays were performed by incubating membranes (2-10 μg protein) prepared from HEK-293 cells expressing recombinant human cannabinoid receptors, CB 1 or CB2, at room temperature with 0.5 nM cannabinoid receptor agonist, [³H]-CP 55,940 (Perkin Elmer, catalog #NET 1051) in 0.2 mL of binding buffer (50 mM Tris-HCl, pH 7.5, 5 mM MgCl₂, 2.5 mM EDTA) and 0.1% fatty acid free bovine serum albumin (Sigma Cat. #A0821) for 90 minutes. A rapid filtration technique using Millipore FB filter plates (Catalog #MADVNOB) and filtration apparatus (Millipore system Catalog MAVM0960R) with vacuum aspiration was used to harvest and rinse labeled membranes (8 times with 0.2 mL of chilled binding buffer). The radioactivity bound to the filters was counted with 0.05 mL of liquid scintillant (UltraGold MV, PerkinElmer catalog #6013159) in a scintillation counter (Perkin Elmer Microbeta instrument). Nonspecific binding was determined in the presence of unlabeled 1 μM CP 55,940 (Sigma Aldrich, catalog #C1112). Binding data were analyzed using GraphPad Prism (GraphPad Software, Inc. San Diego, Calif.).

D. Human and Rodent CB1 and CB2 Receptor Functional Assays

Functional assays which monitor G-protein coupled receptor or downstream cellular responses can be used to characterize potential CB 1 receptor and/or CB2 receptor agonist or antagonist activities. Direct activation (or inhibition of activation) can be monitored using a GTPγS assay (membrane-based assay) or cAMP assay (whole cell-based assay).

i) GTPγS Assay

³⁵S GTPγS binding assays were performed by incubating recombinant cell membranes prepared above (5 μg) in the presence of scintillation proximity assay beads (SPA beads, Catalog #RPNQ0252 GE Healthcare, Buckinghamshire, England) in GTPγS binding buffer [50 mM HEPES (pH 7.4), 100 mM NaCl, 5 mM MgCl₂, 0.001% saponin (Sigma catalog #S4521)] supplemented with 20 uM GDP (Sigma catalog #G7127) in the presence or absence of test compound. The reaction was carried out in 96-well microplates with 0.1 nM [³⁵S]GTPγS (specific activity=1250 Ci/mmol; Perkin Elmer catalog #NEG030X250UC) in a final volume of 100 μL. After a 90 min room temperature incubation, the reaction was analyzed using a scintillation counter (Perkin Elmer Microbeta instrument). Binding data were analyzed using GraphPad Prism.

ii) cAMP Assay

cAMP assays were performed in HEK-293 cells stably expressing human CB 1 or CB2 receptors. For cannabinoid receptor functional assays measuring agonist effects on cellular cAMP levels, monolayers of cultured cells were harvested with enzyme-free PBS-based cell dissociation buffer (Gibco, Cat#13151-04). Cells suspensions were centrifuged and the cells were washed once with PBS, were centrifuged again, and the cells were resuspended in HBSS (Hank's Balanced Salt Solution, Cellgro, Cat #21-022-CV) solution containing 10 mM HEPES and 0.1% fatty acid free BSA (Sigma, Cat #A0281). Cell suspensions were prepared at 1,500,000 cells per mL. Stock solutions of test substances (10 mM) in DMSO were diluted to 1 mM using 30% DMSO as diluent. Test substances of solutions (1 mM) were further diluted down to 3× of final assay concentrations in the above HBSS buffer containing 0.1% BSA in the presence of 90 uM forskolin (Sigma Cat#F6886). To perform the assay, 20 μL of cell suspension (1,500,000 cells/mL) were added to each well in 96 well plate and treated with 10 μL test substance solution diluted as described above. Cells and compounds were incubated at 37° C. for 30 minutes. Cells were lysed and cAMP concentration was measured using DiscoveRx-XS+ cAMP assay kit (DiscoveRx Corporation Ltd., Fremont, Calif., USA, Cat #90-0075-03), following the manufacturer's protocol. GraphPad Prism software was used to calculate EC₅₀ values using sigmoidal dose response curve fitting. The maximal amount of cAMP produced by forskolin was defined as 100%. CB1 or CB2 agonists reduced forskolin-stimulated cAMP signaling. The EC₅₀ value of an agonist compound was defined as the concentration at which 50% of the forskolin-stimulated cAMP synthesis was inhibited.

E. Animal Model for Assessing Anti-Inflammatory Activity: Complete Freund's Adjuvant (CFA) Induced Inflammatory Pain

In this rat model of inflammatory pain, 100 μL of Complete Freund's Adjuvant (CFA) diluted 1:1 with phosphate buffered saline was injected into the subplantar region of the right hind paw on Day 1. On day 3, test compounds were administered orally and rats were assessed for their reaction to a mechanical stimuli applied via an Analgesy® meter. Injection of CFA increased the rats' reactivity to painful stimuli and this was reflected in a decrease in the amount of pressure they could tolerate prior to withdrawing their paw from the apparatus (hyperalgesia). An anti-hyperalgesic activity of the test compound was denoted by an increase in the amount of pressure they can tolerate prior to withdrawing their paw from the apparatus. The mean±SEM for each treatment group was determined and a Dunnett test was applied for comparison between vehicle and treated groups. Differences were considered significant at P<0.05. (see Bertorelli et al., 1999, Brit. J. Pharmacol. 128:1252).

F. Animal Model for Assessing Analgesic Activity: Phenylbenzoquinone-Induced (PBQ) Writhing Model

This model is described by Siegmund et al. (1957) Proc. Soc. Exp. Bio. Med. 95:729. Briefly, one hour after oral or intraperitoneal dosing with a test compound, morphine or vehicle, 0.02% phenylbenzoquinone (PBQ) solution (12.5 mL/kg) was injected by intraperitoneal route into the mouse. The number of stretches and writhings were recorded from the 5th to the 10th minutes post PBQ injection, and were also counted between the 35^{th} and 40^{th} minutes and between the 60^{th} and 65^{th} minutes post injection to provide a kinetic assessment. The results were expressed as the number of stretches and writhings (mean±SEM) and the percentage of variation of the nociceptive threshold calculated from the mean value of the vehicle-treated group. The statistical significance of any differences between the treated groups and the control group was determined by a Dunnett's test using the residual variance after a one-way analysis of variance (P<0.05) using SigmaStat Software.

### Example 18

### Biological Data for Compounds of the Invention

Data for compounds of the invention are summarized in Table 1 and Table 2.

TABLE 1
hCB2/hCB1 Activity.

| | | hCB1 EC50 | hCB1 EC50 | | | hCB2 EC50 | |
|---|---|---|---|---|---|---|---|
| | hCB1 EC50 | (cAMP ag, | (cAMP ag, | hCB1 IC50 | hCB2 EC50 | (cAMP ag, | hCB2 IC50 |
| Sample | (GTPyS ag, | nM HEK | nM, HEK | (CP displ, | (GTPyS ag, | nM, HEK | (CP displ, |
| ID | nM, HEK) | (24-well)) | (96-well)) | nM, HEK) | nM, HEK) | (96-well)) | nM, HEK) |
| I-1 | ND | ND | ND | B | A | ND | A |
| I-10 | NS | ND | ND | C | ND | ND | B |
| I-11 | B | ND | ND | A | A | A | A |
| I-12 | ND | ND | ND | C | A | ND | B |
| I-13 | NS | ND | ND | D | NS | ND | D |
| I-14 | NS | ND | NS | B | B | B | B |
| I-16 | A | ND | A | A | A | A | A |
| I-17 | ND | ND | B | B | A | A | A |
| I-18 | ND | ND | ND | B | A | A | A |
| I-19 | NS | ND | NS | C | A | A | A |
| I-2 | ND | ND | ND | C | B | ND | B |
| I-20 | ND | ND | ND | C | B | B | C |
| I-21 | NS | ND | B | D | B | C | B |
| I-22 | NS | ND | NS | C | B | A | B |
| I-23 | NS | ND | NS | C | B | A | A |
| I-24 | NS | ND | ND | B/C | B | A | B |
| I-25 | NS | ND | C | D | A | A | A |
| I-26 | NS | ND | NS | D | NS | A | B |
| I-27 | NS | ND | ND | D | ND | B | B |
| I-28 | C | ND | B | B | A | A | A |
| I-29 | B | ND | A | A | A | A | A |
| I-3 | ND | ND | ND | D | A | ND | C |
| I-30 | NS | ND | NS | D | NS | ND | C |
| I-31 | NS | ND | NS | C | B | A | B |
| I-32 | ND | ND | B | B | A | A | A |
| I-34 | B | ND | C | C | A | A | A |
| I-36 | ND | ND | B | B | A | A | A |
| I-37 | NS | ND | ND | D | B | A | B |
| I-38 | NS | ND | NS | D | ND | NS | C |
| I-39 | NS | ND | NS | D | NS | NS | D |
| I-4 | ND | ND | ND | D | C | ND | C |
| I-40 | NS | ND | NS | D | NS | NS | D |
| I-41 | A | ND | A | A | A | A | A |
| I-42 | B | ND | B | A | A | A | A |
| I-43 | NS | ND | ND | D | B | NS | C |
| I-44 | NS | ND | NS | D | NS | ND | D |
| I-45 | B | ND | B | A | A | A | A |
| I-45 | B | ND | B | B | A | A | A |
| I-46 | ND | ND | ND | C | B | B | B |
| I-47 | NS | ND | B | B | B | A | A |
| I-48 | ND | ND | ND | C | B | B | B |
| I-49 | NS | ND | NS | C | A | A | A |
| I-5 | NS | ND | ND | B | A | ND | A |
| I-50 | NS | ND | ND | C | A | A | A |
| I-51 | NS | ND | NS | D | ND | B | B |
| I-52 | NS | ND | ND | C | B | B | B |
| I-53 | NS | ND | NS | D | B | B | B |
| I-54 | NS | ND | ND | C | A | A | A |
| I-55 | NS | ND | ND | D | B | A | B |
| I-56 | NS | ND | ND | C | A | A | A |
| I-57 | B | ND | B | B | A | A | A |
| I-58 | NS | ND | A | B | A | A | A |
| I-59 | ND | ND | ND | B | B | A | A |
| I-6 | ND | ND | NS | D | NS | ND | D |
| I-60 | B | ND | A | A | A | A | A |
| I-61 | NS | ND | ND | B | NS | B | A |
| I-62 | B | ND | A | A | A | A | A |
| I-64 | NS | ND | ND | D | B | B | B |
| I-65 | NS | ND | ND | C | A | A | A |
| I-66 | B | ND | A | B | A | A | A |
| I-67 | NS | ND | NS | D | NS | B | B |
| I-68 | NS | ND | NS | D | NS | ND | C |
| I-69 | NS | ND | NS | C | A | A | A |
| I-7 | ND | ND | ND | C | NS | ND | B |
| I-70 | B | ND | B | B | A | A | A |
| I-71 | ND | ND | ND | D | ND | ND | C |
| I-72 | NS | ND | NS | B | B | A | B |
| I-73 | ND | ND | B | B | A | A | A |
| I-74 | ND | ND | NS | C | B | B | B |
| I-75 | ND | ND | ND | D | NS | ND | C |
| I-76 | ND | ND | NS | D | B | B | B |
| I-77 | NS | NS | NS | C | A | A | A |
| I-78 | NS | NS | NS | C | A | A | A |
| I-79 | ND | ND | ND | B | A | A | A |
| I-80 | NS | ND | C | B | A | A | A |
| I-81 | ND | ND | NS | C | B | B | B |
| I-82 | ND | ND | ND | C | B | B | B |
| I-83 | B | A | A | NS | ND | A | ND |
| I-84 | D | ND | A | NS | ND | ND | ND |
| I-85 | NS | ND | ND | C | A | A | A |
| I-86 | B | ND | B | A | A | A | A |
| I-87 | NS | ND | ND | C | A | A | A |
| I-35 | NS | ND | ND | B | A | A | A |
| I-63 | ND | ND | ND | C | NS | NS | B |

A = Less than 100 nM; B = between 100 nM and 1 μM; C = between 1 μM and 10 μM; D = greater than 10 μM. NS means “Not Significant,” which means less than 30% agonist activity when compared to the positive control. ND means “Not Determined.”

TABLE 2
PBQ writhing model data

| Compound | | | Number of | Statistical |
|---|---|---|---|---|
| Number | Dose | Route | Writhes | Significance |
| I-54 | 0 | IP | 14.1 ± 2.38 | |
| | 30 | | 3.67 ± 1.42 | P < 0.01 |
| I-78 | 0 | IP | 15.6 ± 2.21 | |
| | 30 | | 2.3 ± 1.42 | P < 0.001 |
| I-78 | 0 | IP | 16.71 ± 1.37 | |
| | 1 | | 14.82 ± 1.66 | |
| | 3 | | 14.76 ± 1.79 | |
| | 10 | | 6.41 ± 1.43 | P < 0.01 |
| | 30 | | 1.42 ± 0.68 | P < 0.01 |
| | 100 | | 1.0 ± 0.88 | P < 0.01 |
| I-77 | 0 | PO | 23.55 ± 1.49 | |
| | 10 | | 18.6 ± 2.52 | |
| | 30 | | 11.56 ± 3.0 | P < 0.01 |
| | 100 | | 5.7 ± 2.41 | P < 0.001 |
| I-42 | 0 | PO | 18.0 ± 1.98 | |
| | 30 | | 3.89 ± 1.56 | P < 0.001 |
| I-19 | 0 | PO | 17.7 ± 2.76 | |
| | 30 | | 10.6 ± 1.5 | P < 0.05 |
| I-70 | 0 | PO | 17.7 ± 2.76 | |
| | 30 | | 4.3 ± 1.4 | P < 0.001 |
| I-87 | 0 | PO | 17.7 ± 2.76 | |
| | 30 | | 15.6 ± 3.32 | NS |
| I-62 | 0 | PO | 15.6 ± 2.6 | |
| | 30 | | 0.1 ± 0.1 | P < 0.001 |
| I-45 | 0 | PO | 15.6 ± 2.6 | |
| | 30 | | 1.9 ± 0.48 | P < 0.001 |
| I-34 | 0 | PO | 15.6 ± 2.6 | |
| | 30 | | 6.4 ± 1.78 | P < 0.01 |
| I-25 | 0 | PO | 23.2 ± 1.47 | |
| | 30 | | 21.2 ± 1.59 | NS |
| I-67 | 0 | IP | 15.6 ± 2.21 | |
| | 30 | | 8.4 ± 3.1 | NS |

1. A compound of formula I or a pharmaceutically acceptable salt thereof, 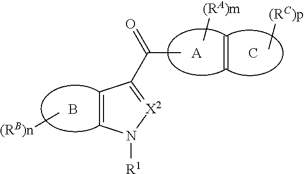
wherein
R¹ is -V-R⁸;
V is a single bond or a divalent linker between R⁸ and the nitrogen to which V is bonded, wherein said linker is a C₁₋₆ aliphatic; and wherein up to two saturated carbons of said C₁₋₆ aliphatic are optionally and independently replaced by —O—, —C(O)—, —C(S)—, —C(O)N(R)—, —N(R)C(O)—, —(R)NC(O)N(R)—, —C(O)O—, —OC(O)—, —N(R)—, —N(R)S(O)₂—, —S(O)₂N(R)—, N(R)S(O)₂N(R)— or —S(O)_{q}—;
each q is an integer independently selected from 0, 1 or 2;
each occurrence of R is independently selected from hydrogen, a C₁₋₄ alkyl, C₁₋₄haloalkyl, C₃₋₆ cycloalkyl or —C(O)(C₁₋₄ alkyl);
R⁸ is selected from hydrogen, halogen, —CN, —NO₂, phenyl, a 5-6-membered heteroaryl ring, a C₃₋₁₀ cycloaliphatic ring or a 3-10-membered heterocyclyl ring; wherein each said phenyl, heteroaryl, cycloaliphatic or heterocyclyl ring is optionally and independently substituted with up to 6 instances of R¹⁵;
each occurrence of R¹⁵ is independently selected from halogen, oxo, —NO₂, —CN, —OR¹⁶, —N(R¹⁶)₂, —C(O)OR¹⁶, —C(O)R¹⁶, —N(R′)C(O)R¹⁶, —C(O)N(R¹⁶)₂, —OC(O)R¹⁶, —SR¹⁶, —S(O)₄R¹⁶, —SO₂N(R¹⁶)₂, —S(O)R¹⁶, a C₁₋₆ aliphatic or a C₃₋₆ cycloaliphatic; wherein each of said C₁₋₆ aliphatic and C₃₋₆ cycloaliphatic groups is optionally and independently substituted by up to six instances of halogen, oxo, C₁₋₄ alkyl, C₁₋₄ haloalkyl, —CN, C₁₋₄ alkoxy, —N(R¹⁰)₂ or C₁₋₄ haloalkoxy;
each occurrence of R′ is independently selected from hydrogen, C₁₋₄ alkyl, C₁₋₄ haloalkyl, C₃₋₆ cycloalkyl or —C(O)(C₁₋₄ alkyl);
each occurrence of R¹⁶ is independently selected from hydrogen, a C₁-C₄ aliphatic, a C₁-C₄ haloaliphatic, a C₃₋₇ cycloaliphatic or a 3-7-membered heterocyclyl; or two R¹⁶ groups attached to the same nitrogen atom, together with the nitrogen atom to which they are attached, form a 3-7-membered heterocycle; wherein each instance of R¹⁶ and each cycle formed by two R¹⁶ groups is optionally and independently substituted by up to 6 instances of halogen, —CN, oxo, C₁₋₄ alkyl, C₁₋₄ haloalkyl, —N(R¹⁰)₂, C₁₋₄ alkoxy or C₁₋₄ haloalkoxy;
each occurrence of R¹⁰ is independently selected from hydrogen or C₁₋₄ alkyl; or 2 instances of R¹⁰ attached to the same nitrogen atom, which, together with the nitrogen atom to which they are attached, form a 3-7-membered heterocyclic ring;
X² is selected from N or CR²;
R² is selected from hydrogen, halogen, —CN, —NO₂, —CO₂R¹⁷, —CO₂R¹⁷, —C(O)N(R¹⁷)₂, a C₁₋₆ alkyl, —O(C₁₋₄ alkyl) or a C₃₋₆ cycloaliphatic ring; wherein said alkyl, —O(alkyl) or cycloaliphatic ring is optionally and independently substituted with up to 6 instances of halogen, —CN, oxo, —O(C₁₋₂ alkyl) or —O(C₁₋₂ haloalkyl);
each R¹⁷ is independently selected from hydrogen, a C₁₋₄ alkyl, C₁₋₄ haloalkyl, C₃₋₆ cycloalkyl, phenyl or a 5-6-membered heteroaryl;
ring A is selected from a benzene ring or a 5-6-membered heteroaryl ring having 1-3 heteroatoms independently selected from N, O or S;
m is an integer selected from 0, 1, 2 or 3;
each occurrence of R^{A} is independently selected from halogen, —NO₂, —CN, oxo, —OR¹³, —SR¹³, —S(O)₂R¹³, —SO₂N(R¹³)₂, —N(R¹³)₂, —C(O)OR¹³, —C(O)R¹³, —N(R¹³)C(O)R¹³, —N(R¹³)S(O)₂R¹³, C(O)N(R¹³)₂, —OC(O)R¹³ or to C₁₋₄ aliphatic; wherein each of said aliphatic groups is optionally and independently substituted with up to our instances of
each occurrence of R¹³ is independently selected from hydrogen, a C₁-C₄ aliphatic, a C₃₋₇ cycloaliphatic or a 3-7-membered heterocyclyl; wherein each of said aliphatic, cycloaliphatic and heterocyclyl groups is independently and optionally substituted with up to 6 instances of R¹⁸; or two instances of R¹³ attached to the same nitrogen atom, together with the nitrogen atom to which they are attached, form a 3-7-membered heterocycle; wherein each of said heterocycle rings is optionally and independently substituted with up to 6 instances of R¹⁸; provided that when R^{A} is a substituent on a nitrogen atom it can also be one of the R¹ groups defined above as possible substituents on the pyrrole ring nitrogen;
ring C is benzene or a 5-6-membered heteroaryl ring having 1 to 3 heteroatoms independently selected from N, O or S;
ring C is fused to ring A;
p is an integer selected from 0, 1, 2 or 3;
each occurrence of R^{C} is independently selected from halogen, —NO₂, —CN, oxo, —OR¹⁴, —SR¹⁴, —S(O)₂R¹⁴, —SO₂N(R¹⁴)₂, —N(R¹⁴)₂, —C(O)OR¹⁴, —C(O)R¹⁴, —N(R¹⁴)C(O)R¹⁴, —N(R¹⁴)S(O)₂R¹⁴, —C(O)N(R¹⁴)₂, —OC(O)R¹⁴ or a C₁₋₄ aliphatic; wherein each of said aliphatic groups is optionally and independently substituted with up to four instances of R¹⁸; or two instances of R^{C} attached to two vicinal ring C atom(s), together with the ring atom(s) to which they are attached, form a 3-7-membered heterocycle or C₃₋₇ cycloaliphatic; wherein said cycloaliphatic or heterocyclic rings are independently and optionally substituted with up to 6 instances of halogen, —CN, —OH, C₁₋₂ alkyl, C₁₋₂ haloalkyl, C₁₋₂ alkoxy or C₁₋₂ haloalkoxy;
provided that at least one of the bicyclic system formed by ring B and the fused pyrrole or pyrazole moiety, or the bicyclic system formed by rings A and C, consists of two fused 5-membered rings;
each R¹⁴ is independently selected from hydrogen, a C₁₋₄ aliphatic, a C₃₋₇ cycloaliphatic or a 3-7-membered heterocyclyl; wherein each of said aliphatic, cycloaliphatic and heterocyclyl groups is independently and optionally substituted with up to 6 instances of halogen; or two instances of R¹⁴ attached to the same or different atom(s), together with the atom(s) to which they are attached, form a 37-membered heterocycle; wherein said heterocycle is optionally and independently substituted with up to 6 instances of halogen, —CN, —OH, C₁₋₂ alkyl, C₁₋₂ haloalkyl, C₁₋₂ alkoxy or C₁₋₂ haloalkoxy;
each occurrence of R¹⁸ is independently selected from halogen, —OR¹⁹, —SR¹⁹, —CN, —OCOR¹⁹, —CO₂R¹⁹, —C(O)N(R¹⁹)₂, —N(R¹⁹)C(O)R¹⁹, —N(R¹⁹)₂, a C₁₋₄ aliphatic, a C₁₋₄ haloaliphatic, a C₃₋₆, cycloaliphatic or a 3-6-membered heterocyclyl; wherein each of said cycloaliphatic and heterocyclyl rings is optionally and independently substituted with up to 6 instances of halogen, —CN, —OH, C₁₋₂ alkyl, C₁₋₂ haloalkyl, C₁₋₂ alkoxy or C₁₋₂ haloalkoxy;
each occurrence of R¹⁹ is independently selected from hydrogen, a C₁₋₄ aliphatic or a C₁₋₄ haloaliphatic;
ring B is benzene, a 5-6-membered heteroaryl ring containing up to 3 heteroatoms selected from N, O and S, or a 5-8-membered carbocyclic or heterocyclic ring; wherein said benzene, heteroaryl, carbocyclic or heterocyclic ring is optionally and independently substituted with up to 6 instances of halogen, —CN, —OH, C₁₋₂ alkyl, C₁₋₂ haloalkyl, C₁₋₂ alkoxy or C₁₋₂ haloalkoxy;
n is an integer selected from 0, 1, 2 or 3;
each occurrence of R^{B} is independently selected from halogen, —CN, —NO₂, oxo, —C(O)NR^{X}, —C(O)OR^{X}, a C₁₋₄ aliphatic or a C₃₋₆ cycloaliphatic; wherein each of said aliphatic and cycloaliphatic groups is independently and optionally substituted with up to 6 instances of halogen, —CN, —OH, oxo, —O(C₁₋₂ alkyl), —O(C₁₋₂ haloalkyl), alkyl or —C₁₋₂ haloalkyl; and
each R_{X} is independently selected from hydrogen, a C₁₋₄ alkyl, C₁₋₄ haloalkyl, C₃₋₆ cycloalkyl, phenyl or a 5-6-membered heteroaryl.

2. The compound according to claim 1, wherein V is a bond or a C₁₋₆ alkylene linker, wherein up to two methylene units of said alkylene linker are optionally and independently replaced by —O—, —C(O)—, —C(O)N(R)—, —N(R)C(O)—, —C(O)O—, —N(R)—, —N(R)S(O)₂—, —S(O)₂N(R)— or —S(O)₂—.

3. The compound according to claim 1, wherein R⁸ is selected from hydrogen, halogen, a C₃₋₁₀ cycloaliphatic group or a 3-10-membered heterocyclyl ring: wherein said cycloaliphatic or heterocyclyl ring is optionally and independently substituted with up to 6 instances of R¹⁵.

4. The compound according to claim 1, wherein R¹ is hydrogen.

5. The compound according to claim 1, wherein R¹ is selected from the group consisting of:

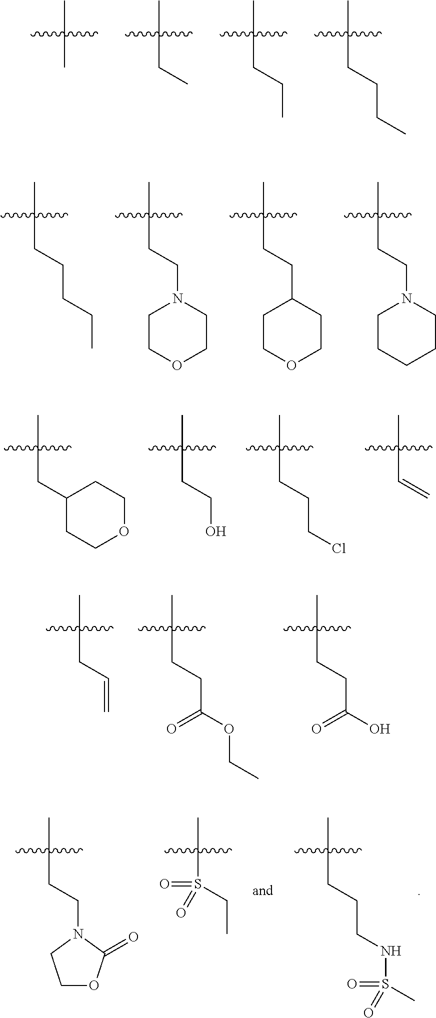

6. The compound according to claim 1, wherein Ring B is benzene, or a 5-6-membered heteroaryl ring containing up to 3 heteroatoms selected from N, O and S, or a 5-8-membered carbocyclic or heterocyclic ring; wherein said benzene, heteroaryl, carbocyclic or heterocyclic ring is optionally and independently substituted with up to 6 instances of halogen, —OH, C₁₋₂ alkyl or C₁₋₂ alkoxy.

7. The compound according to claim 6, wherein Ring B is benzene, optionally and independently substituted with up to 3 instances of halogen, —OH, C₁₋₂ alkyl or C₁₋₂ alkoxy.

8. The compound according to claim 7, wherein (Ring B)-(R^{B})n is 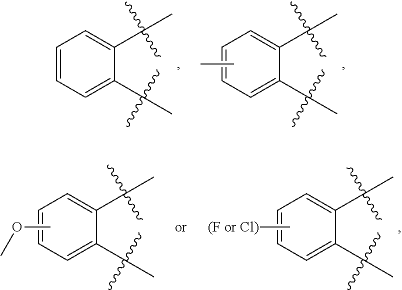
wherein the two broken lines represent the two points of attachment to the pyrrole or pyrazole ring.

9. The compound according to claim 6, wherein Ring B is a 5-6-membered heteroaryl ring containing up to 3 heteroatoms selected from N, O and S, and is optionally and independently substituted with up to 3 instances of halogen, —OH, C₁₋₂ alkyl or C₁₋₂ alkoxy.

10. The compound according to claim 9, wherein Ring B is pyridine, optionally and independently substituted with up to 3 instances of halogen, —OH, C₁₋₂ alkyl or C₁₋₂ alkoxy.

11. The compound according to claim 10, wherein Ring B is 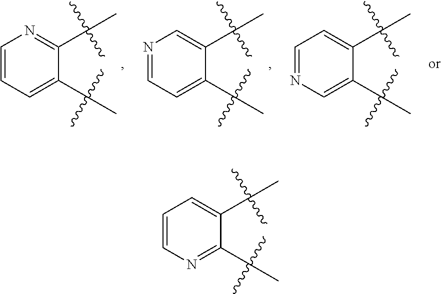 [SMILES: *c1cccnc1* ; *c1ccncc1* ; *c1ccncc1* ; *c1cccnc1*]
and is unsubstituted.

12. The compound according to claim 6, wherein Ring B is a thiophene, optionally and independently substituted with up to 3 instances of halogen, —OH, C₁₋₂ alkyl or C₁₋₂ alkoxy.

13. The compound according to claim 12, wherein Ring B is 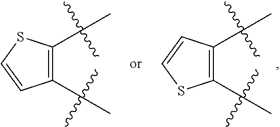 [SMILES: *c1ccsc1* ; *c1ccsc1*]
and is unsubstituted.

14. The compound according to claim 6, wherein Ring B is a 5-8-membered carbocyclic or heterocyclic ring, wherein said carbocyclic or heterocyclic ring is optionally and independently substituted with up to 3 instances of halogen, —OH, C₁₋₂ alkyl or C₁₋₂ alkoxy.

15. The compound according to claim 14, wherein (Ring B)-(R^{B})n is

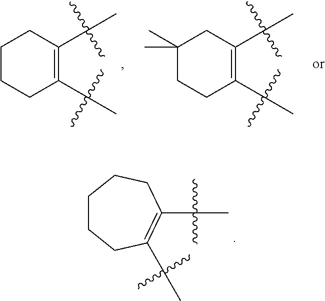

16. The compound according to claim 1, wherein X² is CR².

17. The compound according to claim 16, wherein R² hydrogen or C₁₋₆ alkyl.

18. The compound according to claim 17, wherein R² is hydrogen or methyl.

19. The compound according to claim 1, wherein Ring A is benzene, pyridine, thiophene or pyrrole, and Ring A is fused to Ring C.

20. The compound according to claim 1, wherein each instance of R^{A} is independently selected from halogen or a C₁₋₄ aliphatic; wherein each of said aliphatic groups is optionally and independently substituted with up to two instances of a C₃₋₆ cycloaliphatic or a 3-6-membered heterocyclyl.

21. The compound according to claim 1, wherein Ring C is benzene, pyridine, furan, thiophene or pyrrole, and Ring C is fused to Ring A.

22. The compound according to claim 1, wherein each instance of R^{C} is independently selected from halogen, or a C₁₋₄ aliphatic; wherein each of said aliphatic groups is optionally and independently substituted with up to two instances of a C₃₋₆ cycloaliphatic or a 3-6-membered heterocyclyl.

23. The compound according to claim 1, wherein the compound is selected from those depicted in the table below:

| | |
|---|---|
| 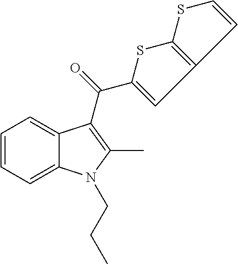 [SMILES: CCCn1c(C)c(C(=O)c2cc3ccsc3s2)c2ccccc21] | I-46 |
| 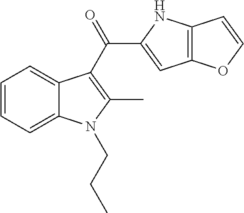 [SMILES: CCCn1c(C)c(C(=O)c2cc3occc3[nH]2)c2ccccc21] | I-71 |
| 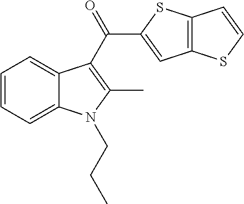 [SMILES: CCCn1c(C)c(C(=O)c2cc3sccc3s2)c2ccccc21] | I-20 |
| 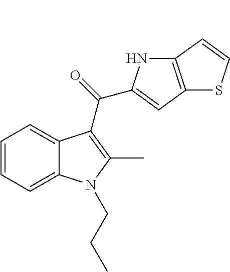 | I-81 |
| 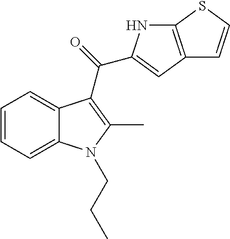 [SMILES: CCCn1c(C)c(C(=O)c2cc3ccsc3[nH]2)c2ccccc21] | I-74 |
| 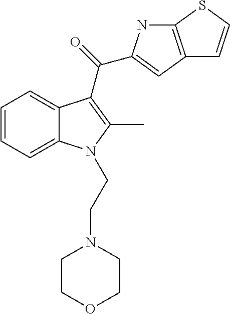 [SMILES: Cc1c(C(=O)c2cc3ccsc3[nH]2)c2ccccc2n1CCN1CCOCC1] | I-82 |
| 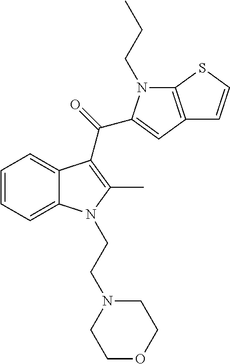 [SMILES: CCCn1c(C(=O)c2c(C)n(CCN3CCOCC3)c3ccccc23)cc2ccsc21] | I-73 |
| 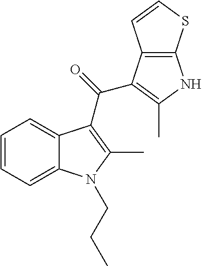 [SMILES: CCCn1c(C)c(C(=O)c2c(C)[nH]c3sccc23)c2ccccc21] | I-6 |
| 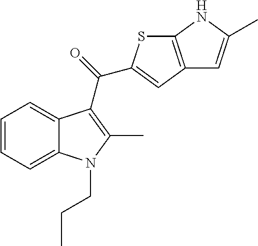 [SMILES: CCCn1c(C)c(C(=O)c2cc3cc(C)[nH]c3s2)c2ccccc21] | I-75 |
| 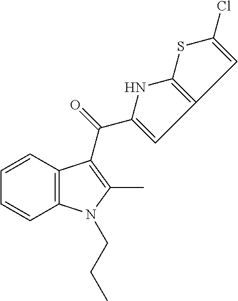 [SMILES: CCCn1c(C)c(C(=O)c2cc3cc(Cl)sc3[nH]2)c2ccccc21] | I-48 |
| 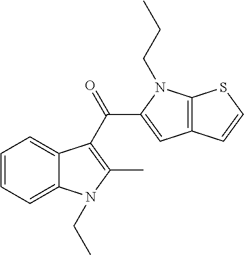 [SMILES: CCCn1c(C(=O)c2c(C)n(CC)c3ccccc23)cc2ccsc21] | I-50 |
| 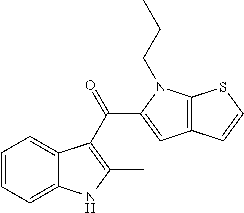 [SMILES: CCCn1c(C(=O)c2c(C)[nH]c3ccccc23)cc2ccsc21] | I-51 |
| 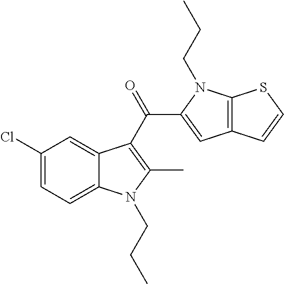 [SMILES: CCCn1c(C)c(C(=O)c2cc3ccsc3n2CCC)c2cc(Cl)ccc21] | I-52 |
| 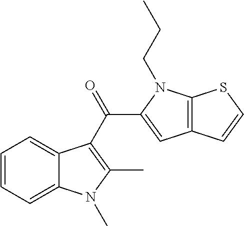 [SMILES: CCCn1c(C(=O)c2c(C)n(C)c3ccccc23)cc2ccsc21] | I-53 |
| 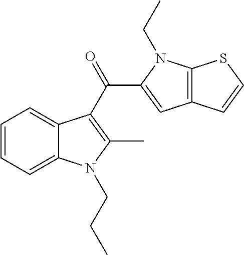 [SMILES: CCCn1c(C)c(C(=O)c2cc3ccsc3n2CC)c2ccccc21] | I-54 |
| 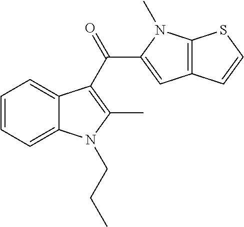 [SMILES: CCCn1c(C)c(C(=O)c2cc3ccsc3n2C)c2ccccc21] | I-55 |
| 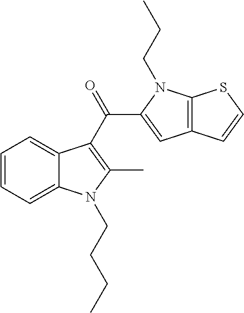 [SMILES: CCCCn1c(C)c(C(=O)c2cc3ccsc3n2CCC)c2ccccc21] | I-58 |
| 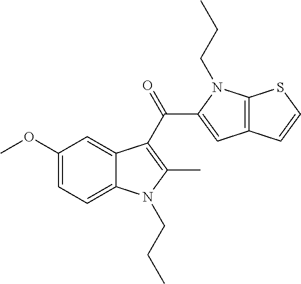 [SMILES: CCCn1c(C)c(C(=O)c2cc3ccsc3n2CCC)c2cc(OC)ccc21] | I-61 |
| 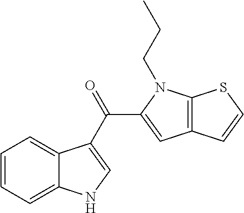 [SMILES: CCCn1c(C(=O)c2c[nH]c3ccccc23)cc2ccsc21] | I-64 |
| 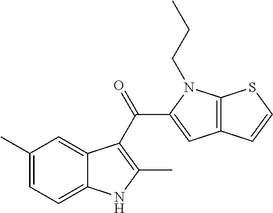 [SMILES: CCCn1c(C(=O)c2c(C)[nH]c3ccc(C)cc23)cc2ccsc21] | I-68 |
| 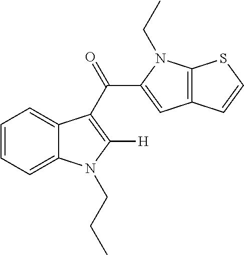 [SMILES: CCCn1cc(C(=O)c2cc3ccsc3n2CC)c2ccccc21] | I-70 |
| 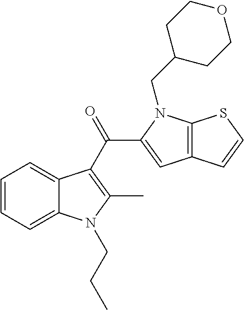 [SMILES: CCCn1c(C)c(C(=O)c2cc3ccsc3n2CC2CCOCC2)c2ccccc21] | I-72 |
| 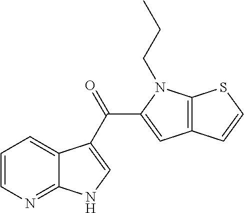 [SMILES: CCCn1c(C(=O)c2c[nH]c3ncccc23)cc2ccsc21] | I-43 |
| 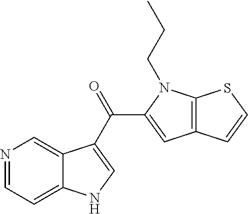 [SMILES: CCCn1c(C(=O)c2c[nH]c3ccncc23)cc2ccsc21] | I-44 |
| 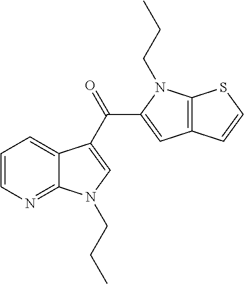 [SMILES: CCCn1cc(C(=O)c2cc3ccsc3n2CCC)c2cccnc21] | I-45 |
| 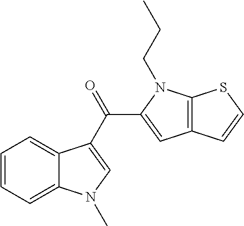 [SMILES: CCCn1c(C(=O)c2cn(C)c3ccccc23)cc2ccsc21] | I-47 |
| 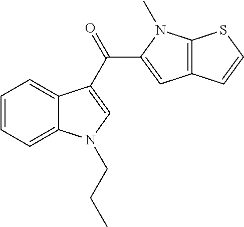 [SMILES: CCCn1cc(C(=O)c2cc3ccsc3n2C)c2ccccc21] | I-32 |
| 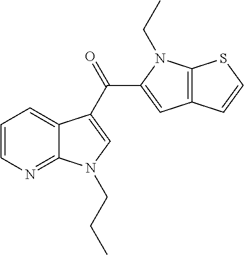 [SMILES: CCCn1cc(C(=O)c2cc3ccsc3n2CC)c2cccnc21] | I-34 |
| 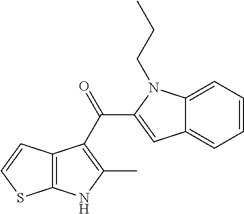 [SMILES: CCCn1c(C(=O)c2c(C)[nH]c3sccc23)cc2ccccc21] | I-13 |
| 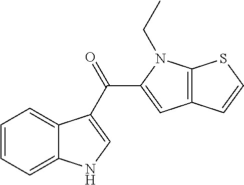 [SMILES: CCn1c(C(=O)c2c[nH]c3ccccc23)cc2ccsc21] | I-37 |
| 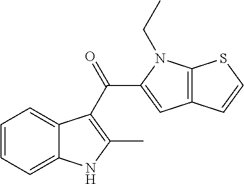 [SMILES: CCn1c(C(=O)c2c(C)[nH]c3ccccc23)cc2ccsc21] | I-38 |
| 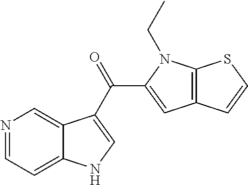 [SMILES: CCn1c(C(=O)c2c[nH]c3ccncc23)cc2ccsc21] | I-39 |
| 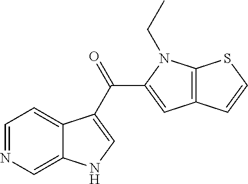 [SMILES: CCn1c(C(=O)c2c[nH]c3cnccc23)cc2ccsc21] | I-40 |
| 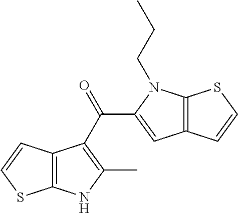 [SMILES: CCCn1c(C(=O)c2c(C)[nH]c3sccc23)cc2ccsc21] | I-84 |
| 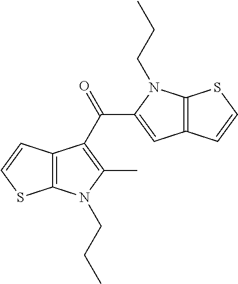 [SMILES: CCCn1c(C(=O)c2c(C)n(CCC)c3sccc23)cc2ccsc21] | I-83 |
| 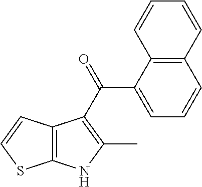 [SMILES: Cc1[nH]c2sccc2c1C(=O)c1cccc2ccccc12] | I-3 |
| 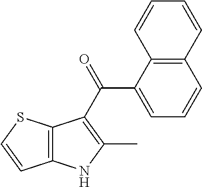 [SMILES: Cc1[nH]c2ccsc2c1C(=O)c1cccc2ccccc12] | I-4 |
| 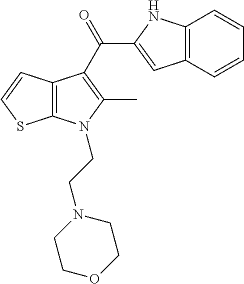 [SMILES: Cc1c(C(=O)c2cc3ccccc3[nH]2)c2ccsc2n1CCN1CCOCC1] | I-10 |
| 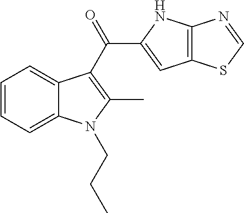 [SMILES: CCCn1c(C)c(C(=O)c2cc3scnc3[nH]2)c2ccccc21] | I-30 |
| 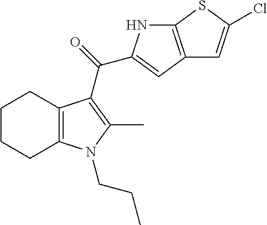 [SMILES: CCCn1c(C)c(C(=O)c2cc3cc(Cl)sc3[nH]2)c2c1CCCC2] | I-76 |
| 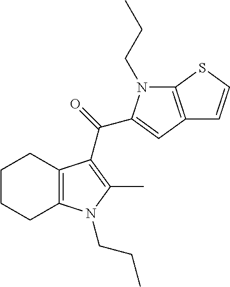 [SMILES: CCCn1c(C)c(C(=O)c2cc3ccsc3n2CCC)c2c1CCCC2] | I-77 |
| 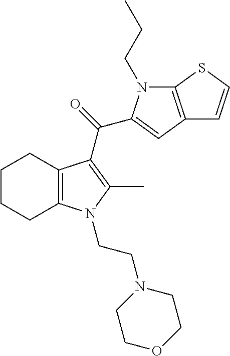 [SMILES: CCCn1c(C(=O)c2c3c(n(CCN4CCOCC4)c2C)CCCC3)cc2ccsc21] | I-79 |
| 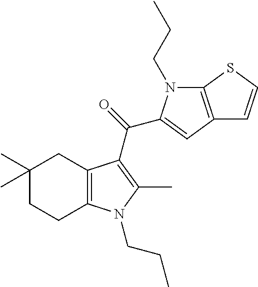 [SMILES: CCCn1c(C)c(C(=O)c2cc3ccsc3n2CCC)c2c1CCC(C)(C)C2] | I-35 |
| 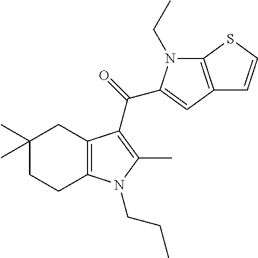 [SMILES: CCCn1c(C)c(C(=O)c2cc3ccsc3n2CC)c2c1CCC(C)(C)C2] | I-65 |
| 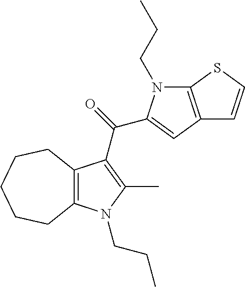 [SMILES: CCCn1c(C)c(C(=O)c2cc3ccsc3n2CCC)c2c1CCCCC2] | I-80 |
| 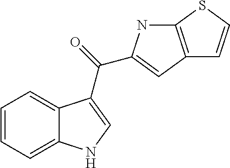 [SMILES: O=C(c1cc2ccsc2[nH]1)c1c[nH]c2ccccc12] | I-29 |
| 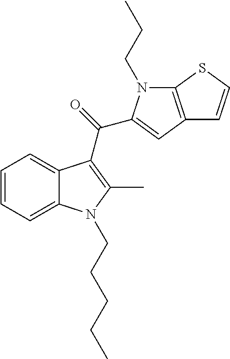 [SMILES: CCCCCn1c(C)c(C(=O)c2cc3ccsc3n2CCC)c2ccccc21] | I-60 |
| 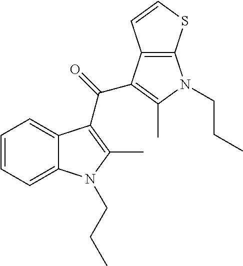 [SMILES: CCCn1c(C)c(C(=O)c2c(C)n(CCC)c3sccc23)c2ccccc21] | I-7 |
| 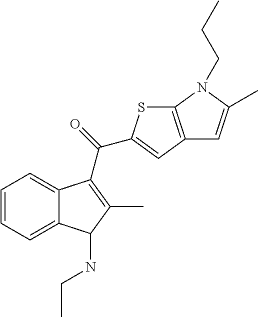 [SMILES: CCCn1c(C)cc2cc(C(=O)C3=C(C)C(NCC)c4ccccc43)sc21] | I-63 |
| 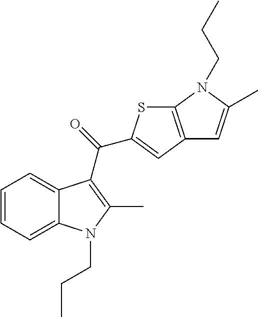 [SMILES: CCCn1c(C)c(C(=O)c2cc3cc(C)n(CCC)c3s2)c2ccccc21] | I-56 |
| 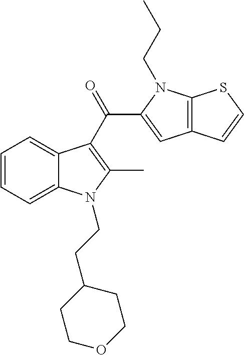 [SMILES: CCCn1c(C(=O)c2c(C)n(CCC3CCOCC3)c3ccccc23)cc2ccsc21] | I-57 |
| 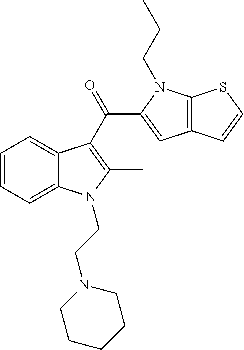 [SMILES: CCCn1c(C(=O)c2c(C)n(CCN3CCCCC3)c3ccccc23)cc2ccsc21] | I-59 |
| 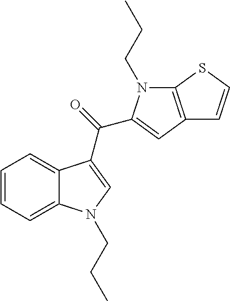 | I-62 |
| 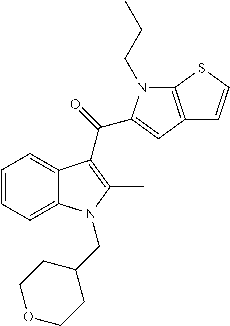 [SMILES: CCCn1c(C(=O)c2c(C)n(CC3CCOCC3)c3ccccc23)cc2ccsc21] | I-66 |
| 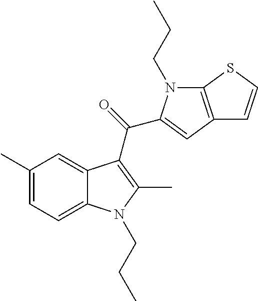 [SMILES: CCCn1c(C)c(C(=O)c2cc3ccsc3n2CCC)c2cc(C)ccc21] | I-69 |
| 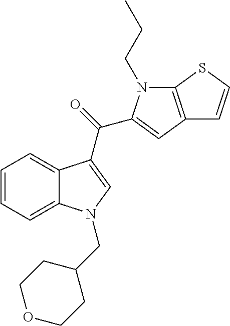 | I-41 |
| 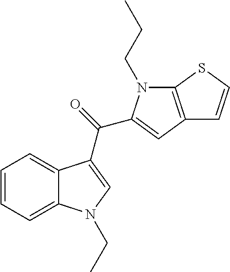 [SMILES: CCCn1c(C(=O)c2cn(CC)c3ccccc23)cc2ccsc21] | I-42 |
| 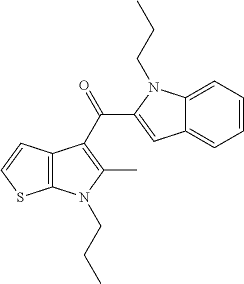 [SMILES: CCCn1c(C(=O)c2c(C)n(CCC)c3sccc23)cc2ccccc21] | I-14 |
| 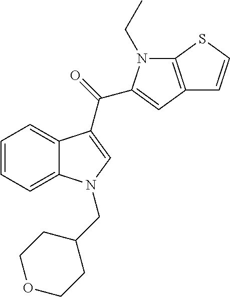 | I-16 |
| 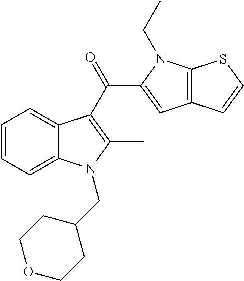 [SMILES: CCn1c(C(=O)c2c(C)n(CC3CCOCC3)c3ccccc23)cc2ccsc21] | I-17 |
| 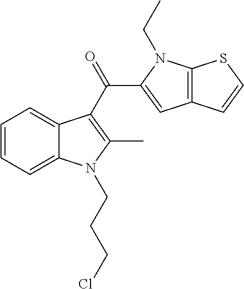 [SMILES: CCn1c(C(=O)c2c(C)n(CCCCl)c3ccccc23)cc2ccsc21] | I-18 |
| 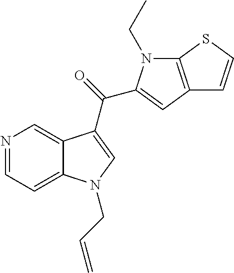 | I-21 |
| 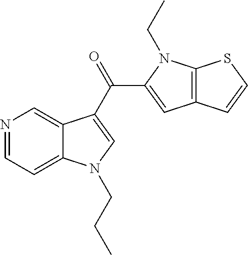 [SMILES: CCCn1cc(C(=O)c2cc3ccsc3n2CC)c2cnccc21] | I-22 |
| 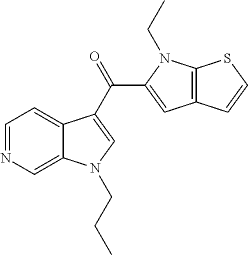 [SMILES: CCCn1cc(C(=O)c2cc3ccsc3n2CC)c2ccncc21] | I-23 |
| 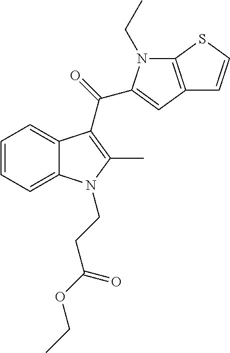 | I-26 |
| 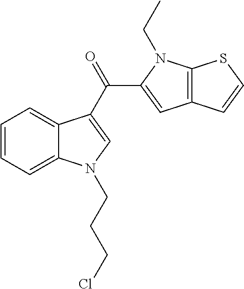 [SMILES: CCn1c(C(=O)c2cn(CCCCl)c3ccccc23)cc2ccsc21] | I-29 |
| 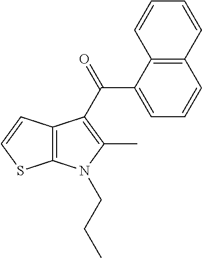 [SMILES: CCCn1c(C)c(C(=O)c2cccc3ccccc23)c2ccsc21] | I-2 |
| 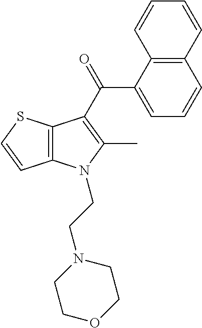 [SMILES: Cc1c(C(=O)c2cccc3ccccc23)c2sccc2n1CCN1CCOCC1] | I-1 |
| 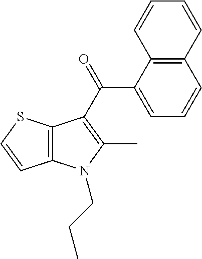 [SMILES: CCCn1c(C)c(C(=O)c2cccc3ccccc23)c2sccc21] | I-12 |
| 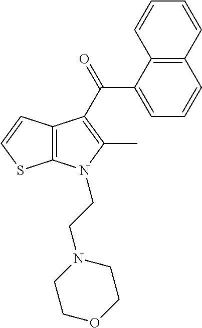 [SMILES: Cc1c(C(=O)c2cccc3ccccc23)c2ccsc2n1CCN1CCOCC1] | I-5 |
| 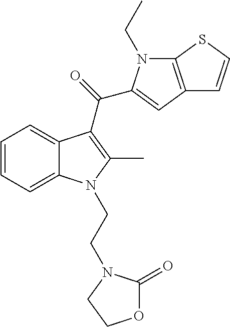 [SMILES: CCn1c(C(=O)c2c(C)n(CCN3CCOC3=O)c3ccccc23)cc2ccsc21] | I-25 |
| 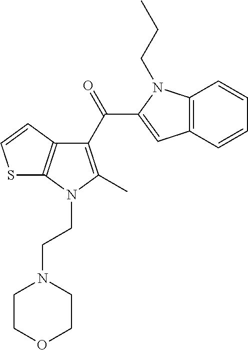 [SMILES: CCCn1c(C(=O)c2c(C)n(CCN3CCOCC3)c3sccc23)cc2ccccc21] | I-11 |
| 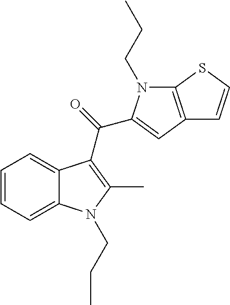 [SMILES: CCCn1c(C)c(C(=O)c2cc3ccsc3n2CCC)c2ccccc21] | I-78 |
| 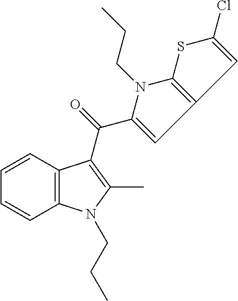 [SMILES: CCCn1c(C)c(C(=O)c2cc3cc(Cl)sc3n2CCC)c2ccccc21] | I-49 |
| 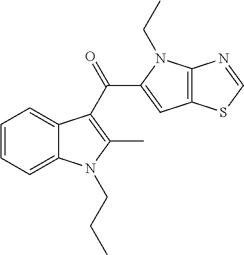 [SMILES: CCCn1c(C)c(C(=O)c2cc3scnc3n2CC)c2ccccc21] | I-31 |
| 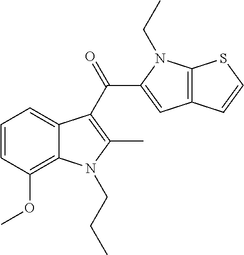 [SMILES: CCCn1c(C)c(C(=O)c2cc3ccsc3n2CC)c2cccc(OC)c21] | I-36 |
| 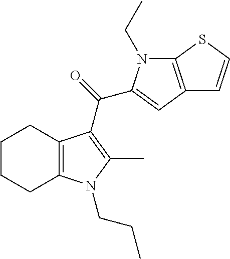 [SMILES: CCCn1c(C)c(C(=O)c2cc3ccsc3n2CC)c2c1CCCC2] | I-85 |
| 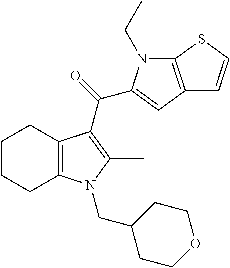 [SMILES: CCn1c(C(=O)c2c3c(n(CC4CCOCC4)c2C)CCCC3)cc2ccsc21] | I-86 |
| 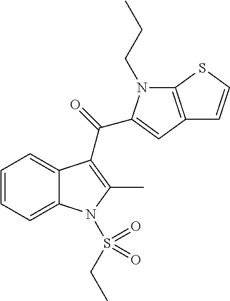 [SMILES: CCCn1c(C(=O)c2c(C)n(S(=O)(=O)CC)c3ccccc23)cc2ccsc21] | I-67 |
| 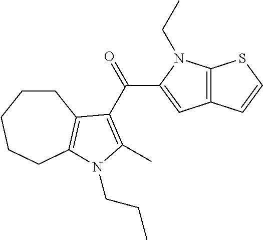 [SMILES: CCCn1c(C)c(C(=O)c2cc3ccsc3n2CC)c2c1CCCCC2] | I-87 |
| 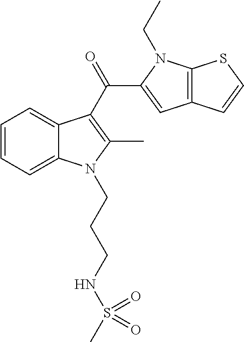 [SMILES: CCn1c(C(=O)c2c(C)n(CCCNS(C)(=O)=O)c3ccccc23)cc2ccsc21] | I-19 |
| 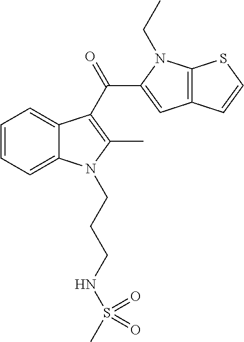 [SMILES: CCn1c(C(=O)c2c(C)n(CCCNS(C)(=O)=O)c3ccccc23)cc2ccsc21] | I-28 |
| 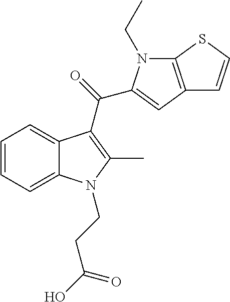 [SMILES: CCn1c(C(=O)c2c(C)n(CCC(=O)O)c3ccccc23)cc2ccsc21] | I-27 |
| 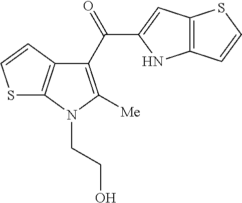 [SMILES: Cc1c(C(=O)c2cc3sccc3[nH]2)c2ccsc2n1CCO] | I-8 |
| 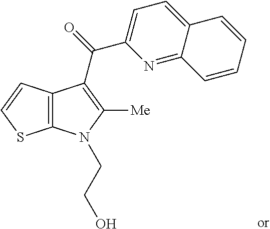 [SMILES: Cc1c(C(=O)c2ccc3ccccc3n2)c2ccsc2n1CCO ; "or"] | I-9 |
| 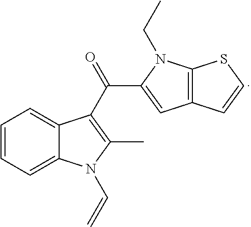 [SMILES: C=Cn1c(C)c(C(=O)c2cc3ccsc3n2CC)c2ccccc21] | I-24 |

24. A pharmaceutical composition comprising a compound according to claim 1, or a pharmaceutically acceptable salt thereof, and a pharmaceutically acceptable carrier, vehicle or adjuvant.

25. The pharmaceutical composition of claim 24, further comprising at least one additional therapeutic agent.

26. The pharmaceutical composition of claim 25, wherein the additional therapeutic agent is selected from the group consisting of pain-relieving agents, non-steroidal anti-inflammatory drugs (NSAIDs), cannabinoid-receptor agonists, opiate-receptor agonists, sodium-channel blockers. N-type calcium-channel blockers, local anesthetics, VR1 agonists and antagonists, agents used for migraine, anti-inflammatory and/or immunosuppressive agents, agents designed to treat tobacco abuse (e.g., nicotine-receptor partial agonists and nicotine replacement therapies), ADD/ADHD agents, agents to treat alcoholism, such as opioid antagonists, agents for reducing alcohol withdrawal symptoms such as benzodiazepines and beta-blockers, antihypertensive agents such as ACE inhibitors and Angiotensin II Receptor blockers, Renin inhibitors, vasodilators, agents used to treat glaucoma such as direct-acting Miotics (cholinergic, agonists), indirect-acting Miotics (cholinesterase inhibitors), Carbonic anhydrase inhibitors, selective adrenergic agonists, Osmotic diuretics, antidepressants such as SSRIs, tricyclic antidepressants, and dopaminergic antidepressants, cognitive improvement agents, acetylcholinesterase inhibitors, anti-emetic agents (e.g., 5HT3 antagonists), neuroprotective agents, neuroprotective agents currently under investigation, antipsychotic medications, agents used for multiple sclerosis, disease-modifying antirheumatic drugs (DMARDS), biological response modifiers (BRMs), COX-2-selective inhibitors, COX-1 inhibitors, immunosuppressives, PDE4 inhibitors, corticosteroids, histamine H1-receptor antagonists, histamine H2-receptor antagonists, proton-pump inhibitors, leukotriene antagonists, 5-lipoxygenase inhibitors, nicotinic acetylcholine receptor agonists, P2X3-receptor antagonists, NGF agonists and antagonists. NK1 and NK2 antagonists, NMDA antagonist, potassium-Channel modulators, GABA modulators, and serotonergic and noradrenergic modulators.

27-43. (canceled)

44. A method for the treatment or prevention of a disease or disorder comprising administering, alone or in combination therapy, to a patient in need thereof, a therapeutically or prophylactically acceptable dose of a pharmaceutical composition according to claim 1.

45. The method according to claim 44, wherein the disease or disorder is pain.

46. The method according to claim 45, wherein the pain is chronic pain, acute pain, perioperative pain (e.g., associated with surgery), postoperative pain, visceral pain, inflammatory pain, cancer pain, headache pain, neuropathic pain, dental pain (such as odontalgia), bone pain, joint pain (e.g., osteoarthritis or rheumatoid arthritis), myofascial pain (e.g., muscular injury, fibromyalgia), labor pain, pain associated with injuries, pain resulting from trauma, pain resulting from allergies, pain resulting from dermatitis, pain resulting from immunodeficiency, pain resulting from Hodgkin's disease, pain resulting from Myasthenia gravis, pain resulting from nephrotic syndrome, pain resulting from scleroderma, pain resulting from thyroiditis, central and peripheral pathway mediated pain, or pain associated with or the result of injury or age.

47. The method according to claim 44, wherein the disease or disorder is an autoimmune disorder.

48. The method according to claim 47, wherein the autoimmune disorder is selected from the group consisting of alopecia greata (also known as systemic sclerosis (SS)), amyloses, amyotrophic lateral sclerosis, ankylosing spondylarthritis, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, connective tissue diseases, crest syndrome, Crohn's disease, Dews disease, dermatomyositis-juvenile, discoid lupus, essential mixed cryoglobulinemia, fibromyalgia-fibromyositis, graft vs. host disease, transplantation rejection, Grave's disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, lupus erythematosus, Meniere's disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, reactional arthritis, Reiter's syndrome, rheumatic fever, rheumatoid arthritis, sarcoidosis, scleroderma (progressive systemic sclerosis (PSS), Sjogren's syndrome, stiff-man syndrome, systemic lupus erythematosus, Takayasu arteritis, temporal arteritis/giant cell arteritis, ulcerative colitis, undifferentiated spondylarthritis, uveitis, vitiligo, and Wegener's granulomatosis.

49. The method according to claim 44, wherein the disease or disorder is a disease-state or indication that is accompanied by an inflammatory process.

50. The method according to claim 49, wherein the disease-state or indication that is accompanied by an inflammatory process is chosen from the group consisting of
lung diseases such as asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral- or bacterial exacerbation of asthma, other non-allergic asthmas and “wheezy-infant syndrome”, pneumoconiosis, including aluminosis, anthracosis, asbestosis, chalicosis, ptilosis, siderosis, silicosis, tabacosis and byssinosis:
rheumatic diseases or autoimmune diseases or musculoskeletal diseases such as all forms of rheumatic diseases, especially rheumatoid arthritis, acute rheumatic fever, and polymyalgia rheumatica; reactive arthritis: rheumatic soft tissue diseases; inflammatory soft tissue diseases of other genesis; arthritic symptoms in degenerative joint diseases (arthroses); tendinitis, bursitis, osteoarthritis, traumatic arthritis, gout (metabolic arthritis); collagenoses of any genesis, e.g., systemic lupus erythematosus, scleroderma, polymyositis, dermatomyositis, Sjogren syndrome, Still disease, Felty syndrome; and osteoporosis and other bone resorption diseases;
allergic diseases including all forms of allergic reactions, e.g., allergic rhinitis, allergic conjunctivitis infectious parasitic, angioneurotic edema, hay fever, insect bites, allergic reactions to drugs, blood derivatives, contrast agents, etc., anaphylactic shock (anaphylaxis), urticaria, angioneurotic edema, delayed or immediate hypersensitivity, and contact dermatitis;
vascular diseases such as panarteritis nodosa, polyarteritis nodosa, periarteritis nodosa, arteritis temporalis, Wegner granulomatosis, giant cell arthritis, atherosclerosis, reperfusion injury and erythema nodosum;
dermatological diseases such as dermatitis, psoriasis, sunburn, burns, and eczema;
renal, urinary and pancreatic diseases such as nephrotic syndrome and all types of nephritis (such as glomerulonephritis); pancreatitis; bladder hyperrelexia following bladder inflammation;
hepatic diseases such as acute liver cell disintegration; acute hepatitis of various genesis (such as viral, toxic, drug-induced) and chronically aggressive and/or chronically intermittent hepatitis, liver fibrosis associated with liver injury or disease, including fibrosis caused or exacerbated by alcoholic liver cirrhosis, chronic viral hepatitis, non-alcoholic steatohepatitis and primary liver cancer;
gastrointestinal diseases such as inflammatory bowel diseases, irritable bowel syndrome, regional enteritis (Crohn's disease), colitis ulcerosa, gastritis, aphthous ulcer, celiac disease, regional ileitis, and gastroesophageal reflux disease;
neurodegenerative diseases such as in the treatment of neurodegeneration following stroke, cardiac arrest, pulmonary bypass, traumatic brain injury, spinal cord injury or the like;
eye diseases such as allergic keratitis, uveitis, or iritis, conjunctivitis, blepharitis, neuritis nervi optici, choroiditis, glaucoma and sympathetic ophthalmia;
diseases of the ear, nose, and throat (ENT) area such as tinnitus, allergic rhinitis or hay fever, otitis externa, caused by contact eczema, infection, etc., and otitis media;
neurological diseases such as brain edema, particularly tumor-related brain edema, multiple sclerosis, acute encephalomyelitis, meningitis, acute spinal cord injury, trauma, dementia, particularly degenerative dementia (including senile dementia, Alzheimer's disease, Parkinson's disease and Creutzfeldt-Jacob disease, Huntington's chorea, Pick's disease, motor neuron disease), vascular dementia (including multi-infarct dementia and dementia associated with intracranial space occupying lesions, infections and related, conditions such as HIV infection), Guillain-Barre syndrome, myasthenia gravis, stroke, and various forms of seizures (such as nodding spasms);
blood diseases such as acquired hemolytic anemia, aplastic anemia, and idiopathic thrombocytopenia;
tumor diseases such as acute lymphatic leukemia, Hodgkin's disease, malignant lymphoma, lymphogranulomatoses, lymphosarcoma, solid malignant tumors, and extensive metastases;
endocrine diseases such as endocrine opthalmopathy, endocrine orbitopathia, thyrotoxic crisis, Thyroiditis de Quervain, Hashimoto thyroiditis, Morbus Basedow, granulomatous thyroiditis, struma lymphomatosa, Graves' disease, type I diabetes (such as insulin-dependent diabetes); Organ and tissue transplantations and graft-versus-host diseases; and
severe states of shock such as septic shock, anaphylactic shock, and systemic inflammatory response syndrome (SIRS); and various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, granulocyte transfusion, sarcoidosis, gingivitis, pyrexia. Edema resulting from trauma associated with burns, sprains or fracture, cerebral edema and angioedema, and diabetes (such as diabetic vasculopathy, diabetic neuropathy, diabetic retinopathy, post capillary resistance and diabetic symptoms associated with insulitis (e.g., Hyperglycemia, diuresis, proteinuria and increased nitrite and kallikrein urinary excretion)).

51. The method according to claim 44, wherein the disease or disorder is a substance abuse related syndrome, disorder, disease or withdrawal symptom.

52. The method according to claim 51, wherein the substance abuse related syndrome, disorder, disease or withdrawal symptom is chosen from the group consisting of drug abuse and drug withdrawal, wherein the abused substances include alcohol, amphetamines, amphetamine like substances, caffeine, cannabis, cocaine, hallucinogens, inhalants, opioids, nicotine (and/or tobacco products), heroin abuse, barbiturates, phencyclidine (or phencyclidine-like compounds), sedative-hypnotics, benzodiazepines, or combinations of any of the foregoing; and the withdrawal symptoms include tobacco craving or nicotine dependency, addiction, or withdrawal.

53. The method according to claim 44, wherein the disease or disorder is a psychiatric disorder.

54. The method according to claim 53, wherein the psychiatric disorder is chosen from the group consisting of depression (including major depressive disorder, bipolar depression, unipolar depression, single or recurrent major depressive episodes (e.g., with or without psychotic features, catatonic features, and/or melancholic features), postpartum onset, seasonal affective disorder, dysthymic disorders (e.g., with early or late onset and with or without atypical features), neurotic depression and social phobia, depression accompanying dementia, anxiety, psychosis, social affective disorders, and or cognitive disorders), manic-depressive psychoses, bipolar disorders, extreme psychotic states (such as mania, schizophrenia, and excessive mood swings where behavioral stabilization is desired), attention disorders such as ADHD (attention deficit hyperactivity disorders), autism, anxiety states, generalized anxiety, agoraphobia, as well as those behavioral states characterized by social withdrawal.

55. The method according to claim 44, wherein the disease or disorder is a neurological or neurodegenerative disorder.

56. The method according to claim 55, wherein the neurological or neurodegenerative disorder is chosen from the group consisting of dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions: trauma; infections and related conditions (including HIV infection); dementia in Parkinson's disease, metabolism; toxins; anoxia and vitamin deficiency; and mild cognitive impairment associated with aging, particularly Age Associated Memory Impairment; amyotrophic lateral sclerosis (ALS), multiple sclerosis, epilepsy, ischemia, traumatic head or brain injury, brain inflammation, eye injury, stroke and neuroinflammation.

57. The method according to claim 44, wherein the disease or disorder is an ocular disorder.

58. The method according to claim 57, wherein the ocular disorder is chosen from the group consisting of glaucoma (such as normal tension glaucoma), glaucoma-associated intraocular pressure retinitis, retinopathies, uveitis, acute injury to the eye tissue (e.g., conjunctivitis), high intraocular pressure, family history of glaucoma, glaucoma in the contralateral eye and high myopia.

59. The method according to claim 44, wherein the patient is a human.

60. The method according to claim 44, wherein the patient is a companion animal, exotic animal or a farm animal such as a dog, cat, mouse, rat, hamster, gerbil, guinea pig, rabbit, horse, pig or cow.

61. A method of increasing CB receptor activity in a biological sample, comprising contacting said biological sample with a composition according to claim 44.
